(19) United States
(12) Patent Application Publication
(10) Pub. No.: US 2026/0297204 A1
(43) Pub. Date: Oct. 1, 2026

(54) HUMANIZED ANTIBODY TARGETING THE TUMOR ASSOCIATED ANTIGEN IL13RA2

(71) Applicant: Northwestern University, Evanston, IL (US)

(72) Inventor: Irina V. Balyasnikova, Chicago, IL (US)

(21) Appl. No.: 19/710,200

(22) Filed: Jun. 16, 2026

Related U.S. Application Data
(63) Continuation of application No. 17/995,966, filed on Apr. 12, 2021, now Pat. No. 12,703,750.
(60) Provisional application No. 63/008,681, filed on Apr. 11, 2020.

(51) Int. Cl. C07K 16/28; C07K 16/30

(52) U.S. Cl. CPC C07K 16/2866; C07K 16/2809; C07K 16/3053; C07K 2317/24; C07K 2317/31; C07K 2317/622; C07K 2317/92; C07K 2317/94

(57) ABSTRACT

The present invention provides humanized antibodies that bind to IL13Rα2, an interleukin-13 receptor that is overexpressed by the majority of glioblastoma tumors and not expressed at significant levels in normal brain tissue. Also provided are bispecific T cell engagers that bind to both IL13Rα2 and to the T cell co-receptor CD3 as well as methods for treating cancer, in which these humanized antibodies are used to target tumors.

### CROSS-REFERENCE TO RELATED APPLICATIONS

This application is a continuation of U.S. application Ser. No. 17/995,966, filed on Oct. 11, 2022, which is a U.S. national stage application filed under 35 U.S.C. § 371 of International Application No. PCT/US2021/070377, filed on Apr. 12, 2021, which claims priority to U.S. Provisional Application No. 63/008,681, filed on Apr. 11, 2020, the contents of which are incorporated by reference in their entireties.

### STATEMENT REGARDING FEDERALLY SPONSORED RESEARCH

This invention was made with government support under grant number CA221747 and grant number NS101150 awarded by the National Institutes of Health (NIH). The government has certain rights in this invention.

### REFERENCE TO AN ELECTRONIC SEQUENCE LISTING

The content of the electronic sequence listing (70258102813.xml; Size: 134,695 bytes; and Date of Creation: Jun. 16, 2026) is herein incorporated by reference in its entirety.

### BACKGROUND

Each year, tens of millions of people are diagnosed with cancer around the world, and more than half of them will eventually die from it. There is a search for new and effective cancer treatments. The selective killing of an individual cancer cells is desirable for cancer therapy where the goal of the treatment is for specifically targeting and killing tumor cells, while leaving healthy cells and tissues intact and undamaged. Antibodies that are able to deliver a drug or chemotoxic agent to cells are one avenue of research in cancer therapies. Further, methods of activating the cytotoxic immune response to tumor cells is being highly investigated for cancer treatments, including bispecific antibodies that can activate immune cells to destroy cancer cells. Bispecific antibodies designed to bind with one “arm” to a surface antigen on target cells, and with the second “arm” to an activating, invariant component of the T cell receptor (TCR) complex are under investigation as cancer therapeutics. Simultaneous binding to both of its targets forces a temporary interaction between target cell and T cell, causing activation of any cytotoxic T cell and subsequent lysis of the target cell, i.e. cancer cell.

Glioblastoma, also known as glioblastoma multiforme (GBM), is the most aggressive type of brain cancer. Despite recent advances in treatment, GBM remains largely incurable. The typical duration of survival following a GBM diagnosis is 12 to 15 months. Routine treatment of newly diagnosed GBM consists of surgical resection, chemotherapy, and radiation, which results in a median GBM patient survival of less than two years, with just 5% of patients surviving beyond five years. The blood-brain barrier (BBB) limits therapeutic access to the tumor. An immunosuppressive microenvironment and molecular heterogeneity of GBM present a unique set of challenges for developing effective therapies for this type of brain tumor.

The development of treatments for lessening the immunosuppressive effects of GBM represents an active area of preclinical and clinical neuro-oncology research. Many, if not all, approaches being tested involve increasing T cell cytotoxic antitumor activity. Large numbers of functional cytotoxic tumor-infiltrating lymphocytes (TILs) correlate with improved progression-free survival for GBM patients.

However, the immunosuppressive milieu of GBM impairs T cell cytolytic function, altering the effectiveness of T cell-based therapies for treating GBM. Numerous lymphocyte-directed treatments are being investigated, including the use of bispecific T cell engagers (BTE). BTEs can be produced and used without patient specific individualization and can, therefore, be considered “off-the-shelf” therapeutics. The use of BTEs targeting tumor-associated antigens (TAAs) has been approved by the Food and Drug Administration (FDA) in treating liquid malignancies, and BTE-associated treatments are currently being evaluated in multiple clinical studies for solid tumors (e.g., NCT03792841, NCT04117958, NCT03319940). BTEs consist of two single-chain variable fragments (scFvs) connected by a flexible linker. The specificity of BTE's tumor antigen-directed scFv is imperative to harness the full therapeutic potential of the recombinant molecule. BTE anti-cancer activity requires BTE binding with malignant and immune cells simultaneously; single-arm binding to a tumor antigen or CD3ε is therapeutically ineffective. However, the efficacy of the BTEs depends on the targeting ability and specificity to tumor cells.

Thus, there is an unmet need for effective therapeutic strategies for the treatment of GBM and targeting molecules that have specificity and affinity for the tumor cells to allow for specific killing of tumor cells.

### SUMMARY

The present disclosure provides engineered humanized and bispecific antibodies capable of binding IL13Rα2, compositions and methods of use for treating cancer, particularly glioblastoma.

In one aspect, the present disclosure provides a humanized antibody that binds to IL13Rα2 comprising: a variable light domain (V_{L}) comprising an amino acid sequence of SEQ ID NO:53 or an amino acid with at least 95% sequence similarity to SEQ ID NO:53; and a variable heavy domain (V_{H}) comprising an amino acid sequence of SEQ ID NO:54 or an amino acid with at least 95% sequence similarity to SEQ ID NO:54. The humanized antibody may have one or more mutation that improves the stability and the affinity of the antibody for IL13Rα2. In some aspects, the humanized antibody is a single chain antibody.

The present disclosure provides in another aspect, a humanized antibody that binds IL13Rα2 comprising: (a) a variable heavy domain (V_{H}) comprising SEQ ID NO:1, SEQ ID NO:2, SEQ ID NO:3, SEQ ID NO:4, SEQ ID NO:9, SEQ ID NO: 10, SEQ ID NO:11, SEQ ID NO:12, or an amino acid sequence having at least 95% sequence similarity to SEQ ID NO:1-4 or SEQ ID NO:9-12; and (b) a variable light domain (V_{L}) comprising SEQ ID NO:5, SEQ ID NO:6, SEQ ID NO:7, SEQ ID NO:8, SEQ ID NO:13, SEQ ID NO:14, SEQ ID NO: 15, SEQ ID NO:16, or an amino acid sequence having at least 95% sequence similarity to SEQ ID NO:5-8 or SEQ ID NO:13-16.

In another aspect, the present disclosure provides a humanized antibody comprises: (i) a V_{H} selected from the group consisting of SEQ ID NO:9, SEQ ID NO:10, SEQ ID NO:11, and SEQ ID NO:12, wherein the V_{H} has one or more mutations selected from M34L, M34A, M34I, M34V, D52E, P53A, D55E, and G56A; and (ii) a VL comprising the amino acid sequence selected from the group consisting of SEQ ID NO:13, SEQ ID NO:14, SEQ ID NO:15, and SEQ ID NO:16, wherein the V_{L} comprising one or more mutations selected from M37L, M37I, M37V, Q58R, Q58A, Q94E, Q94R, Q94A, W100F, and W100Y.

In a further aspect, the present disclosure provides a humanized antibody, wherein the antibody cannot isomerize and comprises: (i) a V_{H} selected from SEQ ID NO:9, SEQ ID NO:10, SEQ ID NO:11, or SEQ ID NO:12 wherein the V_{H} comprises G56A, and a V_{L} selected from SEQ ID NO:5, SEQ ID NO:6, SEQ ID NO:7, SEQ ID NO:8, SEQ ID NO:13, SEQ ID NO:14, SEQ ID NO:15, or SEQ ID NO:16; or (ii) a V_{H} selected from SEQ ID NO:9, SEQ ID NO:10, SEQ ID NO:11, or SEQ ID NO 12 wherein the V_{H} comprises D55E, and a V_{L} selected from SEQ ID NO:5, SEQ ID NO:6, SEQ ID NO:7, SEQ ID NO:8, SEQ ID NO:13, SEQ ID NO:14, SEQ ID NO:15, or SEQ ID NO 16.

In a further aspect, the disclosure provides a composition comprising the humanized antibody of any one of the preceding claims and a pharmaceutically acceptable carrier.

In another aspect, the disclosure provides a method of treating an IL13Rα2-expressing cancer in a subject, the method comprising: administering a therapeutically effective amount of the humanized antibody or the composition described herein to treat the cancer.

In yet another aspect, the disclosure provides a engineered bispecific antibody comprising a first single-chain variable fragment (scFv) that binds to CD3 and a second scFv that binds to IL13Rα2, wherein the first scFv comprises: (a) a variable light domain (V_{H}) comprising an amino acid sequence of SEQ ID NO:51 or an amino acid sequence with at least 95% sequence similarity to SEQ ID NO:51; (b) a first flexible linker; and (c) a variable heavy domain (V_{L}) comprising an amino acid sequence of SEQ ID NO:52 or an amino acid sequence with at least 95% sequence similarity to SEQ ID NO:52, and wherein the second scFv comprises: (d) a variable light domain (V_{L}) comprising an amino acid sequence of SEQ ID NO:53 or an amino acid with at least 95% sequence similarity to SEQ ID NO:53; (e) a second linker; and (f) a variable heavy domain (V_{H}) comprising an amino acid sequence of SEQ ID NO:54 or an amino acid with at least 95% sequence similarity to SEQ ID NO:54; and wherein the bispecific antibody comprises from 5′ to 3′: the V_{H} of the first scFv, the V_{L} of the first scFv, the V_{L} of the second scFv, and the V_{H} of the second scFv. In some aspects, the first single-chain variable fragment (scFv) that binds to CD3 and second scFv that binds to IL13Rα2 are linked via a third flexible linker.

In a further aspect, the disclosure provides transgenic neural stem cells (NSCs) that expresses the bispecific antibody described herein.

In yet another aspect, the disclosure provides a method of treating an IL13Rα2-expressing cancer in a subject, the method comprising: administering a therapeutically effective amount of the bispecific antibody described herein, the composition described herein, or the transgenic NSCs described herein to the subject to treat the cancer.

In a further aspect, the disclosure provides a method for inducing lysis of a target cell, particularly a tumor cell, comprising contacting a target cell with a bispecific antibody described herein in the presence of a T cell, particularly a cytotoxic T cell. In some embodiment, the method is in vivo.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 shows the results of running chimeric antibody on SDS-PAGE under reducing and non-reducing conditions. Lane M: Protein marker. Lane 1: Reducing condition (Purity: 99%). Lane 2: Non-reducing condition (Purity: 99%). Lane 3: Human IgG.

FIG. 2 shows sensor-grams of human IL13Rα2 binding to chimeric antibodies. Real-time responses are shown with colored curves. A fitting of Biacore experimental data to a 1:1 interaction model is shown in black. The antigen concentrations were 1.875, 3.75, 7.5, 15, 30, 60, 120 nM, respectively.

FIG. 3 shows sensor-grams of human IL13Rα2 binding to humanized antibodies. Real-time responses are shown with a red curve. A fitting of Biacore experimental data to a 1:1 interaction model is shown in black. The human IL13Rα2 concentration was 100 nM.

FIG. 4 shows the results of running three purified IgGs on SDS-PAGE under reducing and non-reducing conditions. Lane M: Protein marker. Lane 1: U3621EI120 VH2+VL3 under reducing conditions (Purity: 99%). Lane 2: U3621EI120 VH1+VL1 under reducing conditions (Purity: 99%). Lane 3: U3621EI120 VH1+VL4 under reducing conditions (Purity: 99%). Lane 4: U3621EI120 VH2+VL3 under non-reducing conditions (Purity: 98%). Lane 5: U3621EI120 VH1+VL1 under non-reducing conditions (Purity: 98%). Lane 6: U3621EI120 VH1+VL4 under non-reducing conditions (Purity: 97%). Lane 7: Human IgG.

FIG. 5 shows sensor-grams of human IL13Rα2 binding to chimeric and selected humanized antibodies. Real-time responses are shown with colored curves. A fitting of Biacore experimental data to a 1:1 interaction model is shown in black. The antigen concentrations were 1.875, 3.75, 7.5, 15, 30, 60, 120 nM for chimeric antibody, and 0.9375, 1.875, 3.75, 7.5, 15, 30, 60 nM for other antibodies, respectively.

FIG. 6 shows sensor-grams of human IL13Rα2 binding to selected antibodies.

FIG. 7 shows the results of a post-translational modification analysis performed on the sequence of the original mouse monoclonal antibody (mAb). The amino acid resides with potential liabilities (e.g., potential for deamidation, isomerization, etc.; see key) within the heavy chain variable region (V_{H}; top; SEQ ID NO:25) and the light chain variable region (V_{L}; bottom; SEQ ID NO:26) of this antibody are indicated with a colored bar below. The complementarity-determining regions (CDRs) are indicated with a gray bar above.

FIG. 8 shows the dynamic light scattering (DLS) graph of the chimeric antibody (Ab) at day 0 (DO).

FIG. 9 shows the DLS graph of the fusion protein comprising the single-chain variable fragments (scFvs) connected by flexible linker (VH1-VL1) at DO.

FIG. 10 shows the DLS graph of the D55E mutant scFv fusion protein (VH1D55E-VL1) at DO.

FIG. 11 shows the DLS graph of the G56A mutant scFv fusion protein (VH1G56A-VL1) at DO.

FIG. 12 shows the SEC-HPLC chromatograms at low pH 3.5 for Ab.

FIG. 13 shows the SEC-HPLC chromatograms at low pH 3.5 for VH1-VL1.

FIG. 14 shows the SEC-HPLC chromatograms at low pH 3.5 for VH1D55E-VL1.

FIG. 15 shows the SEC-HPLC chromatograms at low pH 3.5 for VH1D56A-VL1.

FIG. 16 shows the SEC-HPLC chromatogram at 40° C. for Ab.

FIG. 17 shows the SEC-HPLC chromatogram at 40° C. for VH1-VL1.

FIG. 18 shows the SEC-HPLC chromatogram at 40° C. for VH1D55E-VL1.

FIG. 19 shows the SEC-HPLC chromatogram at 40° C. for VH1D56A-VL1.

FIG. 20 shows the sensor-grams of antibodies to human IL13Rα2.

FIG. 21 shows binding of the chimeric murine antibody (Ab), humanized antibody (VH1-VL1) and variants of humanized antibody with mutations in the VH1 chain (D55E and G56A) to human recombinant IL13Rα2 in plate ELISA. N=3. Data are presented as mean SEM.

FIG. 22 shows a comparison of production of chimeric murine antibody (Ab), humanized antibody (VH1-VL1), and variants of humanized antibody with mutations in VH1 chain (D55E and G56A) in a single batch in the Expi293™ expression system.

FIG. 23 shows a comparison of binding of humanized antibody (VH1-VL1) and variants of humanized antibody with mutations in either the VH1 chain (M34A, D52E) or VL1 chain (M37A, Q58E, Q94E, W100F) to human recombinant IL13Rα2 in plate ELISA. N=4. Data is presented as mean±SEM.

FIG. 24 shows the production and binding of two mutant versions of the humanized antibody, which comprise either a single mutation in VL1 chain (W100F) or a double mutation (VH1 G56A-VL1 W100F). (A) Comparison of production of a single batch of humanized antibody (VH1-VL1), and the W100F and VH1 G56A-VL1 W100F variants thereof in the Expi293™ expression system. (B) Binding of the mutant antibodies to human IL13Rα2 in plate ELISA. N=4. Data presented as mean±SEM.

FIG. 25 depicts the generation of BTE^{on}. (A) Schematic of BTE^{on}, a bispecific fusion protein that comprises two single-chain variable fragments (scFvs) connected by flexible linker. The first scFv is derived from the fully human anti-CD3 antibody 28F1, and the second scFv is derived from the humanized anti-IL13Rα2 (clone 47) antibody described herein. (B) Image of the 293T/17 cells used to express BTE^{on}. (C) Protein gel showing purified BTE^{on}. (D) Binding of the humanized BTE to human IL13Rα2 in plate ELISA. Binding is compared to that of the murine version of the BTE, which comprises a first scFv derived from the anti-CD3 antibody Okt3 and a second scFv is derived from the murine anti-IL13Rα2 antibody.

FIG. 26 shows the results of a Chromium-51 (⁵¹Cr) release assay for BTE^{on} and BTE^{on}. Panel A shows the results generated using GBM6 cells, while panel B shows the results generated using GBM12 cells.

FIG. 27 demonstrates that BTE^{on} activates donor’ CD8+ T cells in co-culture with the IL13Rα2-expressing GBM6 patient-derived xenograft line, but not with IL13Rα2-negative GBM39 patient-derived xenograft line. Activation of T cells with CD3/CD28/CD2 beads (“Activated T cells) served as a positive control. N=4. Data presented as mean±SD

FIG. 28 are representative amino acid sequences and a polynucleic acid sequence of the bispecific T cell engagers contemplated in some embodiments of the present invention.

FIG. 29 is a representative graft depicting the affinity for L13Rα2 of a single and double mutant BTE (VH1G56-VL1 and VH1G56A-VL1W100F).

### DETAILED DESCRIPTION

Proteins that are expressed by tumor cells but not by normal cells are attractive molecular targets for the delivery of cytotoxic molecules to treat cancer. Antibodies are one promising means to target these tumor-specific proteins. Bispecific T cell engagers are also a further mean to target tumor cells and direct a cytotoxic immune response. In previous work, the inventor generated a mouse monoclonal antibody (mAb) against IL-13 receptor α2 (IL13Rα2), and demonstrated that the variable regions of the heavy chain (SEQ ID NO:25) and light chain (SEQ ID NO:26) of this antibody could be fused to functional moieties in a variety of configurations for therapeutic purposes (see U.S. Pat. No. 10,308,719, which is incorporated by reference in its entirety). IL13Rα2 is a monomeric high-affinity interleukin-13 (IL-13) receptor. Importantly, L13Rα2 is overexpressed by the majority of glioblastoma (GBM) tumors as well as several other tumor types, but is not expressed at significant levels on normal brain tissue.

In the present application, the inventor disclosed humanized antibody variants derived from the IL13Rα2-binding mouse monoclonal antibody. The inventor inserted the complementarity-determining regions (CDRs) of the mouse antibody into four different heavy chain variable region (V_{H}) human scaffolds and four different light chain variable region (V_{L}) human scaffolds, forming four humanized V_{H} regions (amino acid sequences: SEQ ID NO:1-4; DNA sequences: SEQ ID NO:17-20) and four humanized V_{L} regions (amino acid sequences: SEQ ID NO:5-8; DNA sequences: SEQ ID NO:21-24). The inventor expressed pairs of these humanized V_{H} and V_{L} regions to form 16 different humanized single-chain variable fragment (scFv) antibodies, each comprising one V_{H} and one V_{L} region (see Example 1). Surprisingly, several of the humanized scFv antibodies displayed improved binding affinity for IL13Rα2 as compared to the original murine scFv antibody. Further, the humanized antibodies were further altered in their amino acid sequence to provide additional benefits for use of the antibodies for human treatment, including longer-term stability and increased affinity for their receptor, increasing their desirability and potency as a therapeutic. For example, removal of certain post-translational modifications may stabilize the antibody product, as described more herein. The two main changes described herein is the altering of the amino acids (DG) that form an isomerization site in the antibodies and the removal of an oxidation site (e.g., tryptophan within the CDRs (i.e., W100F of the variable light chain). Isomerization can decrease binding affinities of the antibodies and reduce the stability of the polypeptides, thus the ability to decrease the isomerization of the humanized antibodies and bispecific antibodies described herein results in improved binding and stability. Tryptophan (Trp) has unique hydrophobic and structural properties, especially when positioned within the CDR. However, oxidation of Trp residues within the CDR can deleteriously impact antigen binding especially if it alters the CDR confirmation (see, e.g., Hageman et al. Impact of Tryptophan oxidation in complementarity-determining regions of two monoclonal antibodies on structure-function characterized by hydrogen-deuterium exchange mass spectrometry and surface plasmon resonance, Pharm Res (2019) 36:24). The removal of the oxidation site as described herein may allow for increased stability of the humanized antibody products produced while retaining the affinity to L13Rα2 (see Table 16). Humanized antibodies offer several advantages for clinical use: they are less immunogenic that their mouse counterparts, they show improved serum half-life, and they produce better therapeutic outcomes. Thus, with their improved ability to target IL13Rα2, the humanized antibodies of the present invention are promising therapeutic tools for the treatment of GBM and other cancers.

The potency of therapeutic antibodies can be diminished by the isomerization of specific aspartic acid residues. When isomerization occurs in a complementarity-determining region (CDR), it can decrease the binding affinities of these antibodies to their ligands. Further, isomerization can reduce the stability of these proteins, which becomes problematic during prolonged storage. When the present inventor analyzed the post-translational modifications of their humanized antibodies, they identified an aspartic acid residue (D55) and glycine reside (G56) within a CDR2 of the heavy chain that is susceptible to isomerization due to the presence of a glycine residue at its C-terminal end. Accordingly, the inventor generated point mutations at both aspartic acid residue (D55E; see, e.g., SEQ ID NO:27, SEQ ID NO:54) and the downstream glycine residue (G56A; see, e.g., SEQ ID NO:28, SEQ ID NO:54) and tested the binding affinity of the resultant antibodies (see Example 2). This analysis revealed that while the D55E mutation produced an antibody with decreased binding affinity, the G56A mutation did not significantly affect affinity. However, antibodies comprising the D55E mutation showed less reduction in binding after prolonged storage at warmer temperatures, suggesting that it may offer an improved in vivo half-life at physiological body temperature. Thus, removal of this isomerization site may be a critical step in adapting these antibodies for use in therapeutic applications, as demonstrated in the examples. The stability of these antibodies was tested demonstrating that they have higher melting temperatures and better stability for storage and use conditions than the chimeric antibodies.

The inventor inspected the sequences of the humanized antibodies for other sites that could affect binding activity, such as N-glycosylation sites, post-translational modifications, and unpaired cysteine residues (see Example 3). These sites, which are indicated with a numbered X in modified versions of the V_{H} (SEQ ID NO:9-12, 54) and V_{L} (SEQ ID NO:13-16, 53) sequences disclosed herein, which can be used to generate additional variants of the humanized antibodies with specific point mutations. These mutations may provide improved stability for the antibodies, especially for in vivo administration and use.

### Antibodies:

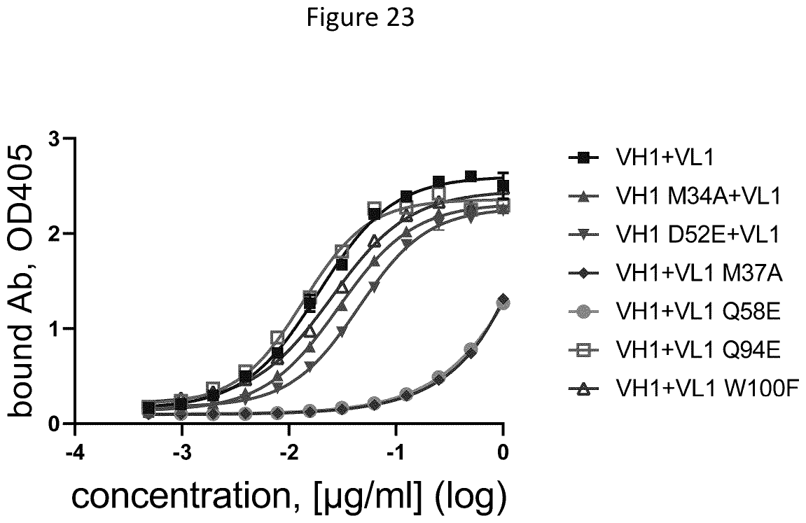

The present invention provides humanized antibodies that bind IL13Rα2. The inventor has engineered a number of different variants of humanized antibodies or fragments thereof that have better infinities for the receptor (IL13Rα2), increased stability and reduced isomerization, which provide improved properties that are desirable for human therapeutics. The inventor specifically found that mutations to disrupt a potential isomerization hot spot (e.g., DG in position 55 and 56 of SEQ ID NO:54) and a mutation at W100 (e.g., W100F of SEQ ID NO:53) as a potential. These mutations provided additional benefits to the antibodies and bispecific antibodies (T cell engagers) of the present technology by increasing binding, stability and purity of the product. The inventor surprisingly found that other mutations that alter other post-modification sites drastically reduced the binding and production of the antibodies (e.g., M37A and Q58E, as demonstrated in FIG. 23). Thus, the inventor has engineered mutant antibodies and fragments thereof that provide enhanced properties for use as therapeutics.

In one embodiment, the disclosure provides engineered humanized antibodies that binds to IL13Rα2 comprising: a variable light domain (V_{L}) comprising an amino acid sequence of SEQ ID NO:53 or an amino acid with at least 95% sequence similarity to SEQ ID NO:53; and a variable heavy domain (V_{H}) comprising an amino acid sequence of SEQ ID NO:54 or an amino acid with at least 95% sequence similarity to SEQ ID NO:54. In some embodiments, (a) X₁ in V_{L} is F; (b) X₂ in V_{H} is E, (c) X₃ in V_{H} is A; or (d) combinations of (a), (b) and (c). In a preferred embodiment, X₁ in V_{L} is F; and X₃ in V_{H} is A. Suitably, in a preferred embodiment, the humanized antibody is a single chain antibody and further comprising a flexible linker between the V_{H} and V_{L} domain.

Suitable flexible linkers can be determined by one skilled in the art and include, for example, an amino acid sequence from 4-25 amino acids in length, and preferably comprising, for example, glycine and serine.

In another aspect, the antibodies comprise (a) a variable heavy domain comprising SEQ ID NO:1, SEQ ID NO:2, SEQ ID NO:3, SEQ ID NO:4, SEQ ID NO:9, SEQ ID NO:10, SEQ ID NO:11, SEQ ID NO:12, or an amino acid sequence having at least 95% sequence similarity to SEQ ID NO:1-4 or SEQ ID NO:9-12; and (b) a variable light domain comprising SEQ ID NO:5, SEQ ID NO:6, SEQ ID NO:7, SEQ ID NO:8, SEQ ID NO:13, SEQ ID NO:14, SEQ ID NO:15, SEQ ID NO:16, or an amino acid sequence having at least 95% sequence similarity to SEQ ID NO:5-8 or SEQ ID NO:13-16.

The term “antibody” is used herein to refer to immunoglobulin molecules or other molecules that comprise an antigen-binding domain from an immunoglobulin molecule. Suitable antibody molecules include, without limitation, whole antibodies (e.g., IgG, IgA, IgE, IgM, or IgD), monoclonal antibodies, humanized antibodies, and antibody fragments, including single chain variable fragments (ScFv), single domain antibodies, antigen-binding fragments (e.g., complementarity determining region (CDR) domains), and genetically engineered antibodies. Thus, the humanized antibodies of the present invention may be configured as any form of antibody, antibody fragment, or antibody-derived fragment, as long as they retain the ability to bind IL13Rα2. Antibody binding may be assessed using any appropriate assay including, for example, surface plasmon resonance (SPR), radioimmunoassay, flow cytometry, enzyme-linked immunosorbent assays (ELISA), fluorescence immunoassay (FIA), thermal shift assay, LC-MS detection, and kinetic exclusion assays (KinExA).

As stated above, the term “antibody” includes “antibody fragments” or “antibody-derived fragments” that comprise an antigen-binding domain. As used herein, the term “antibody fragment” is intended to include any fragment that displays antigen (i.e., IL13Rα2) binding function, for example, Fab, Fab′, F(ab′)2, scFv, Fv, dsFv, ds-scFv, Fd, dAbs, TandAbs dimers, mini bodies, monobodies, diabodies, and multimers thereof and bispecific antibody fragments. As used herein, the term “fragment” refers to fragments of biological relevance (i.e., functional fragments). For example, the fragments may contribute to or enable antigen binding, form part of or all of an antigen binding site, or contribute to the prevention of the antigen interacting with its natural ligand.

The antibodies disclosed herein comprise at least a heavy chain variable region (V_{H}), which generally comprises the antigen-binding site, and a light chain variable region (V_{L}). However, the antibodies may further comprise additional antibody regions. For example, the antibodies can be made such that they also comprise all or a portion of a heavy chain constant region, such as an IgG1, IgG2, IgG3, IgG4, IgAQ1, IgA2, IgE, IgM or IgD constant region. Furthermore, the antibody or antibody fragment can comprise all or a portion of a kappa light chain constant region or a lambda light chain constant region. The V_{H} and V_{L} sequences disclosed herein can be genetically engineered into antibodies and antibody fragments using conventional techniques, including recombinant or chemical synthesis techniques, which are well known and described in the art.

Importantly, the antibodies of the present invention are humanized antibodies. The term “humanized antibody” refers to antibodies in which the human antibody framework has been modified to comprise fragments of antibodies taken from a different species (i.e., mouse) that provide antigen specificity. This term includes chimeric antibodies containing minimal sequence derived from non-human immunoglobulin. For example, the hypervariable region residues of a human antibody may be replaced by hypervariable region residues from a non-human species having the desired specificity, affinity, and capacity. For example, the present inventor created humanized antibodies by inserting the complementarity-determining regions (CDRs) of a previously disclosed mouse antibody into several different heavy chain variable region (V_{H}) and light chain variable region (V_{L}) human scaffolds. In some instances, framework region (FR) residues of the human immunoglobulin are replaced by corresponding non-human residues. In some instances, humanized antibodies may comprise residues that are not found in the recipient antibody or in the donor antibody and are included to refine antibody performance. In general, the humanized antibody will comprise substantially all of at least one, and typically two or three variable domains, in which all or substantially all of the hypervariable loops correspond to those of a non-human immunoglobulin and all or substantially all of the FR regions are those of a human immunoglobulin sequence. In some embodiments, the humanized antibody will also optionally comprise at least a portion of an immunoglobulin constant region (Fc), typically that of a human immunoglobulin. Appropriate sequences for such constant regions are well known and documented in the art. For further details, see Jones et al., Nature 321:522-525 (1986); Reichmann et al., Nature 332:323-329 (1988); and Presta, Curr. Op. Struct. Biol. 2:593-596 (1992).

In the Examples, the humanized antibodies were expressed as single-chain variable fragment (scFv) antibodies. As used herein, the term “single-chain variable fragment” or “scFv” refers to a fusion protein of the variable regions of the heavy (V_{H}) and light chains (V_{L}) of immunoglobulins, connected with a short linker peptide of about ten to about 25 amino acids. The linker may be rich in glycine for flexibility, as well as serine or threonine for solubility. The linker can either connect the N-terminus of the V_{H} with the C-terminus of the V_{L}, or vice versa. ScFvs may be produced in a cell culture of microbes such as Escherichia coli or Saccharomyces cerevisiae. ScFvs can also be produced in tissue culture, for example, in a mammalian or human cell line. ScFvs have many uses, e.g., therapeutic, flow cytometry, immunohistochemistry, and as antigen-binding domains of artificial T cell receptors. Thus, in some embodiments, the humanized antibody is a single chain antibody (ScFv), and in some embodiments, the heavy variable region (V_{H}) and the light variable region (V_{L}) are linked by a flexible linker. In some embodiments, the sequences of the variable regions (i.e., V_{H} and V_{L}) may further comprise a signal sequence, e.g., the signal sequence from mouse Ig heavy chain V region 102 (e.g., amino acid sequence: SEQ ID NO:29; nucleotide sequence: SEQ ID NO:46). Other suitable signal sequences are contemplated for use and known in the art, and can be located 5′ to the variable heavy domain. Signal sequences allow for the secretion of the humanized antibody, and are cleaved during maturation of the antibody when secreted from the cells into the extracellular space.

In certain embodiments, the humanized antibodies comprise the scFvs tested in Example 1 or described in Examples 4-6. In these embodiments, the antibodies comprise: (i) SEQ ID NO:1 and SEQ ID NO:5; (ii) SEQ ID NO:1 and SEQ ID NO:6; (iii) SEQ ID NO:1 and SEQ ID NO: 7; (iv) SEQ ID NO:1 and SEQ ID NO:8; (v) SEQ ID NO:2 and SEQ ID NO:5; (vi) SEQ ID NO:2 and SEQ ID NO:6; (vii) SEQ ID NO:2 and SEQ ID NO:7; (viii) SEQ ID NO:2 and SEQ ID NO: 8; (ixv) SEQ ID NO:3 and SEQ ID NO:5; (x) SEQ ID NO:3 and SEQ ID NO:6; (xi) SEQ ID NO: 3 and SEQ ID NO:7; (xii) SEQ ID NO:3 and SEQ ID NO:8; (xiii) SEQ ID NO:4 and SEQ ID NO: 5; (xiv) SEQ ID NO:4 and SEQ ID NO:6; (xv) SEQ ID NO:4 and SEQ ID NO:7; and (xvi) SEQ ID NO:4 and SEQ ID NO:8. In some embodiments, the scFvs are antibodies that comprise amino acid sequences with at least 95% sequence identity to the SEQ IDs listed, and the antibodies retain their ability to bind to [L13Rα2.

The humanized antibodies of the present invention may be from any appropriate source. The antibodies can be produced in vitro or in vivo, and can be wholly or partially synthetically produced. For example, the antibodies may be from a recombinant source and/or produced in transgenic cells, animals or transgenic plants.

As discussed above, the binding affinity of an antibody can be compromised when sites within functional regions (e.g., CDRs) undergo chemical changes such as isomerization or post-translational modification. In Example 3, the inventor identified specific residues that are susceptible to such chemical changes. These residues, which are indicated with a numbered X in generic versions of the V_{H} (SEQ ID NO:9-12) and V_{L} (SEQ ID NO:13-16) sequences disclosed herein, include methionine residues that are susceptible to oxidation (M34 in the V_{H} sequences, M37 in the V_{L} sequences), an aspartic acid (D52 in V_{H}) and downstream proline residue (P53 in V_{H}) that form a potential hydrolysis site, an aspartic acid (D55 in V_{H}) and downstream glycine residue (G56 in V_{H}) that form a potential isomerization site, glutamine residues that are susceptible to deamination (Q58 and Q94 in V_{L}), and a tryptophan residue (W100 in V_{L}) that is susceptible to oxidation.

Thus, in some embodiments, the humanized antibodies comprise point mutations that have been designed to prevent chemical changes at these susceptible residues. Specifically, in some embodiments, the humanized antibodies comprise: (i) a V_{H} selected from the group consisting of SEQ ID NO:9, SEQ ID NO:10, SEQ ID NO:11, or SEQ ID NO:12, wherein the V_{H} has one or more mutations selected from M34L, M34A, M34I, M34V, D52E, P53A, D55E, and G56A; or (ii) a V_{L} comprising the amino acid sequence selected from the group consisting of SEQ ID NO: 13, SEQ ID NO:14, SEQ ID NO:15, and SEQ ID NO:16, wherein the V_{L}, comprising one or more mutations selected from M37L, M37I, M37V, Q58R, Q58A, Q94E, Q94R, Q94A, W100F, or W100Y. In some embodiments, the humanized antibody comprises (i) a V_{H} selected from the group consisting of SEQ ID NO:9, SEQ ID NO:10, SEQ ID NO:11, or SEQ ID NO:12, wherein the V_{H} has one or more mutations selected from M34L, M34A, M34I, M34V, D52E, P53A, D55E, and G56A; or (ii) a V_{L} comprising the amino acid sequence selected from the group consisting of SEQ ID NO:13, SEQ ID NO:14, SEQ ID NO:15, and SEQ ID NO:16, wherein the V_{L} comprising two or more mutations selected from M37L, M37I, M37V, Q58R, Q58A, Q94E, Q94R, Q94A, W100F, or W100Y. In some embodiments, the humanized antibody comprises (i) a V_{H} selected from the group consisting of SEQ ID NO:9, SEQ ID NO:10, SEQ ID NO: 1l, or SEQ ID NO:12, wherein the V_{H} has two or more mutations selected from M34L, M34A, M34I, M34V, D52E, P53A, D55E, and G56A; or (ii) a V_{L} comprising the amino acid sequence selected from the group consisting of SEQ ID NO:13, SEQ ID NO:14, SEQ ID NO:15, and SEQ ID NO:16, wherein the V_{L} comprising two or more mutations selected from M37L, M37I, M37V, Q58R, Q58A, Q94E, Q94R, Q94A, W100F, or W100Y. In some embodiments, the humanized antibody comprises (i) a V_{H} selected from the group consisting of SEQ ID NO:9, SEQ ID NO:10, SEQ ID NO:11, or SEQ ID NO:12, wherein the V_{H} has two or more mutations selected from M34L, M34A, M34I, M34V, D52E, P53A, D55E, and G56A; or (ii) a V_{L} comprising the amino acid sequence selected from the group consisting of SEQ ID NO:13, SEQ ID NO:14, SEQ ID NO:15, and SEQ ID NO:16, wherein the V_{L} comprising one or more mutations selected from M37L, M37I, M37V, Q58R, Q58A, Q94E, Q94R, Q94A, W100F, or W100Y. The present invention contemplates where each of the V_{H} and V_{L} may contain one or more, two or more, three or more, four or more, five or more, six or more of the mutations described and any combinations thereof (e.g., one mutation in V_{H} and one mutation in V_{L}, two mutations in V_{H} and only one mutation in V_{L}, etc.) and that the combinations are not limited to the exemplary embodiments described herein.

In certain embodiments, the humanized antibodies comprise point mutations that disrupt the potential isomerization site formed by an aspartic acid (D55) and downstream glycine residue (G56) in the V_{H} sequences disclosed herein, which were tested in Example 2. In these embodiments, the humanized antibodies cannot isomerize and comprises (i) a V_{H} selected from SEQ ID NO:9, SEQ ID NO:10, SEQ ID NO:11, or SEQ ID NO:12 wherein the V_{H} comprises G56A, and a V_{L} selected from SEQ ID NO:5, SEQ ID NO:6, SEQ ID NO:7, SEQ ID NO:8, SEQ ID NO:13, SEQ ID NO:14, SEQ ID NO 15, or SEQ ID NO:16; (ii) a V_{H} selected from SEQ ID NO: 9, SEQ ID NO:10, SEQ ID NO: 1l, or SEQ ID NO:12 wherein the V_{H} comprises D55E, and a V_{L} selected from SEQ ID NO:5, SEQ ID NO:6, SEQ ID NO:7, SEQ ID NO:8, SEQ ID NO:13, SEQ ID NO:14, SEQ ID NO: 15, or SEQ ID NO:16, or (iii) a V_{H} selected from SEQ ID NO:9, SEQ ID NO:10, SEQ ID NO:11, or SEQ ID NO:12 wherein the V_{H} comprises D55E and G56A, and a V_{L} selected from SEQ ID NO:5, SEQ ID NO:6, SEQ ID NO:7, SEQ ID NO:8, SEQ ID NO:13, SEQ ID NO:14, SEQ ID NO:15, or SEQ ID NO: 16.

The inventor discovered that many of the humanized ScFv antibodies they tested have a greater affinity for IL13Rα2 than the original mouse ScFv antibody. Affinity, the strength with which a molecule binds to its ligand, refers to the strength of the sum total of non-covalent interactions between a single binding site of a molecule and its binding partner and is typically measured and reported as an equilibrium dissociation constant (K_{D}). K_{D} is the ratio of the antibody dissociation rate (k_{d}, i.e., how quickly it dissociates from its antigen) to the antibody association rate (kₐ, i.e., how quickly it binds to its antigen). Thus, K_{D} and affinity are inversely related, such that a lower K_{D} value indicates a higher affinity and a higher K_{D} value indicates a lower affinity. In the Examples, the K_{D} was determined for each antibody using a surface plasmon resonance (SPR) biosensor to measure the dissociation (k_{d}) and association (kₐ) rate constants. Accordingly, in some embodiments, the humanized antibody comprise an antibody with a K_{D} that is less than that that of the original mouse antibody (i.e., less than 5×10⁻⁹, preferably less than 1×10⁻¹⁰). In some embodiments, the humanized antibody or bispecific antibody has a binding constant (K_{D}) of 5×10⁻⁹ M or less for IL13Rα2. In some embodiments, the humanized antibody has a binding constant (K_{D}) of 2×10⁻¹⁰ M or less for IL13Rα2.

With their affinity for IL13Rα2, the humanized antibodies of the present invention are useful for targeting IL13Rα2-expressing cancer cells. This ability can be harnessed to deliver other molecules to cancer cells. Thus, in some embodiments, the humanized antibodies further comprise an agent. The term “agent,” as used herein, includes any useful moiety that allows for the purification, identification, detection, diagnosing, imaging, or therapeutic use of the antibodies of the present invention. The agent is selected according to the intended application (e.g., treatment of a particular cancer) and may be covalently or non-covalently connected to the antibody. In one embodiment, the agent conjugated to the antibody forming an antibody-conjugate. Methods of conjugating antibodies to compounds are described below. Additionally, the agent may be connected to the antibody by genetically fusing the agent via creation of a fusion protein as described below.

In some embodiments, the agent is a therapeutic agent. Exemplary therapeutic agents include, without limitation, pharmaceuticals, biologics, toxins, fragments of toxins, alkylating agents, enzymes, antibiotics, antimetabolites, antiproliferative agents, chemotherapeutic agents, hormones, neurotransmitters, DNA, RNA, siRNA, oligonucleotides, antisense RNA, aptamers, lectins, compounds that alter cell membrane permeability, photochemical compounds, small molecules, liposomes, micelles, gene therapy vectors, viral vectors, immunological therapeutic constructs, and other drugs. Drugs that treat cancer are particularly suitable for use in the present invention.

In other embodiments, the agent is a detection agent. Suitable detection agents include, without limitation, epitope tags, detectable markers, radioactive markers, and nanoparticles. Suitable epitope tags are known in the art and include, but are not limited to, 6-Histidine (His), hemagglutinin (HA), cMyc, GST, Flag tag, V5 tag, and NE-tag, among others. Epitope tags are commonly used as a purification tags (i.e., an agent that allows isolation of the antibody from other non-specific proteins). Suitable detectable markers include luminescent markers, fluorescent markers (e.g., fluorescein, fluorescein isothiocyanate, rhodamine, dichlorot[pi]azinylamine fluorescein, green fluorescent protein (GFP), red fluorescent protein (RFP), blue fluorescent dyes excited at wavelengths in the ultraviolet (UV) part of the spectrum (e.g., AMC-A (7-amino-4-methylcoumarin-3-acetic acid); Alexa Fluor 350), green fluorescent dyes excited by blue light (e.g., FITC, Cy2, Alexa Fluor 488), red fluorescent dyes excited by green light (e.g., rhodamines, Texas Red, Cy3, Alexa Fluor dyes 546, 564 and 594), or dyes excited with infrared light (e.g., Cy5), dansyl chloride, and phycoerythrin), or enzymatic markers (e.g., horseradish peroxidase, alkaline phosphatase, beta-galactosidase, glucose-6-phosphatase, and acetylcholinesterase). Suitable radioactive markers include, but are not limited to, ¹²⁵I, ¹³¹I, ³⁵S or ³H. Suitable nanoparticles, including metal nanoparticles and other metal chelates, are known in the art and include, but are not limited to, gold nanoparticles (ACSNano, Vol. 5, No. 6, 4319-4328, 2011), quantum dots (Nanomedicine, 8 (2012) 516-525), magnetic nanoparticles (Fe₃O₄), silver nanoparticles, nanoshells, and nanocages.

Methods of conjugating, linking and coupling antibodies to compounds are well known in the art, see Nat Biotechnol. (2005) 23(9):1137-46; Cancer Immunol Immunother. (2003) 52(5):328-37; and Adv Drug Deliv Rev. (2003) 55(2):199-215. For example, one may wish to link the antibodies of the present invention to an agent via primary amines (see Pharmaceutical Research (2007) 24(9): p. 1759-1771). For example, lysine residues of either antibody or agent may be functionalized using Traut's reagent (2-iminothiolane.HCL) yielding a thiol. The thiol group, now attached to the lysine residue, is reacted with a maleimide-functionalized drug or vector resulting in a stable thio-ether bond. One may optionally use a chemical spacer such as poly-ethylene glycol to reduce steric hindrance. Alternatively, one may wish to link the antibody to the agent non-covalently. For example, one could use biotin/streptavidin interaction (see Pharmaceutical Research (2007) 24(9): p. 1759-1771, incorporated by reference in its entirety). Lysine residues of either the antibody or agent may be biotinylated using one of a number of commercial methods (e.g., N-hydroxysuccinimide biotin analogs). Then, either the antibody or the agent (whichever one was not modified in the previous step) would be conjugated to streptavidin or one of its variants (e.g., neutravidin). The monobiotinylated reagent and the streptavidin-conjugated counterpart would be combined and the near-covalent binding affinity would keep the reagents together. Conjugation may optionally be accomplished with a cleavable or non-cleavable linker. Many chemical cross-linking methods are also known in the art. Cross-linking reagents may be homobifunctional (i.e., having two functional groups that undergo the same reaction) or heterobifunctional (i.e., having two different functional groups). Numerous cross-linking reagents are commercially available, and detailed instructions for their use are readily available from the commercial suppliers. For a general reference on polypeptide cross-linking and conjugate preparation, see Wong, Chemistry of protein conjugation and cross-linking, CRC Press (1991).

In some embodiments, the agent is a polypeptide that is translated concurrently with the antibody polypeptide sequence as a fusion protein. In such embodiments, the agent is “genetically fused” to the antibody. For example, one may wish to express the antibody as a fusion protein with a therapeutic peptide. Standard molecular biology techniques (e.g., restriction enzyme based subcloning or homology based subcloning) can be used to insert the DNA sequence encoding the agent in frame with the targeting vector. Optionally, a protein linker may be added to avoid steric hindrance. The fusion protein is then produced as one peptide in a cell (e.g., yeast, bacteria, insect, or mammalian cell) and purified before use. Note that the agent does not need to be a whole protein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al, Kuby Immunology, 6^{th} ed., W.H. Freeman and Co., page 91 (2007). A single VH or VL domain may be sufficient to confer antigen-binding specificity. The term “hypervariable region” or “HVR” are also referred to as “complementarity determining regions” (CDRs), and these terms are used herein interchangeably in reference to portions of the variable region that form the antigen binding regions. These regions of an antibody variable domain are hypervariable in sequence and/or form structurally defined loops (“hypervariable loops”). Generally, native four-chain antibodies comprise six CDRs; three in the VH (H1, H2, H3), and three in the VL (L1, L2, L3). With the exception of CDR1 in VH, CDRs generally comprise the amino acid residues that form the hypervariable loops. This particular region has been described by Kabat et al, U.S. Dept. of Health and Human Services, Sequences of Proteins of Immunological Interest (1983) and by Chothia et al, J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers that encompass a particular CDR will vary depending on the sequence and size of the CDR. Those skilled in the art can routinely determine which residues comprise a particular CDR given the variable region amino acid sequence of the antibody.

### Bispecific Antibodies

In another embodiment of the present invention, the inventor has engineered humanized bispecific antibodies. The term “bispecific” means that the molecule is able to specifically bind to at least two distinct moieties (e.g., antigen binding sites). Typically, a bispecific molecule comprises two different binding sites, each of which is specific for a different moiety (e.g., antigen). In certain embodiments, the bispecific molecule is capable of simultaneously binding two moieties, particularly two moieties expressed on two distinct cells (e.g., a tumor cell and a T cell). The bispecific antibodies described herein are capable of binding a target antigen and a T cell antigen. The term “bispecific antibody”, “bispecific T cell engager” and “BTE” are used interchangeable herein and refer to the antibody molecules that are capable of binding two distinct antigens at the same time. Particularly, the bispecific antibodies are capable of binding to the surface of tumor cells and T cells simultaneously, allowing for activation of the T cells, and the targeting killing of tumor cells bound to the bispecific antibody.

The bispecific antibodies described herein comprise or consist of two single-chain variable fragments (scFvs) connected by a flexible linker. One of the scFvs is directed to a target associated antigen, in this particular invention, to IL13Rα2 which is found on cancer cells, particularly glioblastoma. The other scFv is capable of binding to an activating T cell antigen, in this particular embodiment, a CD3 ε that is expressed on T cells. The bispecific antibody by binding CD3 engage tumor infiltrating lymphocytes (TILs) and cancer cells in an MHC independent manner and are, therefore, unaffected by MHC downregulation that occurs in some cancers, for example, in glioblastoma (GBM) cells. The specificity of bispecific antibody's tumor antigen-directed scFv is imperative to harness the full therapeutic potential of the recombinant molecule. BTE anti-cancer activity requires BTE binding with malignant cancer and immune cells simultaneously; as has been demonstrated in the art, single-arm binding to a tumor antigen or CD3F is therapeutically ineffective.

An “activating T cell antigen” as used herein refers to an antigenic determinant expressed on the surface of a T lymphocyte, particularly a cytotoxic T lymphocyte, which is capable of inducing T cell activation upon interaction with an antigen binding molecule. Specifically, interaction of an antigen binding molecule with an activating T cell antigen may induce T cell activation by triggering the signaling cascade of the T cell receptor complex. In a particular embodiment the activating T cell antigen is CD3.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating bispecific antigen binding molecules of the invention are capable of inducing T cell activation. Suitable assays to measure T cell activation are known in the art described herein.

Expression of IL13Rα2 in GBM 12, GBM 6, and GBM 39 used for toxicity and T cells activation assays are known in the art and have been previously described by the inventor (e.g, Balyasnikova et. al. Characterization and immunotherapeutic implications for a novel antibody targeting interleukin (IL)-13 receptor α2. J Biol Chem. 2012 Aug. 31; 287(36):30215-27. doi: 10.1074/jbc.M112.370015. Epub 2012 Jul. 9. PMID: 22778273; PMCID: PMC3436275, and Pituch et al. Neural stem cells secreting bispecific T cell engager to induce selective antiglioma activity. Proc Natl Acad Sci USA. 2021 Mar. 2; 118(9):e2015800118. doi: 10.1073/pnas.2015800118. PMID: 33627401; PMCID: PMC7936285.)

The “target cell antigen” as used herein refers to an antigen presented on the surface of a target cell, for example a cancer cell or a cell of the tumor stroma. Specifically, in the present invention, the target cell antigen is IL13Rα2, which is found on glioblastoma cells.

In one embodiment, the present disclosure provides an engineered bispecific T cell engager comprising a first single-chain variable fragment (scFv) that binds to CD3 and a second scFv that binds to IL13Rα2 as described herein. In one embodiment, the first scFv comprises: (a) a variable heavy domain (VH) comprising an amino acid sequence of SEQ ID NO:51 or an amino acid sequence with at least 95% sequence similarity to SEQ ID NO:51; (b) a first flexible linker; and (c) a variable light domain (VL) comprising an amino acid sequence of SEQ ID NO:52 or an amino acid sequence with at least 95% sequence similarity to SEQ ID NO:52, and wherein the second scFv comprises: (d) a variable light domain (VL) comprising an amino acid sequence of SEQ ID NO:53 or an amino acid with at least 95% sequence similarity to SEQ ID NO:53; (e) a second linker; and (f) a variable heavy domain (VH) comprising an amino acid sequence of SEQ ID NO:54 or an amino acid with at least 95% sequence similarity to SEQ ID NO:54; and wherein the bispecific antibody comprises from 5′ to 3′: the VH of the first scFv, the VL of the first scFv, the VL of the second scFv, and the VH of the second scFv. Specifically, the orientation of the VH and VL domains and linker between two scFvs is important for the design and proper functioning of the bispecific antibody. Thus, the suitable designed bispecific antibodies have the following orientation: a-CD3 VH -linker-αCD3 VL-linker-humanized VH scFvIL13Rα2- linker humanized scFvIL13Rα2 VL. Suitable polypeptides comprising the bispecific antibodies are found in SEQ ID NO:48, SEQ ID NO:49; SEQ ID NO:56-59. In some embodiments, the engineered bispecific antibodies comprise the first single-chain variable fragment (scFv) that binds to CD3 and second scFv that binds to IL13Rα2 are linked via a third flexible linker. Suitable linkers are known in the art and include those described above for the humanized antibodies, including, peptide sequences of 5-25 amino acids, preferably comprising glycines and serines. Suitable first and second linker include an amino acid sequence of about 10⁻²⁰ amino acids, the amino acids selected from glycine and serine. In one suitable examples, the first and second linker are (Gly₄S)₃ (SEQ ID NO:55). In some aspects, the third linker is a 20-30 amino acid glycine-serine linker, for example, SEQ ID NO:56.

As used herein, the terms “first” and “second” with respect to scFv molecules, linkers, etc. are used for convenience of distinguishing when there is more than one of each type of moiety.

The bispecific antibodies described herein are engineered to be humanized. As discussed above, the humanized antibody against IL13Rα2 may include one or more mutations (e.g., G56A, W100F, or combinations thereof) that improve the stability, binding or both for the bispecific antibodies. In one embodiment, the scFv that binds to IL13Rα2 has a G52A mutation in the V_{H} domain (e.g., X₃ is A in SEQ ID NO:48 or 54). In another embodiment, the bispecific antibodies comprises the D55E mutation in the V_{H} domain (e.g., X₂ is E in SEQ ID NO:48 or 54). In a further embodiment, the bispecific antibody may be a mutation in the VL domain, e.g., W100F (e.g., X₁ in SEQ ID NO:48 or 53).

In some embodiments, the bispecific antibody further comprises a tag. Suitable tags are known in the art and described above, including epitope tags and purifications tags such as, for example, 6-Histidine (His), hemagglutinin (HA), eMyc, GST, Flag tag, etc.

Suitable examples of engineered bispecific antibodies of the present inventions are provided herein and include the polypeptide comprising or consisting of SEQ ID NO:48, SEQ ID NO:49, SEQ ID NO: 57, SEQ ID NO: 58, SEQ ID NO: 59, SEQ ID NO:60, etc. or amino acid sequences having at least 95% sequence identity, at least 98% sequence identity, at least 99% sequence identity, 100% sequence identity to SEQ ID NO:48, 49, 57-60.

In some embodiments, a nucleic acid sequence encoding the bispecific antibody described herein, including vectors, is contemplated. In a further embodiment, a transgenic neural stem cell (NSC) that expresses the bispecific T cell engager described herein is also provided. Neural stem cells (NSCs) have inherent advantages as a cellular carrier of antineoplastic agents to the site of GBM since they are native to the brain. NSCs have demonstrated tropism to brain tumors in several preclinical models. These cells can withstand a harsh oxygen-deprived environment of GBM. NSCs can be used as producers of bispecific targeting the tumor-associated antigen IL13Rα2 and their antitumor activity using in vitro and in vivo models of GBM. In vitro, bispecific antibodies show significant antitumor activity when used in co-cultures that include T cells harvested from patients' blood and tumor tissue. In vivo, NSCs modified for bispecific antibody synthesis migrate to a tumor in animal subjects' brains while functioning as intra- and peritumoral bispecific antibody producers.

The terms “stability” and “stable” in the context of antibodies and bispecific antibodies describe the resistance of the antibodies or their fragments with respect to aggregation, degradation or fragmentation under the given conditions relating to their production, preparation, storage, use or transport. “Stable” formulations according to the present invention retain their biological activity under the given production, preparation, transport, use and storage conditions.

As described herein, the humanized antibodies and the engineered humanized bispecific antibodies are specific to two different moieties and display a higher binding affinity that the parent mouse antibody or derived chimeric antibodies, and shown in the examples. By “specific binding” we mean that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed on a BIAcore instrument) (Liljeblad et al, Glyco J 17, 323-329 (2000)), and traditional binding assays (Heeley, Endocr Res 28, 217-229 (2002)).

In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of <1 μM, <100 nM, <10 nM, <1 nM, <0.1 nM, <0.01 nM, or <0.001 nM (e.g. 10^{~8} M or less, e.g. from 10^{~8} M to 10⁻¹³ M, e.g., from 10⁻⁹ M to 10⁻¹³ M).

### Additional Compositions:

The present invention provides compositions comprising the humanized antibodies and humanized bispecific antibodies disclosed herein and a pharmaceutically acceptable carrier. The pharmaceutically acceptable carrier should be selected based upon the desired route of administration. For example, the humanized antibodies may be provided in combination with liposomes, nanoparticles or other analogous carriers loaded with a pharmaceutically active compound. Methods of preparing such compositions are known in the art (see, for example, Cancer Research (2000) 60, 6942-6949 and Analytical Chemistry News &Features (1998) pp. 322A-327A).

“Pharmaceutically acceptable” carriers are known in the art and include, but are not limited to, for example, suitable diluents, preservatives, solubilizers, emulsifiers, liposomes, nanoparticles, and adjuvants. Pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media.

The compositions of the present invention may further include liquids or lyophilized or otherwise dried formulations and may include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, milamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

In some embodiments, the compositions are provided in lyophilized form and rehydrated with sterile water or saline solution before administration. Alternatively, the compositions may be provided in a sterile solution of known concentration. Further, the compositions may be added to an infusion bag containing 0.9% sodium chloride, USP and in some cases, administered in a dosage of from 0.5 to 15 mg/kg of body weight.

The compositions may also be prepared in unit dosaged forms for administration to a subject. The amount and timing of administration are at the discretion of the treating clinician to achieve the desired outcome. The humanized antibodies can be formulated for systemic or local (e.g., intravenous, intrathecal, intra-cranial) administration. In one example, the antibody is formulated for parenteral administration, such as intravenous administration.

In another aspect, the present invention provides nucleic acid sequences encoding the humanized antibodies disclosed herein. In some embodiments, the nucleic acid sequences comprise conservative or inconsequential substitutions or deletions.

In another aspect, the present invention provides vectors comprising the nucleic acid sequences that encode the humanized antibodies or engineered humanized bispecific antibodies disclosed herein. The vectors may be an expression vector comprising an expression cassette encoding the humanized antibodies or engineered humanized bispecific antibodies, or encoding the agent in addition to the humanized antibodies or bispecific antibodies. For example, a nucleic acid sequence encoding an antibody may be under the control of a heterologous promoter, allowing for the regulation of the transcription of said nucleic acid sequence in a cell. Said nucleic acid sequence can also be linked to appropriate control sequences to allow for regulation of its translation in a cell. Suitable vectors further include, for example, viral vectors, that allow for the transduction and expression of the engineered humanized antibodies and humanized bispecific antibodies described herein.

### Methods:

In another aspect, the present invention provides methods of treating an IL13Rα2-expressing cancer in a subject. The methods comprise administering a therapeutically effective amount of the humanized antibodies, bispecific antibodies or compositions disclosed herein to treat the cancer.

Importantly, while IL13Rα2 is expressed by many cancer cells, it is not expressed by healthy tissues, with the exception of testes. Thus, the humanized antibodies or bispecific antibodies disclosed herein can be used as a targeting moiety to deliver therapeutics to cancer cells with low risk of off-target toxicity. In some embodiments, the methods further comprise determining whether IL13Rα2 is expressed on a cancer cell in the subject, and the humanized antibody or composition is only administered to the subject if IL13Rα2 expression is detected.

To determine whether IL13Rα2 is expressed on a cancer cell in the subject, a tissue sample or biopsy may be collected and analyzed using any standard method used to detect gene or protein expression. Suitable methods include, without limitation, Northern blot, western blot, in situ hybridization, immunobistochemistry, immunocytochemistry, reverse transcription polymerase chain reaction, microarray, RNA sequencing, and the like.

While the focus of the inventor's research is the treatment of glioblastoma, IL13Rα2 overexpression has been observed in a variety of tumor types, including colorectal cancer, renal cell carcinoma, pancreatic cancer, melanoma, head and neck cancer, mesothelioma, breast cancer, lung cancer, osteosarcoma, ovarian cancer, and metastases thereof (see, e.g., Cancers 2020, 12(2), 500). Any cancer type that expresses IL13Rα2 can be treated using the methods of the present invention. In a preferred embodiment, the cancer is glioblastoma.

The methods of the present invention may comprise an immunotherapy treatment, such as a vaccination, immune-checkpoint blockade, antibody-drug conjugates, tumor-infiltrating lymphocytes (TILs), bi-specific T-cell engagers (BTEs), or T cells modified with chimeric antigen receptors (CARs). For example, the humanized antibodies disclosed herein may be incorporated into reagents such as BTEs or CARs to give them the ability to target IL13Rα2-expressing cancer cells. Alternatively, the antibodies may be fused with the signaling domain of a T cell signaling protein (e.g., 4-1BB or CD3ξ) or a peptide modulator of T cell activation (e.g., IL-15 or IL-15Ra) to activate immune cell activity at the site of a tumor.

In addition, the antibodies may be used as targeting moieties used to deliver other therapeutics to cancer cells. To this end, the antibody may be conjugated to a therapeutic agent, as discussed above. Exemplary therapeutic agents for the treatment of cancer include cytotoxic and chemotherapeutic agents, such as platinum coordination compounds (e.g., cisplatin), topoisomerase inhibitors (e.g., topotecan, irinotecan and 9-amino-camptothecin), antibiotics (e.g., doxorubicin, mitomycin, bleomycin, daunorubicin and streptozocin), antimitotic alkaloids (e.g., vinblastine, vincristine, vindesine, Taxol and vinorelbine), and difluoronucleosides (e.g., 2′-deoxy-2′,2′-difluorocytidine hydrochloride). However, the antibodies of the present invention may be used to target any chemotherapeutic agent to ILI3Rα2-expresing cancer cells.

In one embodiment, the disclosed antibodies may be used as a targeting moiety by being linked to a peptide providing a second function, e.g., an effector function, such as a T cell signaling domain involved in T cell activation, a peptide that affects or modulates an immunological response to cancer cells, or an enzymatic component of a labeling system that results in a CAR encoded by a polynucleotide according to the disclosure. Exemplary conjugates include an anti-IL13Rα2 scFv linked to a hinge, a transmembrane domain, and an effector compound or domain, e.g., CD28, CD3ξ, CD134 (OX40), CD137 (41BB), ICOS, CD40, CD27, or Myd88, thereby yielding a CAR.

In some embodiments, the humanized antibody (being used as a targeting moiety) is used in a conjugate, wherein the conjugate further comprises an effector domain. As used herein, the term “effector domain” refers to a portion of a conjugate that effects a desired biological function.

In some embodiments, the effector domain is an apoptosis tag, for example, a TRAIL protein, or a portion thereof. An apoptosis tag is a tag that causes the IL13Rα2-expressing cell to apoptose.

In some embodiments, the effector domain is a label. Suitable labels include, for example, but not limited to, a radiolabel, a fluorescent label, an enzyme that catalyzes a calorimetric or fluorometric reaction, an enzymatic substrate, a solid matrix, biotin or avidin.

In another embodiment, the effector domain identifies or locates IL13Rα2-expressing cells. For example, the effector domain may be a diagnostic agent, e.g., a radiolabel, a fluorescent label, an enzyme (e.g., that catalyzes a calorimetric or fluorometric reaction), a substrate, a solid matrix, or a carrier (e.g., biotin or avidin). The diagnostic agent in some aspects is an imaging agent. Many appropriate imaging agents are known in the art, as are methods of attaching the labeling agents to the peptides of the invention (see, e.g., U.S. Pat. Nos. 4,965,392; 4,472,509; 5,021,236; and 5,037,630; each incorporated herein by reference). The imaging agents are administered to a subject in a pharmaceutically acceptable carrier, and allowed to accumulate at a target site having the lymphatic endothelial cells. This imaging agent then serves as a contrast reagent for X-ray, magnetic resonance, positron emission tomography, single photon emission computed tomography (SPECT), or sonographic or scintigraphic imaging of the target site. Imaging may occur in vitro or in vivo, for example, imaging may be performed in vitro where tissue from the subject is obtained through a biopsy, and the presence of IL13Rα2 positive cells is determined with the aid of the imaging agents described herein in combination with histochemical techniques for preparing and fixing tissues. Paramagnetic ions useful in the imaging agents of the invention include for example chromium (III), manganese (II), iron (III), iron (II), cobalt (II), nickel (II) copper (II), neodymium (III), samarium (III), ytterbium(III), gadolinium (III), vanadium (II), terbium (III), dysprosium (III), holmium (III) and erbium (III). Ions useful for X-ray imaging include, but are not limited to, lanthanum (III), gold (III), lead (II) and particularly bismuth (III).

Radioisotopes for diagnostic applications include for example, ²¹¹astatine, ¹⁴carbon, ⁵¹chromium, ³⁶chlorine, ⁵⁷cobalt, ⁶⁷copper, ¹⁵²europium, ⁶⁷gallium, ³hydrogen, ¹²³iodine, ¹²⁵iodine, ¹¹¹indium, ⁵⁹iron, ³²phosphorus, ¹⁸⁶rhenium, ⁷⁵selenium, ³⁵sulphu r, ⁹⁹mtechnicium, ⁹⁰yttrium, and ⁸⁹zirconium.

In another embodiment, the effector domain may be one that alters the physico-chemical characteristics of the conjugate, e.g., an effector that confers increased solubility and/or stability and/or half-life, resistance to proteolytic cleavage, modulation of clearance. In exemplary aspects, the effector domain is a polymer, a carbohydrate, or a lipid. The polymer may be branched or unbranched. The polymer may be of any molecular weight. The polymer in some embodiments has an average molecular weight of between about 2 kDa to about 100 kDa (the term “about” indicating that in preparations of a water-soluble polymer, some molecules will weigh more, some less, than the stated molecular weight). The average molecular weight of the polymer is in some aspects between about 5 kDa and about 50 kDa, between about 12 kDa to about 40 kDa or between about 20 kDa to about 35 kDa. In some embodiments, the polymer is modified to have a single reactive group, such as an active ester for acylation or an aldehyde for alkylation, so that the degree of polymerization may be controlled. The polymer in some embodiments is water-soluble so that the protein to which it is attached does not precipitate in an aqueous environment, such as a physiological environment. In some embodiments when, for example, the composition is used for therapeutic use, the polymer is pharmaceutically acceptable. Additionally, in some aspects, the polymer is a mixture of polymers, e.g., a co-polymer, a block co-polymer. In some embodiments, the polymer is selected from the group consisting of: polyamides, polycarbonates, polyalkylenes and derivatives thereof, including polyalkylene glycols, polyalkylene oxides, polyalkylene terepthalates, polymers of acrylic and methacrylic esters, including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), polyvinyl polymers including polyvinyl alcohols, polyvinyl ethers, polyvinyl esters, polyvinyl halides, poly(vinyl acetate), and polyvinylpyrrolidone, polyglycolides, polysiloxanes, polyurethanes and co-polymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt, polypropylene, polyethylenes including poly(ethylene glycol), poly(ethylene oxide), and poly(ethylene terephthalate), and polystyrene. In some aspects, the polymer is a biodegradable polymer, including a synthetic biodegradable polymer (e.g., polymers of lactic acid and glycolic acid, polyanhydrides, poly(ortho)esters, polyurethanes, poly(butic acid), poly(valeric acid), and poly(lactide-cocaprolactone)), and a natural biodegradable polymer (e.g., alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins (e.g., zein and other prolamrines and hydrophobic proteins)), as well as any copolymer or mixture thereof. In general, these materials degrade either by enzymatic hydrolysis or exposure to water in vivo, by surface or bulk erosion. In some aspects, the polymer is a bioadhesive polymer, such as a bioerodible hydrogel described by I-1. S. Sawhney, C. P. Pathak and J. A. Hubbell in Macromolecules, 1993, 26., 581-587, the teachings of which are incorporated herein, polyhyaluronic acids, casein, gelatin, glutin, polyanhydrides, polyacrylic acid, alginate, chitosan, poly(methyl methacrylates), poly(ethyl rnethacrylates), poly(butyimethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). In some embodiments, the polymer is a water-soluble polymer or a hydrophilic polynmer. Suitable water-soluble polymers are known in the art and include, for exarnple, polyvinylpyrrolidone, hydroxypropyl cellulose (HPC; Klucel), hydroxypropyl methylcellulose (HPMC; Methocel), nitrocellulose, hydroxypropyl ethylcellulose, hydroxypropyl butylcellulose, hydroxypropyl pentylcellulose, methyl cellulose, ethylcellulose (Ethocel), hydroxyethyl cellulose, various alkyl celluloses and hydroxyalkyl celluloses, various cellulose ethers, cellulose acetate, carboxymethyl cellulose, sodium carboxymethyl cellulose, calcium carboxymethyl cellulose, vinyl acetate/crotonic acid copolymers, poly-hydroxyalkyl methacrylate, hydroxymethyl methacrylate, methacrylic acid copolymers, polymethacrylic acid, polymethylmethacrylate, maleic anhydride/methyl vinyl ether copolymers, poly vinyl alcohol, sodium and calcium polyacrylic acid, polyacrylic acid, acidic carboxy polymers, carboxypolymethylene, carboxyvinyl polymers, polyoxyethylene polyoxypropylene copolymer, polymethylvinylether co-maleic anhydride, carboxymethylamide, potassium methacrylate divinylbenzene co-polymer, polyoxyethyleneglycols, polyethylene oxide, and derivatives, salts, and combinations thereof. In some aspects, the water-soluble polymers or mixtures thereof include, but are not limited to, N-linked or O-linked carbohydrates, sugars, phosphates, carbohydrates; sugars; phosphates; polyethylene glycol (PEG) (including the forms of PEG that have been used to derivatize proteins, including mono-(C1-C 10) alkoxy- or aryloxy-polyethylene glycol); monomethoxy-polyethylene glycol; dextran (such as low molecular weight dextran of, for example, about 6 kD), cellulose; other carbohydrate-based polymers, poly-(N-vinyl pyrrolidone), polyethylene glycol, propylene glycol homopolymers, a polypropylene oxide/ethylene oxide co-polymer, polyoxyethylated polyols (e.g., glycerol) and polyvinyl alcohol. Also encompassed by the disclosure are bifunctional crosslinking molecules that may be used to prepare covalently attached multimers. A particularly preferred water-soluble polymer for use herein is polyethylene glycol (PEG). As used herein, polyethylene glycol is meant to encompass any of the forms of PEG that can be used to derivatize other proteins, such as mono-(C1-C10) alkoxy- or aryloxy-polyethylene glycol. PEG is a linear or branched neutral polyether, available in a broad range of molecular weights, and is soluble in water and most organic solvents. PEG is effective at excluding other polymers or peptides when present in water, primarily through its high dynamic chain mobility and hydrophobic nature, thus creating a water shell or hydration sphere when attached to other proteins or polymer surfaces. PEG is nontoxic, non-imimunogenic, and approved by the Food and Drug Administration for internal consumption. Proteins or enzymes when conjugated to PEG have demonstrated bioactivity, non-antigenic properties, and decreased clearance rates when administered in animals. F. M. Veronese et al., Preparation and Properties of Monomethoxypoly(ethylene glycol)-modified Enzymes for Therapeutic Applications, in J. M.

Harris ed., Poly(Ethylene Glycol) Chemistry-Biotechnical and Biomedical Applications, 127-36, 1992, incorporated herein by reference. PEG in thought to prevent recognition by the immune system. In addition, PEG has been widely used in surface modification procedures to decrease protein adsorption and improve blood compatibility. S. W. Kim et al., Ann. N.Y. Acad. Sci. 516: 116-30 1987; Jacobs et al., Artif Organs 12: 500-501, 1988; Park et al., J Poly. Sci, Part A 29:1725-31, 1991, each incorporated herein by reference in its entirety. Hydrophobic polymer surfaces, such as polyurethanes and polystyrene, can be modifed by the grafting of PEG (MW 3,400) and employed as nonthrombogenic surfaces. Surface properties (contact angle) can be more consistent with hydrophilic surfaces, due to the hydrating effect of PEG. More importantly, protein (albumin and other plasma, proteins) adsorption can be greatly reduced, resulting from the high chain motility, hydration sphere, and protein exclusion properties of PEG. PEG (M/W 3,400) was determined as an optimal size in surface immobilization studies, Park et al., J. Biomed. Mat. Res. 26:739-45, 1992, while PEG (MW 5,000) was most beneficial in decreasing protein antigenicity. F. M. Veronese et al., In J. M. Harris, et al., Poly(Ethylene Glycol) Chemistry-Biotechnical and Biomedical Applications, 127-36. Methods for preparing pegylated polypeptides (e.g., humanized antibody) may comprise the steps of (a) reacting the polypeptide with polyethylene glycol (such as a reactive ester or aldehyde derivative of PEG) under conditions whereby the humanized antibody polypeptide becomes attached to one or more PEG groups, and (b) obtaining the reaction product(s). In general, the optimal reaction conditions for the acylation reactions will be determined based on known parameters and the desired result. For example, the larger the ratio of PEG:protein, the greater the percentage of poly-pegylated product. In some embodiments, the humanized antibody will have a single PEG moiety at the N-terminus. See U.S. Pat. No. 8,234,784, incorporated by reference herein.

In some embodiments, the effector domain is a carbohydrate. In some embodiments, the carbohydrate is a monosaccharide (e.g., glucose, galactose, fructose), a disaccharitde (e.g., sucrose, lactose, maltose), an oligosaccharide (e.g., raffinose, stachyose), a polysaccharide (e.g., a starch, amylase, amylopectin, cellulose, chitin, callose, laminarin, xylan, mannan, fucoidan, or galactomannan). In some embodiments, the effector domain is a lipid. The lipid, in some embodiments, is a fatty acid, eicosanoid, prostaglandin, leukotriene, thromboxane, N-acyl ethanolamine, glycerolipid (e.g., mono-, di-, tri-substituted glycerols), glycerophospholipid (e.g., phosphatidylcholine, phosphatidylinositol, phosphatidylethanolamine, phosphatidylserine), sphingolipid (e.g., sphingosine, ceramide), sterol lipid (e.g., steroid, cholesterol), prenol lipid, saccharolipid, or a polyketide, oil, wax, cholesterol, sterol, fat-soluble vitamin, monoglyceride, diglyceride, triglyceride, or a phospholipid.

In another exemplary embodiment, the effector domain is a lethal domain that confers lethality, such that when the conjugate is localized to a cell expressing IL13R.2, e.g., a tumor cell expressing IL13Rα2. The effector domain confers upon the conjugate the ability to kill an IL13Rα2-expressing cell once the humani zed antibody has found and bound to its IL13Rα2 target.

In exemplary embodiments, the effector domain is a cytotoxin (also referred to herein as a “cytotoxic agent”). The cytotoxic agent is any molecule (chemical or biochemical) which is toxic to a cell, in some embodiments, the cytotoxic agent is a chemotherapeutic agent. Chemotherapeutic agents are known in the art and include, but are not limited to, platinum coordination compounds, topoisonierase inhibitors, antibiotics, antimitotic alkaloids and difluoronucleosides, as described in U.S. Pat. No. 6,630,124. In some embodiments, the chemotherapeutic agent is a platinum coordination compound. The term “platinum coordination compound” refers to any tumor cell growth-inhibiting platinum coordination compound that provides the platinum in the form of an ion. In some embodiments, the platinum coordination compound is cis-diamminediaquoplatinum (II)-ion; chloro(diethylenetriamine)-platinum(II)chloride; dichloro(ethylenediamine)-platinum(II), diammine(1, 1-cyclobutanedicarboxylato) platinum(II) (carboplatin), spiroplatin; iproplatin; diammine(2-ethylmalonato)-platinum(II); ethylenediaminemalonatoplatinum(II); aqua(1,2-diaminodyclohexanie)-sulfatosplatinum(II); (1,2-dianinocyclohexane)mnalonatoplatinum(II); (4-caroxyphthalato)(1,2-dianinocyclohexane)platinum(II); (1,2-diaminocyclobexane)-(isocitrato)platinum(II); (1,2-diaminocyclohexane)cis(pyruvato)platinum(II); (1,2-diaminocyclohexane)oxalatoplatinum(II); ornaplatin; or tetraplatin. In some embodiments, cisplatin is the platinum coordination corpound employed in the compositions and methods of the present invention. Cisplatin is commercially available tinder the name PLATINOL^{T}m from Bristol Myers-Squibb Corporation and is available as a powder for constitution with water, sterile saline or other suitable vehicle. Other platinum coordination compounds suitable for use in the present invention are known and are available commercially and/or can be prepared by conventional techniques. Cisplatin, or cis-dichlorodiammineplatinum II, has been used successfully for many years as a chenotherapeutic agent in the treatment of various human solid malignant tumors. More recently, other diamino-platinur complexes have also shown efficacy as chemotherapeutic agents in the treatment of various human, solid, malignant tumors. Such diamino-platinum complexes include, but are not limited to, spiroplatinum and carboplatinumin. Although cisplatin and other diamino-platinum complexes have been widely used as chemotherapeutic agents in humans, they have had to be delivered at high dosage levels that can lead to toxicity problems such as kidney damage.

In some embodiments, the chemotherapeutic agent is a topoisomerase inhibitor. Topoisomerases are enzymes that are capable of altering DNA topology in eukaryotic cells. They are critical for cellular functions and cell proliferation. Generally, there are two classes of topoisomerases in eukaryotic cells, type I and type II. Topoisoirerase I is a monomeric enzyme of approximately 100,000 molecular weight. The enzyme binds to DNA and introduces a transient single-strand break, unwinds the double helix (or allows it to unwind), and subsequently reseals the break before dissociating from the DNA strand. Various topoisomerase inhibitors have recently shown clinical efficacy in the treatment of humans afflicted with ovarian cancer, esophageal cancer or non-small cell lung carcinoma. In some aspects, the topoisorerase inhibitor is camptothecin or a camptothecin analog. Camptothecin is a water-insoluble, cytotoxic alkaloid produced by Camptotheca accuminata trees indigenous to China and Nothapodytes foetida trees indigenous to India. Camptothecin exhibits tumor cell growth-inhibiting activity against a number of tumor cells. Compounds of the carnptothecin analog class are typically specific inhibitors of DNA topoisomerase I. By the term “inhibitor of topoisomerase” is meant any tumor cell growth-inhibiting compound that is structurally related to camptothecin. Compounds of the camptothecin analog class include, but are not limited to; topotecan, irinotecan and 9-amino-camptothecin. In additional embodiments, the cytotoxic agent is any tumor cell growth-inhibiting camptothecin analog claimed or described in U.S. Pat. No. 5,004,758; European Patent Application Number 88311366.4 (Publication Number EP 0 321 122); U.S. Pat. No. 4,604,463; European Patent Application Publication Number EP 0 137 145; U.S. Pat. No. 4,473,692; European Patent Application Publication Number EP 0 074 256, U.S. Pat. No. 4,545,880; European Patent Application Publication Number EP 0 074 256; European Patent Application Publication Niumber EP 0 088 642; Wani et al., J. Med. Chem., 29., 2358-2363 (1986); and Nitta et al., Proc. 14th International Congr. Chemotherapy, Kyoto, 1985, Tokyo Press, Anticancer Section 1, p. 28-30. In particular, the disclosure contemplates a compound called CPT-11. CPT-II is a camptothecin analog with a 4-(piperidino)-piperidine side chain joined through a carbamate linkage at C-10 of 10-hydroxy-7-ethyl camptothecin. CPT-11 is currently undergoing human clinical trials and is also referred to as irinotecan; Wani et al, J. Med. Chem., 23, 554 (1980); Wani et, al., J. Med. Chem., 30, 1774 (1987); U.S. Pat. No. 4,342,776; European Patent Application Publication Number EP 418 099; U.S. Pat. No. 4,513,138; European Patent Application Publication Nunber EP 0 074 770; U.S. Pat. No. 4,399,276; European Patent Application Publication Number 0 056 692; the entire disclosure of each of which is hereby incorporated by reference. All of the above-listed compounds of the camptothecin analog class are available commercially and/or can be prepared by conventional techniques including those described in the above-listed references. The topoisomerase inhibitor may be selected from the group consisting of topotecan, irinotecan and 9-aminocamptothecin.

The preparation of numerous compounds of the camptothecin analog class (including pharmaceutically acceptable salts, hydrates and solvates thereof) as well as the preparation of oral and parenteral pharmaceutical compositions comprising such a compound of the camptothecin analog class and an inert, pharmaceutically acceptable carrier or diluent, is extensively described in U.S. Pat. No. 5,004,758; and European Patent Application Number 88311366.4 (Publication Number EP 0 321 122) the teachings of each of which are incorporated herein by reference in its entirety.

In still another embodiment, the chemotherapeutic agent is an antibiotic compound. Suitable antibiotics include, but are not limited to, doxorubicin, mitomycin, bleomycin, daunorubicin and streptozocin. In some embodiments, the chemotherapeutic agent is an antimitotic alkaloid. In general, antimitotic alkaloids can be extracted from Cantharanthus roseus, and have been shown to be efficacious as anticancer chemotherapy agents. A great number of semi-synthetic derivatives have been studied both chemically and pharmacologically (see, C. Van Tellingen et al, Anticancer Research, 12, 1699-1716 (1992)) The antimitotic alkaloids of the present invention include, but are not limited to, vinblastine, vincristine, vindesine, Taxol and vinorelbine. The latter two antimitotic alkaloids are commercially available from Eli Lilly and Company, and Pierre Fabre Laboratories, respectively (see, U.S. Pat. No. 5,620,985). In one aspect of the disclosure, the antimitotic alkaloid is vinorelbine.

In another embodiment, the chemotherapeutic agent is a difluoronucleoside. 2′-deoxy-2′,2′-difluoronucleosides are known in the art as having antiviral activity. Such compounds are disclosed and taught in U.S. Pat. Nos. 4,526,988 and 4,808,614. European Patent Application Publication 184,365 discloses that these same difluoronucleosides have oncolytic activity. In certain specific aspects, the 2′-deoxy-2′,2′-difluoronucleoside used in the compositions and methods of the disclosure is 2′-deoxy-2′,2′-difluorocytidine hydrochloride, also known as gemcitabine hydrochloride. Gemcitabine is commercially available or can be synthesized in a multi-step process as disclosed in U.S. Pat. Nos. 4,526,988, 4,808,614 and 5,223,608, the teachings of each of which are incorporated herein by reference in its entirety.

In another exemplary embodiment, the effector domain is an Fc domain of IgG or other immunoglobulin. For substituents such as an Fe region of human IgG, the fusion can be fused directly to humanized antibody described herein or fused through an intervening sequence. For example, a human IgG hinge, CH2 and CH3 region may be fused at either the N-terminus or C-terminus of the humanized antibody to attach the Fe region. The resulting Fe-fusion agent enables purification via a Protein A affinity column (Pierce, Rockford, Ill.). Peptide and proteins fused to an Fe region can exhibit a substantially greater half-life in vivo than the unfused counterpart. A fusion to an Fc region allows for dimerization/multimerization of the fusion polypeptide. The Fe region may be a naturally occurring Fe region, or may be modified for superior characteristics, e.g., therapeutic qualities, circulation time, reduced aggregation. As noted above, in some embodiments, the humanized antibodies are conjugated, e.g., fused to an immunoglobulin or portion thereof (e.g., variable region, CDR, or Fe region). Known types of immunoglobulins (Ig) include IgG, IgA, IgE, IgD or IgM. The Fc region is a C-terminal region of an Ig heavy chain, which is responsible for binding to Fe receptors that carry out activities such as recycling (which results in prolonged half-life), antibody dependent cell-mediated cytotoxicity (ADCC), and complement dependent cytotoxicity (CDC).

For example, according to some definitions the human IgG heavy chain Fc region stretches from Cys226 to the C-terminus of the heavy chain. The “hinge region” generally extends from Glu216 to Pro230 of human IgG1 (hinge regions of other IgG isotypes may be aligned with the IgG1 sequence by aligning the cysteines involved in cysteine bonding). The Fc region of an IgG includes two constant domains, CH2 and CH3. The CR2 domain of a human IgG Fe region usually extends from amino acids 231 to amino acid 341. The CH3 domain of a human IgG Fc region usually extends from amino acids 342 to 447. References made to amino acid numbering of immunoglobulins or immunoglobulin fragments, or regions, are all based on Kabat et al. 1991, Sequences of Proteins of Immunological Interest, U S. Department of Public Health, Bethesda, Md., incorporated herein by reference. In related embodiments, the Fc region may comprise one or more native or modified constant regions from an immunoglobulin heavy chain, other than CH1, for example, the CH2 and CH3 regions of IgG and IgA, or the CH3 and CH4 regions of IgE.

Suitable conjugate moieties include portions of immunoglobulin sequence that include the FcRn binding site. FcRn, a salvage receptor, is responsible for recycling immunoglobulins and returning them to circulation in the blood. The region of the Fe portion of IgG that binds to the FcRn receptor has been described based on X-ray cry stallography (Burmeister et al. 1994, Nature 372:379). The major contact area of the Fc with the FcRn is near the junction of the CH2 and CH3 domains. Fc-FcRn contacts are all within a single Ig heavy chain. The major contact sites include amino acid residues 248, 250-257, 272, 285, 288, 290-291, 308-311, and 314 of the CH2 domain and amino acid residues 385-387, 428, and 433-436 of the C113 domain.

Some conjugate moieties may or may not include FcγR binding site(s). FcγR are responsible for antibody-dependent cell-mediated cytotoxicity (ADCC) and complement-dependent cytotoxicity (CDC). Examples of positions within the Fc region that make a direct contact with FcγR are amino acids 234-239 (lower hinge region), amino acids 265-269 (B/C loop), amino acids 297-299 (C′/E loop), and amino acids 327-332 (F/G) loop (Sondermann et al., Nature 406: 267-273, 2000). The lower hinge region of IgE has also been implicated in the FcRI binding (Henry, et al., Biochemistry 36, 15568-15578, 1997). Residues involved in IgA receptor binding are described in Lewis et al., (J Immunol. 175:6694-701, 2005). Amino acid residues involved in IgE receptor binding are described in Savers et al. (J Biol Chem. 279(34):35320-5, 2004).

Amino acid modifications may be made to the Fe region of an immunoglobulin. Such variant Fc regions comprise at least one amino acid modification in the CH3 domain of the Fc region (residues 342-447) and/or at least one amino acid modification in the CH2 domain of the Fe region (residues 231-341). Mutations believed to impart an increased affinity for FcRn include T256A, T307A, E380A, and N434A (Shields et al. 2001, J. Biol. Chem. 276:6591). Other mutations may reduce binding of the FEc region to FeγRI, FcγRIIA, FcγRII3, and/or FcγRIIIA without significantly reducing affinity for FeRn. For example, substitution of the Asn at position 297 of the Fe region with Ala or another amino acid removes a highly conserved N-glycosylation site and may result in reduced immunogenicity with concomitant prolonged half-life of the Fe region, as well as reduced binding to FcγRs (Routledge et al. 1995, Transplantation 60:847; Friend et al. 1999, Transplantation 68:1632; Shields et al. 1995, J. Biol. Chem 276:6591). Amino acid modifications at positions 233-236 of IgG1 have been made that reduce binding to FcγRs (Ward and Ghetie 1995, Therapeutic Immunology 2:77 and Armour et al. 1999, Eur. 1. Immunol. 29:2613). Some exemplary amino acid substitutions are described in U.S. Pat. Nos. 7,355,008 and 7,381,408, each of which is incorporated by reference herein in its entirety.

In some embodiments, the humanized antibody is fused to alkaline phosphatase (AP). Methods for making Fe or AP fusion agents are provided in WO 02/060950.

In another exemplary embodiment, the effector domain is a T-cell signaling domain. For example, the conjugate is a chimeric antigen receptor (CAR). Chimeric antigen receptors (CARs) are engineered transmembrane proteins that combine the specificity of an antigen-specific antibody with a T-cell receptor's function. In general, CARs comprise an ectodomain, a transmembrane domain, and an endodomain. The ectodomain of a CAR in some embodiments may comprise an antigen recognition region, which may be the humanized scFV described herein. The ectodomain also in some embodiments comprises a signal peptide that directs the nascent protein into the endoplasmic reticulum. In exemplary embodiments, the ectodomain comprises a spacer that links the humanized antibody described herein to the transmembrane domain. The transmembrane (1TM) domain is the portion of the CAR that traverses the cell membrane. In exemplary embodiments, the TM domain comprises a hydrophobic alpha helix. In exemplary embodiments, the TM domain comprises all or a portion of the TM domain of CD28. In exemplary embodiments, the TM domain comprises all or a portion of the TM domain of CD8α. The endodomain of a CAR comprises one or more signaling domains In exemplary embodiments, the endodomain comprises the zeta chain of CD3, which comprises three copies of the Immunoreceptor Tyrosine-based Activation Motif (ITAM). An ITAM generally comprises a Tyr residue separated by two amino acids from a Leu or Ile. In the case of immune cell receptors, e.g., the T cell receptor and the B cell receptor, the ITAMs occur in multiples (at least two) and each ITAM is separated from another by 6-8 amino acids. The endodomain of CARs may also comprises additional signaling domains, e.g., portions of proteins that are important for downstream signal transduction. In exemplary embodiments, the endodomain comprises signaling domains from one or more of CD28, 41BB or 4-11B (CD137), ICOS, CD27, CD40, OX40 (CD134), or Myd88. Sequences encoding signaling domains of such proteins are provided herein as SEQ ID NOs: 39-42, 68-79, 81, and 83. Methods of making CARs, expressing them in cells, e.g., T-cells, and utilizing the CAR-expressing T-cells in therapy, are known in the art. See, e.g., International Patent Application Publication Nos. WO2014/208760, WO2014/190273, WO2014/186469, WO2014/184143, WO2014180306, WO2014/179759, WO2014/153270, U.S. Application Publication Nos. US20140369977, US20140322212, US20140322275, US20140322183, US20140301993, US20140286973, US20140271582, US20140271635, US20140274909, European Application Publication No. 2814846, each of which are incorporated by reference in their entirety.

In exemplary embodiments, the conjugate of the disclosure is an IL13Rα2-specific chimeric antigen receptor (CAR) comprising the humanized antibody described herein, a hinge region, and an endodomain comprising a signaling domain of a CD)3 zeta chain and a signaling domain of CD28, CD134, and/or CD137. In exemplary embodiments, the CAR comprises (A) humanized antibody described herein, (B) a hinge region; and (C) an endodomain comprising a signaling domain of a C)3 zeta chain and a signaling domain of CD28, CD134, and/or CD137. In exemplary embodiments, the CAR further comprises a transmembrane (TM) domain based on the TM domain of CD8. In exemplary embodiments, the endodomain further comprises a signaling domain of one or more of: CD137, CD134, CD27, CD40, ICOS, and Myd88.

In exemplary embodiments, the CAR comprises (A) the humanized antibody described herein; (B) a hinge region; (C) an endodomain comprising a signaling domain of a CD3 zeta chain and a signaling domain of CD28 and at least one other signaling domain. In one embodiment, the CAR comprises an endodomain comprising a signaling domain of 41BB (CD 137). In another embodiment, the CAR comprises an endodomain comprising a signaling domain of OX40 (CD134). In another embodiment, the CD137 signaling is N-terminal to a C1D3 zeta chain signaling chain. In another embodiment, the CAR comprises (A) the humanized antibody described herein, (B) a hinge region; (C) a transmembrane domain of CD8u chain, and (D) an endodomain comprising a signaling domain of a (C3 zeta chain, and, optionally, at least one other signaling domain. In one embodiment, the CAR further comprises a CD137 signaling domain and a CD3 zeta chain signaling domain. In some embodiments, the humanized antibodies or compositions are administered through an intravenous injection or through intra-peritoneal and subcutaneous methods. Such methods include administering a therapeutic agent to a subject in combination with an antibody or composition, such that the antibody targets delivery of the agent to an IL13Rα2-expressing cell. In another embodiment, the humanized antibodies or compositions are administered intra-tumorally to the site of the tumor.

In another embodiment, the disclosure provides a method for inducing lysis of a target cell, particularly a tumor cell, comprising contacting the target cell with a bispecific antibody described herein in the presence of a T cell, particularly a cytotoxic T cell. In some embodiments, the method is done in vivo in a subject having a tumor, particularly in some embodiments, glioblastoma.

### Definitions

Administering. As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent.

Antigen. The term “antigen,” as used herein, refers to any molecule that is recognized by the immune system and that can stimulate an immune response.

Chimeric antibody. The term “chimeric antibody” refers to an antibody comprising a variable region (i.e., binding region) from one source or species and at least a portion of a constant region derived from a different source or species. Other forms of “chimeric antibodies” are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques, which are now well known in the art. For example, chimeric antibodies are commonly isolated from a host cell (e.g., an SP2-0, NS0 or CHO cell) or from an animal (e.g., a mouse) that is transgenic for immunoglobulin genes or antibodies.

Complementarity determining region (CDR). The term “complementarity determining region” or “CDR,” as used herein, refers to part of the variable chains in immunoglobulins (antibodies) and T cell receptors where these molecules bind to their specific antigen. As the most variable parts of the molecules, CDRs are crucial to the diversity of antigen specificities generated by lymphocytes. There are three CDRs (CDR1, CDR2 and CDR3), arranged non-consecutively, on the amino acid sequence of a variable domain of an antigen receptor. Since the antigen receptors are typically composed of two variable domains (on two different polypeptide chains: the heavy and light chain), there are six CDRs for each antigen receptor that can collectively come into contact with the antigen. A single whole antibody molecule has two antigen receptors and therefore contains twelve CDRs. Sixty CDRs can be found on a pentameric IgM molecule. Within the variable domain, CDR1 and CDR2 may be found in the variable (V) region of a polypeptide chain, and CDR3 includes some of V, all of diversity (D, heavy chains only) and joining (J) regions. Since most sequence variation associated with immunoglobulins and T cell receptors is found in the CDRs, these regions are sometimes referred to as hypervariable regions. Among these, CDR3 shows the greatest variability as it is encoded by a recombination of VJ in the case of a light chain region and VDJ in the case of heavy chain regions.

Monoclonal antibody. The terms “monoclonal antibody” or “monoclonal antibody composition” as used herein refer to a preparation of antibody molecules of a single amino acid composition. Monoclonal antibodies also include “human monoclonal antibodies”, which display a single binding specificity and have variable and constant regions derived from human germline immunoglobulin sequences. Human monoclonal antibodies can be produced by a hybridoma, which includes a B cell obtained from a transgenic nonhuman animal (e.g., a transgenic mouse) having a genome comprising a human heavy chain transgene and a human light chain transgene fused to an immortalized cell.

Percentage of sequence similarity. “Percentage of sequence similarity” or “percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Karlin and Altschul, 1990, Proc. Natl. Acad. Sci. USA 87: 2267-2268; Altschul et al., 1997, Nucl. Acids Res. 25: 3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user. The term “substantial identity” of amino acid sequences for purposes of this invention normally means polypeptide sequence identity of at least 40%. Preferred percent identity of polypeptides can be any integer from 40% to 100%. More preferred embodiments include at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100%.

Protein. The terms “protein” and “polypeptide” are used interchangeably herein to designate a series of amino acid residues connected to by peptide bonds between the alpha-amino and carboxy groups of adjacent residues. The terms “protein” and “polypeptide” refer to a polymer of protein amino acids, including modified amino acids (e.g., phosphorylated, glycated, glycosylated, etc.) and amino acid analogs. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing. The antibodies of the present invention are polypeptides.

Nucleic Acid. The term “nucleic acid” as used herein includes “polynucleotide,” “oligonucleotide,” and “nucleic acid molecule,” and generally means a polymer of DNA or RNA, which may be single-stranded or double-stranded, synthesized or obtained (e.g., isolated and/or purified) from natural sources, which may contain natural, non-natural or altered nucleotides, and which may contain a natural, non-natural or altered internucleotide linkage, such as a phosphoroamnidate linkage or a phosphorothioate linkage, instead of the phosphodiester found between the nucleotides of an unmodified oligonucleotide.

Subject. As used herein, “subject” or “patient” refers to mammals and non-mammals. A “mammal” may be any member of the class Mammalia including, but not limited to, humans, non-human primates (e.g., chimpanzees, other apes, and monkey species), farm animals (e.g., cattle, horses, sheep, goats, and swine), domestic animals (e.g., rabbits, dogs, and cats), or laboratory animals including rodents (e.g., rats, mice, and guinea pigs). Examples of non-mammals include, but are not limited to, birds, and the like. The term “subject” does not denote a particular age or sex. In one specific embodiment, a subject is a mammal, preferably a human. In a preferred embodiment, the human has IL13Rα2 expressing tumor. In one example, the subject has glioblastoma.

Therapeutic agent. As used herein, the term “therapeutic agent” refers to any synthetic or naturally occurring biologically active compound or composition of matter which, when administered to a subject, induces a desired pharmacologic, immunogenic, and/or physiologic effect by local and/or systemic action. The term, therefore, encompasses those compounds or chemicals traditionally regarded as drugs, chemotherapeutics, and biopharmaceuticals including molecules such as proteins, peptides, hormones, nucleic acids, gene constructs and the like. Examples of therapeutic agents are described in well-known literature references, such as the Merck Index (14th edition), the Physicians' Desk Reference (64th edition), and The Pharmacological Basis of Therapeutics (12th edition), and they include, without limitation, substances used for the treatment, prevention, diagnosis, cure or mitigation of a disease or illness; substances that affect the structure or function of the body, or pro-drugs, which become biologically active or more active after they have been placed in a physiological environment.

Treating. As used herein, “treating” or “treatment” describes the management and care of a subject for the purpose of combating a disease, condition, or disorder. Treating includes the administration of an antibody or composition of present invention to prevent the onset of the symptoms or complications, to alleviate the symptoms or complications, or to eliminate the disease, condition, or disorder. Specifically, the antibodies or compositions disclosed herein can be used to treat a cancer that expresses IL13Rα2.

Therapeutically effective amount. The terms “effective amount” or “therapeutically effective amount” refer to an amount sufficient to effect beneficial or desirable biological or clinical results. That result can be reducing, alleviating, inhibiting or preventing one or more symptoms of a disease or condition, reducing, inhibiting or preventing the growth of cancer cells, reducing, inhibiting or preventing metastasis of the cancer cells or invasiveness of the cancer cells or metastasis, or reducing, alleviating, inhibiting or preventing one or more symptoms of the cancer or metastasis thereof, or any other desired alteration of a biological system. In some embodiments, the effective amount is an amount suitable to provide the desired effect, e.g., anti-tumor response. An anti-tumor response may be demonstrated, for example, by a decrease in tumor size or an increase in immune cell activation (e.g., CD8+ T cell activation).

Vector. The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”. Vectors comprise the nucleotide sequence encoding the antibodies described herein and a heterogeneous sequence necessary for proper propagation of the vector and expression of the encoded polypeptide. The heterogeneous sequence (i.e., sequence from a difference species than the polypeptide) can comprise a heterologous promoter or heterologous transcriptional regulatory region that allows for expression of the polypeptide. As used herein, the terms “heterologous promoter,” “promoter,” “promoter region,” or “promoter sequence” refer generally to transcriptional regulatory regions of a gene, which may be found at the 5′ or 3′ side of the polynucleotides described herein, or within the coding region of the polynucleotides, or within introns in the polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains (consensus sequences) responsible for the binding of RNA polymerase.

Expression vectors include all those known in the art including, without limitation, a yeast artificial chromosome, bacterial plasmid (e.g., naked or contained in liposomes), phagemid, shuttle vector, cosmid, virus (e.g., Sendai viruses, lentiviruses, retroviruses, adenoviruses, and adeno-associated viruses), chromosome, mitochondrial DNA, plastid DNA, and nucleic acid fragment. Generally, an expression vector comprises sufficient cis-acting elements for expression; other elements for expression can be supplied by the host cell or in an in vitro expression system.

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

The invention will be more fully understood upon consideration of the following non-limiting examples.

### EXAMPLES

### Example 1: Antibody Expression

The aim of this project is to humanize a mouse monoclonal antibody (mAb) using CDR grafting method without sacrificing the binding affinity of the parent antibody. The following experiments are presented in this Example:
1. Confirmation of antigen-antibody interaction
2. Humanized antibody design and construction
3. Selection of Humanized Antibodies

### Materials:

pCDNA3.4 expression vector and Expi293F cell (prepared by GenScript)
Biological safety cabinet (Thermo Scientific, Model. 1384)
Zhichu CO2 shaker incubator (Shanghai Zhichu Instrument, Model. ZCZY-BS8)
Expi293F medium (Gibco, Cat. No. A14351-01)
ExpiFectamine293 Transfection Kit (Gibco, Cat. No. A14525)
TPP Tubespin Bioreactor 50 (Cat. No. 87050)
125-ml shake flask (Corning, Cat. No. 431143)
500-ml shake flask (Corning, Cat. No. 431145)
Protein-A resin (GenScript, Cat. No. L0443)
Binding buffer: 0.15 M NaCl, 20 mM Nα2HPO4, pH 7.0
Elution buffer: 0.1 M Glycine-HCl, pH 2.5
Neutralization buffer: 1 M Tris-HCl, pH 9.0
Biacore T200/Biacore 8K (GE Healthcare)
Series S Sensor Chip Protein A (GE Healthcare, Cat. No.:29-1275-55)
HBS-EP: 10 mM HEPES, 150 mM NaCl, 3 mM EDTA, 0.005% Tween 20, pH 7.4
10 mM Glycine-HCl
Amino acid sequences of parental antibody
Human IL13Rα2

### Methods:

### Construction and Production of Chimeric Antibody

The DNA sequences encoding the chimeric antibody heavy and light chains were synthesized and inserted into the pCDNA3.4 vector to construct expression plasmids of full-length IgGs. Expression of chimeric antibody was conducted in Expi293F cell culture, and the supernatants were purified using an affinity purification column. The purified antibody was buffer-exchanged into PBS using dialysis bag. The concentration and purity of the purified protein was determined by OD280 and SDS-PAGE, respectively.

### Binding Confirmation of Chimeric Antibody

The affinity of chimeric antibody to the antigen human IL13Rα2 was determined using a surface plasmon resonance (SPR) biosensor, Biacore T200 (GE Healthcare). Antibody was immobilized on the sensor chip through Fc capture method. Human IL13Rα2 was used as the analyte with association time of 180 s and buffer flow was maintained for 600 s for dissociation. The data of dissociation (k_{d}) and association (kₐ) rate constants were obtained using Biacore T200 evaluation software. The equilibrium dissociation constants (K_{D}) were calculated from the ratio of k_{d} over kₐ.

### Humanization Design of Mouse Antibody

The humanized heavy chain and light chain were designed as described in design report. The designed plasmids of heavy chain and light chain were synthesized following GenScript's standard operating procedures (SOP).

### Production and Affinity Ranking of Humanized Antibodies

The designed plasmids of heavy chain and light chain were sent for 5 mL transfection following GenScript's standard operating procedures (SOP). For affinity ranking, antibodies were immobilized on the sensor chip through Fc capture method. Human IL13Rα2 was used as the analyte. The surface was regenerated before the injection of another antibody. The process was repeated until all antibodies are analyzed. The off-rates of antibodies were obtained from fitting the experimental data locally to 1:1 interaction model using the Biacore 8K evaluation software. The antibodies were ranked by their dissociation rate constants (off-rates, k_{d}). Based on the ranked results, the top three clones were selected.

### Construction and Production of Humanized IgGs

The top three clones were selected and expressed in Expi293F cell culture. The recombinant IgGs secreted to the medium were purified using resin A affinity chromatography following GenScript's SOP.

### Affinity Measurement of Purified Humanized IgGs

The affinity of purified antibodies binding to human IL13Rα2 was individually determined using Biacore T200. Antibodies were immobilized on the sensor chip through Fc capture method. Human IL13Rα2was used as the analyte. The data of dissociation (k_{d}) and association (kₐ) rate constants were obtained using Biacore T200 evaluation software. The equilibrium dissociation constants (K_{D}) were calculated from the ratio of k_{d} over kₐ.

### Results:

### Chimeric Antibody Production

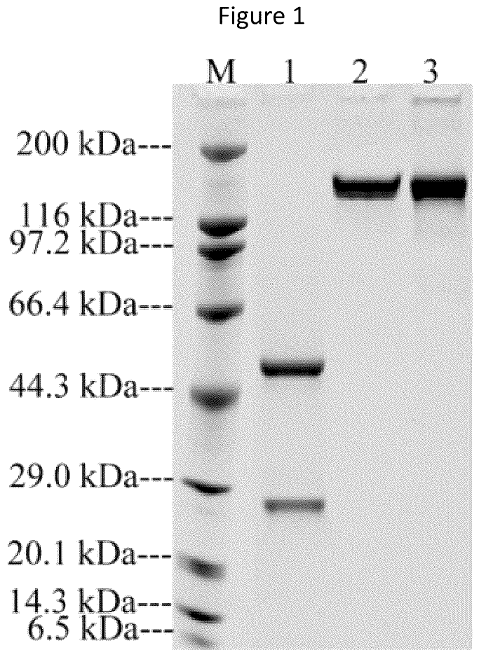

Chimeric antibody was expressed and purified according to GenScript's SOP, respectively. The purified IgG migrated as ~150 kDa band in SDS-PAGE under non-reducing conditions. Based on the SDS-PAGE result, the purity of IgG is >95% (FIG. 1). The yield of purified IgG from 100 ml cell culture was ~3.28 mg.

### Binding Confirmation of Chimeric Antibody by SPR

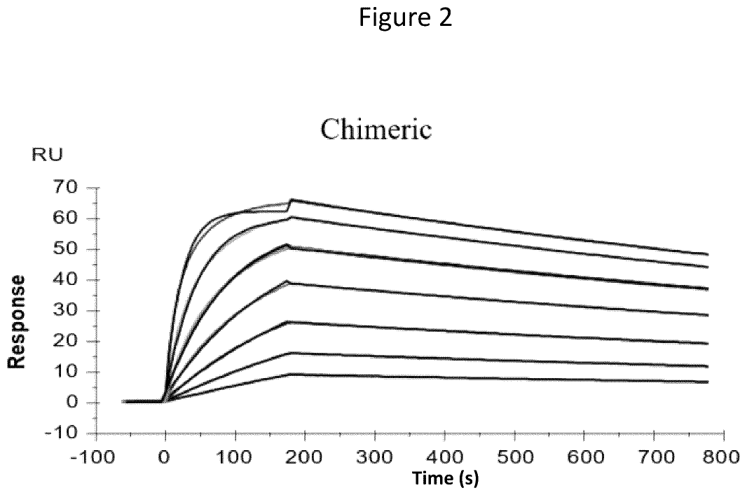

The results indicate that chimeric antibody can bind to the Human IL13Rα2. The affinity and kinetics of human IL13Rα2 binding to Chimeric IgG are summarized in Table 1 and the sensor-grams are shown in FIG. 2.

TABLE 1
The affinity and kinetics of human IL13Rα2 binding to chimeric IgG.

| Ligand | Analyte | kₐ (1/Ms) | k_{d} (1/s) | K_{D} (M) | Rmax | Chi²(RU²) |
|---|---|---|---|---|---|---|
| Chimeric | Human IL13Rα2 | 3.63E+05 | 5.23E−04 | 1.44E−09 | 66.51 | 0.196 |

### Affinity Ranking of Humanized Antibodies

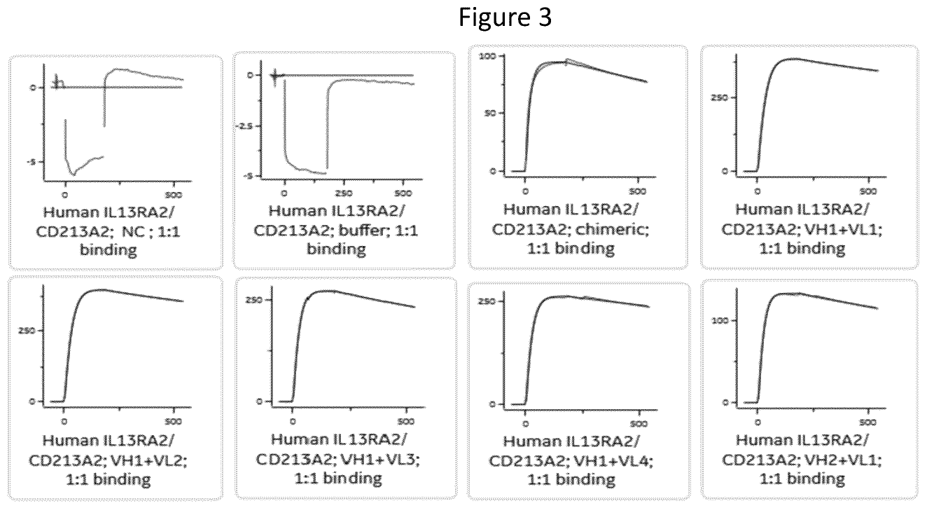

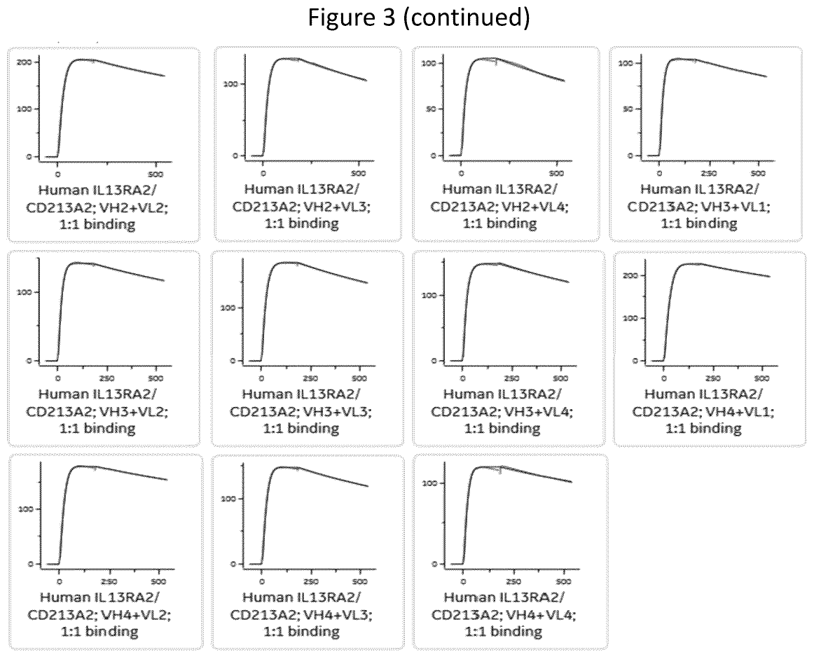

The affinity of human IL13Rα2 binding to humanized antibodies supernatant is summarized in Table 2 and the sensor-grams are shown in FIG. 3.

TABLE 2
The affinity and kinetics of human IL13Rα2 binding to humanized antibodies.

| | | | | | Rmax | |
|---|---|---|---|---|---|---|
| Ligand | Analyte | kₐ (1/Ms) | k_{d} (1/s) | K_{D} (M) | (RU) | Chi²(RU²) |
| NC | Human IL13Rα2 | NA | NA | NA | NA | NA |
| buffer | Human IL13Rα2 | NA | NA | NA | NA | NA |
| chimeric | Human IL13Rα2 | 5.11E+05 | 5.44E−04 | 1.07E−09 | 96 | 3.01E+00 |
| VH1 + VL1 | Human IL13Rα2 | 4.29E+05 | 3.29E−04 | 7.68E−10 | 388.7 | 1.38E+00 |
| VH1 + VL2 | Human IL13Rα2 | 4.12E+05 | 3.12E−04 | 7.58E−10 | 402.2 | 1.06E+00 |
| VH1 + VL3 | Human IL13Rα2 | 5.14E+05 | 4.63E−04 | 9.00E−10 | 275.8 | 9.21E−01 |
| VH1 + VL4 | Human IL13Rα2 | 4.49E+05 | 2.69E−04 | 5.99E−10 | 265.1 | 4.47E+00 |
| VH2 + VL1 | Human IL13Rα2 | 5.93E+05 | 4.17E−04 | 7.04E−10 | 134.8 | 1.03E+00 |
| VH2 + VL2 | Human IL13Rα2 | 6.48E+05 | 5.55E−04 | 8.57E−10 | 206.7 | 8.15E−01 |
| VH2 + VL3 | Human IL13Rα2 | 6.49E+05 | 7.61E−04 | 1.17E−09 | 137 | 1.07E+00 |
| VH2 + VL4 | Human IL13Rα2 | 5.55E+05 | 7.48E−04 | 1.35E−09 | 106.8 | 1.90E+00 |
| VH3 + VL1 | Human IL13Rα2 | 9.01E+05 | 5.95E−04 | 6.61E−10 | 104.7 | 3.10E−01 |
| VH3 + VL2 | Human IL13Rα2 | 8.52E+05 | 5.95E−04 | 6.98E−10 | 143.2 | 4.75E−01 |
| VH3 + VL3 | Human IL13Rα2 | 6.20E+05 | 7.02E−04 | 1.13E−09 | 189.9 | 7.11E−01 |
| VH3 + VL4 | Human IL13Rα2 | 6.25E+05 | 6.15E−04 | 9.84E−10 | 150.1 | 1.42E+00 |
| VH4 + VL1 | Human IL13Rα2 | 5.83E+05 | 4.24E−04 | 7.27E−10 | 229.1 | 1.04E+00 |
| VH4 + VL2 | Human IL13Rα2 | 7.46E+05 | 4.45E−04 | 5.97E−10 | 181.6 | 7.56E−01 |
| VH4 + VL3 | Human IL13Rα2 | 7.69E+05 | 6.75E−04 | 8.77E−10 | 149.3 | 6.47E−01 |
| VH4 + VL4 | Human IL13Rα2 | 8.44E+05 | 5.01E−04 | 5.94E−10 | 121 | 2.12E+00 |

### Production of Humanized IgGs

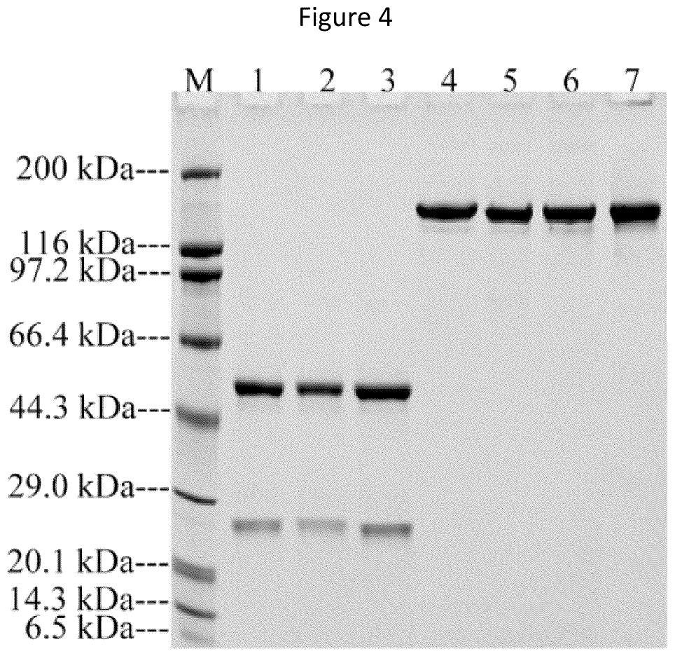

The top three humanized antibodies with the highest affinity for human IL13Rα2 were expressed and purified according to GenScript's SOP. The purified IgGs migrated as ~150 kDa band in SDS-PAGE under non-reducing condition, ~50 kDa and ~25 kDa bands under reducing condition. Judging from the SDS-PAGE, the purity of humanized IgGs were all over 95% (FIG. 4).

Affinity Measurement of Purified hIgGs

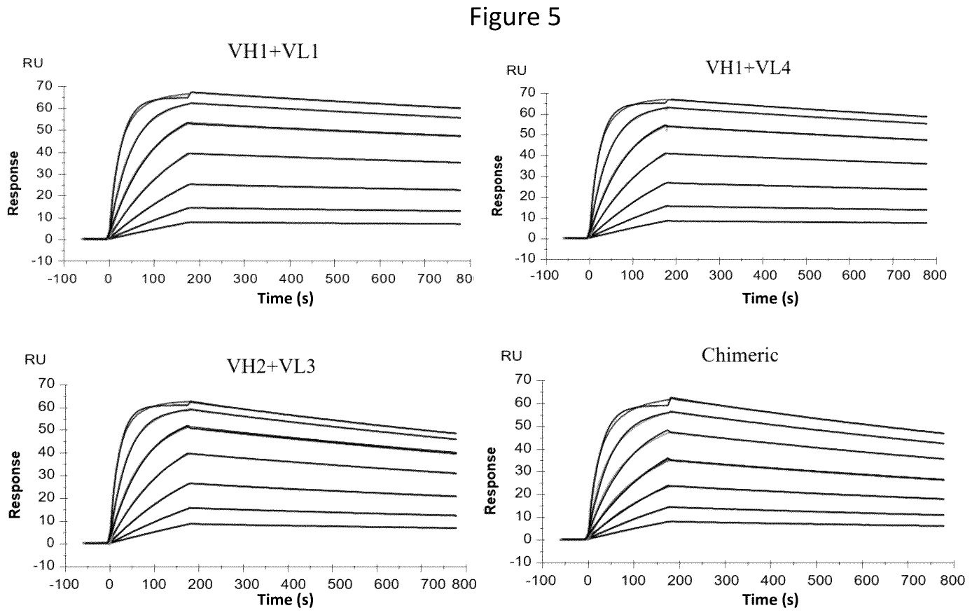

The affinity of human IL13Rα2 binding to antibodies is summarized in Table 3 and the sensor-grams are shown in FIG. 5.

TABLE 3
The affinity of human IL13Rα2 binding to selected humanization hIgGs.

| | | | | | Rmax | |
|---|---|---|---|---|---|---|
| Ligand | Analyte | kₐ (1/Ms) | k_{d} (1/s) | K_{D} (M) | (RU) | Chi²(RU²) |
| VH1 + VL1 | Human IL13Rα2 | 6.76E+05 | 1.91E−04 | 2.83E−10 | 67.57 | 0.0784 |
| VH1 + VL4 | Human IL13Rα2 | 7.41E+05 | 2.20E−04 | 2.97E−10 | 67.13 | 0.0821 |
| VH2 + VL3 | Human IL13Rα2 | 8.20E+05 | 4.20E−04 | 5.13E−10 | 62.6 | 0.0735 |
| Chimeric | Human IL13Rα2 | 3.30E+05 | 4.77E−04 | 1.45E−09 | 62.68 | 0.169 |

### SUMMARY

Mouse monoclonal antibody (mAb) was successfully humanized. Four humanized heavy chains and four humanized light chains were designed, synthesized, and individually inserted into an expression vector. The humanized antibodies were expressed and used for affinity ranking test. Finally, three humanized antibodies with a similar binding affinity as the chimeric antibody were identified and purified.

### Example 2: Affinity of Antibodies

In the following Example, the affinity of human IL13Rα2 binding to selected antibodies, including antibodies in which the VH region contained a D55E or G56A point mutation, was measured using Biacore T200 (GE Healthcare) using the parameters shown in Table 4.

TABLE 4
Binding parameters.
Immobilization

| | |
|---|---|
| Capture time(s) | 25 |
| Flow rate(μl/min) | 10 |

Association & Dissociation

| | |
|---|---|
| Association contact time(s) | 180 |
| Dissociation contact time(s) | 600 |
| Flow rate(μl/min) | 30 |
| Sample concentrations(nM) | 0.9375, 1.875, 3.75, 7.5, 15, 30, 60 |

Surface regeneration

| | |
|---|---|
| Regeneration buffer | 10 mM Glycine-HCl |
| Contact time(s) | 30 |
| Flow rate(μl/min) | 30 |

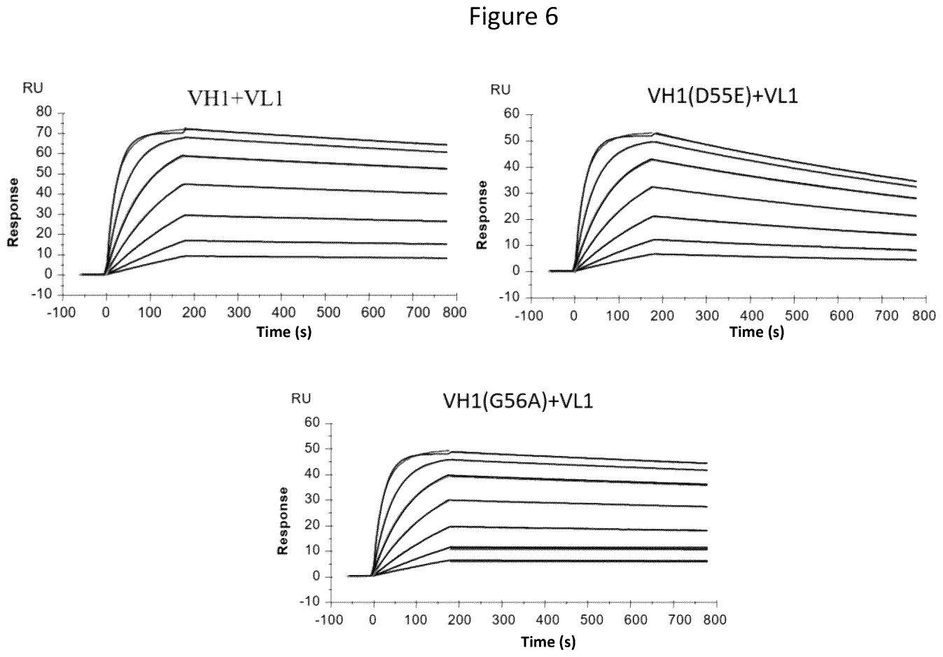

The affinity and kinetics of human L13Rα2 binding to selected antibodies are summarized in Table 5, and the sensor-grams were shown in FIG. 6.

TABLE 5
The affinity and kinetics of L13Rα2 binding to selected ligands.

| Ligand | kₐ (1/Ms) | k_{d} (1/s) | K_{D} (M) | Rmax | Chi² (RU²) |
|---|---|---|---|---|---|
| VH1 + VL1 | 7.40E+05 | 1.90E−04 | 2.57E−10 | 72.19 | 0.0771 |
| VH1(D55E) + | 7.38E+05 | 7.12E−04 | 9.65E−10 | 53.44 | 0.0384 |
| VL1 | | | | | |
| VH1(G56A) + | 7.27E+05 | 1.56E−04 | 2.15E−10 | 48.67 | 0.043 |
| VL1 | | | | | |

### Conclusions:

The D55E mutation reduced the affinity of the humanized anti-IL13Rα2 antibody, while the G56A did not. Nevertheless, the humanized anti-IL13Rα2 antibody VH1 (D55E)+VL 1 still exceeded the affinity of the chimeric antibody (i.e, the murine VH and VL sequences with human constant region).

### Example 3: Post-translational Modifications Analysis

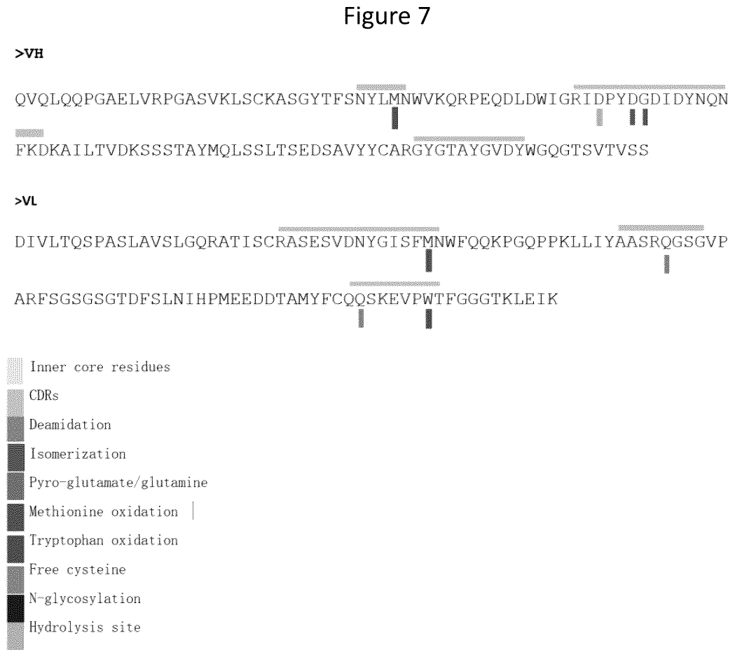

The grafted sequence was inspected for potential liability like N-glycosylation sites, post-translational modifications, and unpaired cysteine residues, which may affect the binding activity of the grafted antibody. Residues at risk for post-translational modification (PTM) are outlined in Table 6. The result of the PTM analysis performed on the mouse monoclonal antibody (mAb) sequence is shown in FIG. 7.

TABLE 6
Residues at risk for post-translational modification.

| Post-translational modifications | Sequence or residues |
|---|---|
| N-glycosylation | Asn-x-Ser/Thr (where x is |
| | not Pro) |
| Aspartate isomerization to | Asp-Gly, Asp-Ser |
| isoaspartate | |
| Deamidation of asparagine and | Asn-Gly, Asn-Ser are most |
| glutamine to isoaspartate and | prominent; Gln-Gly, Gln-Ser |
| isoglutamate, respectively | |
| Methionine oxidation | Surface-exposed Met residues; |
| | particular issue if Met in CDRs |
| Tryptophan oxidation | Surface-exposed Trp residues; |
| | particular issue if Trp in CDRs |
| Pyroglutamate formation from | N-terminal Glu or Gln residue |
| N-terminal glutamate or | |
| glutamine | |
| Free -thiol group | No pairing cysteine residue |
| Hydrolysis | Asp-Pro |

### Example 4: Stability of Antibodies

In the following example, the inventor analyzed the stability of several forms of a humanized anti-IL13Rα2 antibody described herein. These forms include a mutant that comprises a D55E mutation in VH domain (“VH1D55E-VL 1”), a mutant that comprises a G56A mutation in VH domain (“VH1G56A-VL 1”), and the un-mutated form of the humanized anti-IL13Rα2 antibody (“VH1-VL 1”). The stability of these humanized antibodies is compared to that of the parental chimeric murine antibody (“Ab”; the VH and VL of murine Ab clone 47 in human constant regions). All antibodies comprised the same constant regions.

### Materials:

TABLE 7
Sample information.

| | Amount | | Buffer | | Theoretical |
|---|---|---|---|---|---|
| Antibody | & | Concen- | component | Isoelectric | extinction |
| name | volume | tration | and pH | point | coefficient |
| Chimeric | 26 mg, | 2.1 mg/mL | PBS, pH | 6.91 | 1.433 |
| Ab | 12 mL | | 7.4 | | |
| Humanized | 31 mg, | 3.1 mg/mL | PBS, pH | 7.20 | 1.475 |
| VH1-VL1 | 10 mL | | 7.4 | | |
| Humanized | 31 mg, | 2.8 mg/mL | PBS, pH | 7.20 | 1.475 |
| VH1D55E- | 11 mL | | 7.4 | | |
| VL1 | | | | | |
| Humanized | 30 mg, | 3.1 mg/mL | PBS, pH | 7.20 | 1.475 |
| VHG56A- | 9.5 mL | | 7.4 | | |
| VL1 | | | | | |

Sample Handling. For conditions of low pH 3.5, the above samples went through the buffer exchange, and then the aliquots were made. For condition of 40° C., the aliquots were made directly without buffer exchange.

TABLE 8
Sample handling conditions.

| | Starting | Investigation | Time points |
|---|---|---|---|
| Conditions | buffer | conditions | (Test items) |
| D 0 | PBS, pH 7.4 | D 0 | A, B, C |
| Low pH 3.5 | PBS, pH 7.4 | Low pH 2 h, 4 h | B, C |
| 40° C. | PBS, pH 7.4 | D 7, D 14, D 28 | B, C |

A: DLS (Tagg/onset);
B: UV280;
C: SEC-HPLC;
D 0: Day 0;
D 7: Day 7;
D 14: Day 14;
D 28: Day 28;

### Results:

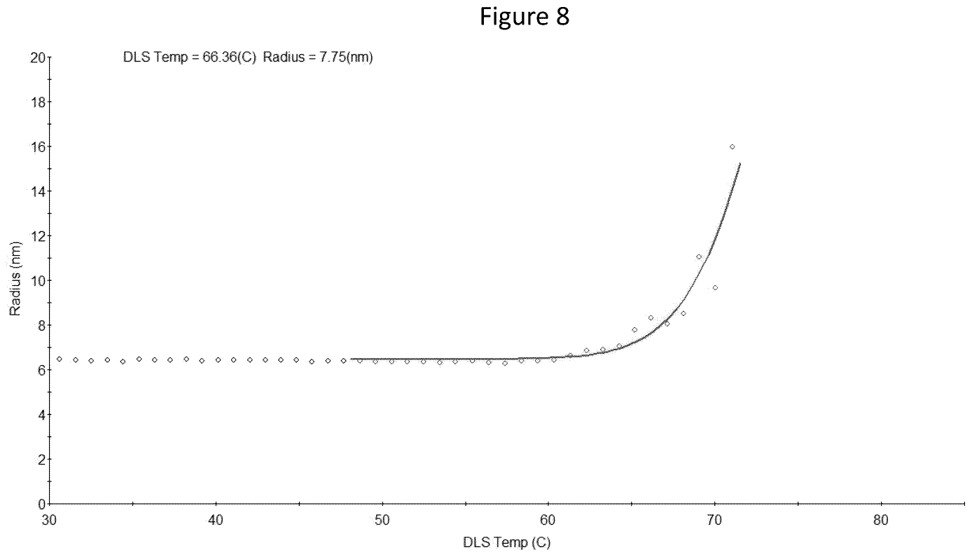

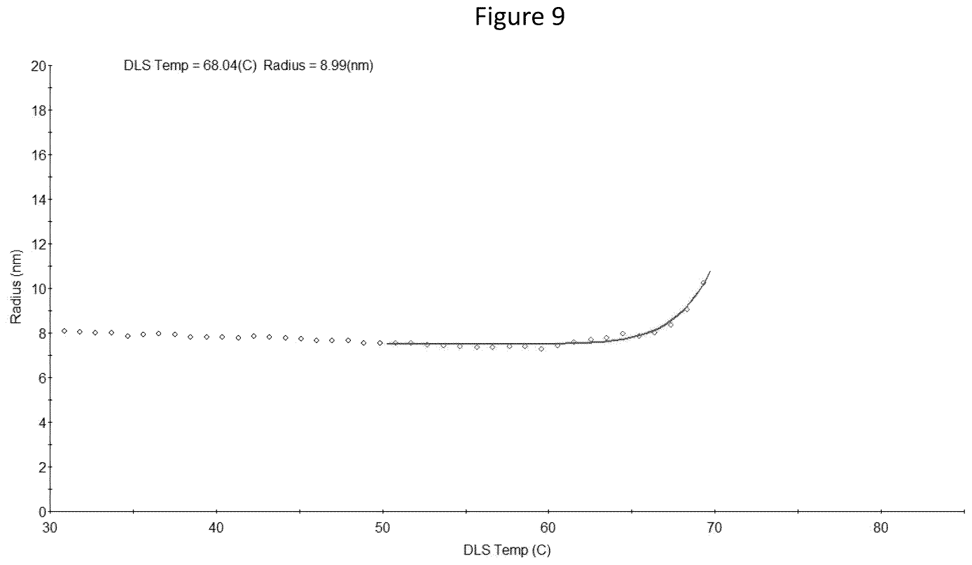

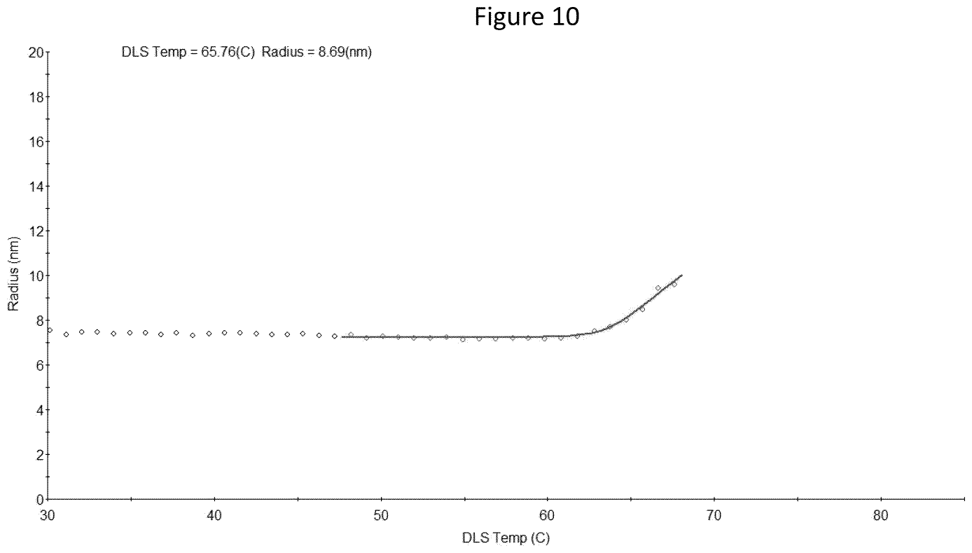

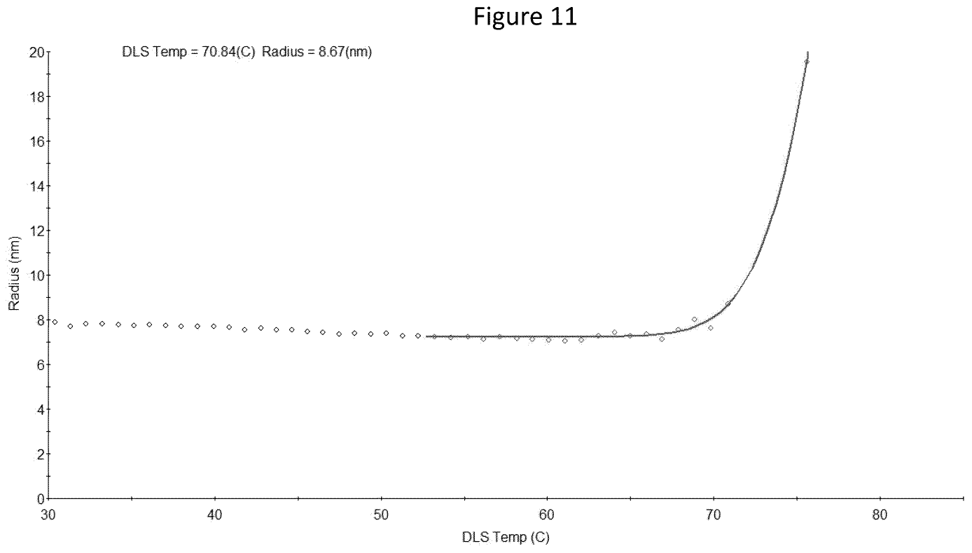

DLS. Dynamic light scattering (DLS) measures the Tonset/agg (onset temperature at which aggregates are first detected. The DLS results are presented in Table 9, and the corresponding thermograms are presented in FIG. 8-11. As seen in Table 9, all humanized antibodies made surprisingly had increased stability over the murine chimeric antibody (“Ab”). However, the G56A mutation increased the stability and melting temperature the most.

TABLE 9
DLS test results.

| Sample name | Tonset/agg |
|---|---|
| Ab-D 0 | 66.36° C. |
| VH1-VL1-D 0 | 68.04° C. |
| VH1D55E-VL1-D 0 | 65.76° C. |
| VHG56A-VL1-D 0 | 70.84° C. |

UV280. The humanized antibodies are stable and do not result in protein loss, even when incubated at high temperature or low pH for extended periods of time as determined by UV280. The UV280 results are presented in Table 10.

TABLE 10
UV280 test results.

| Sample name/ | | | | Average | CV | | Conc. |
|---|---|---|---|---|---|---|---|
| conditions | OD value | | | OD value | % | E | (mg/mL) |
| Ab-D0 | 0.54978 | 0.54869 | 0.54883 | 0.54910 | 0.088 | 1.449 | 1.14 |
| Ab-40C-D7 | 0.25935 | 0.25903 | 0.25856 | 0.25898 | 0.125 | 1.449 | 1.07 |
| Ab-40C-D14 | 0.27521 | 0.27538 | 0.27506 | 0.27522 | 0.047 | 1.449 | 1.14 |
| Ab-40C-D28 | 0.27557 | 0.27483 | 0.27497 | 0.27512 | 0.117 | 1.449 | 1.14 |
| Ab-LowpH-2h | 0.60794 | 0.60649 | 0.60664 | 0.60702 | 0.107 | 1.449 | 3.77 |
| Ab-LowpH-4h | 0.63343 | 0.63352 | 0.63272 | 0.63322 | 0.057 | 1.449 | 3.50 |
| VH1-VL1-D0 | 0.31577 | 0.31561 | 0.31475 | 0.31538 | 0.142 | 1.461 | 1.94 |
| VH1-VL1-40C-D7 | 0.32780 | 0.32722 | 0.32704 | 0.32735 | 0.099 | 1.461 | 2.02 |
| VH1-VL1-40C-D14 | 0.31151 | 0.30987 | 0.31156 | 0.31098 | 0.252 | 1.461 | 1.92 |
| VH1-VL1-40C-D28 | 0.33300 | 0.33297 | 0.33279 | 0.33292 | 0.028 | 1.461 | 2.05 |
| VH1-VL1-LowpH-2h | 0.47977 | 0.48082 | 0.47969 | 0.48009 | 0.107 | 1.461 | 10.19 |
| VH1-VL1-LowpH-4h | 0.51217 | 0.50994 | 0.51015 | 0.51075 | 0.197 | 1.461 | 8.39 |
| VH1D55E-VL1-D0 | 0.32923 | 0.32900 | 0.32813 | 0.32879 | 0.144 | 1.461 | 1.80 |
| VHID55E-VL1-40C-D7 | 0.33728 | 0.33636 | 0.33659 | 0.33674 | 0.116 | 1.461 | 1.84 |
| VHID55E-VL1-40C-D14 | 0.32553 | 0.32506 | 0.32465 | 0.32508 | 0.111 | 1.461 | 1.78 |
| VHID55E-VL1-40C-D28 | 0.34463 | 0.34495 | 0.34321 | 0.34426 | 0.220 | 1.461 | 1.89 |
| VH1D55E-VL1-LowpH- | 0.47308 | 0.47094 | 0.47071 | 0.47158 | 0.226 | 1.461 | 10.97 |
| 2h | | | | | | | |
| VH1D55E-VL1-LowpH- | 0.48076 | 0.48201 | 0.48118 | 0.48132 | 0.108 | 1.461 | 9.88 |
| 4h | | | | | | | |
| VHG56A-VL1-D0 | 0.29295 | 0.29304 | 0.29348 | 0.29316 | 0.079 | 1.461 | 1.81 |
| VHG56A-VL1-40C-D7 | 0.31669 | 0.31506 | 0.31507 | 0.31561 | 0.243 | 1.461 | 1.94 |
| VHG56A-VL1-40C-D14 | 0.30588 | 0.30512 | 0.30516 | 0.30539 | 0.114 | 1.461 | 1.88 |
| VHG56A-VL1-40C-D28 | 0.31476 | 0.31519 | 0.31535 | 0.31510 | 0.079 | 1.461 | 1.94 |
| VHG56A-VL1-LowpH- | 0.46417 | 0.46515 | 0.4657 | 0.46501 | 0.136 | 1.461 | 6.68 |
| 2h | | | | | | | |
| VHG56A-VL1-LowpH- | 0.48869 | 0.48798 | 0.49163 | 0.48943 | 0.323 | 1.461 | 7.04 |
| 4h | | | | | | | |

Note:
VHG56A-VL1-40C-D7 indicates that VHG56A-VL1 treated at 40° C. for 7 days; VHG56A-VL1-LowpH-4h indicates VHG56A-VL1 treated under low pH conditions for 4 hours; the same principle is made for othersample names.

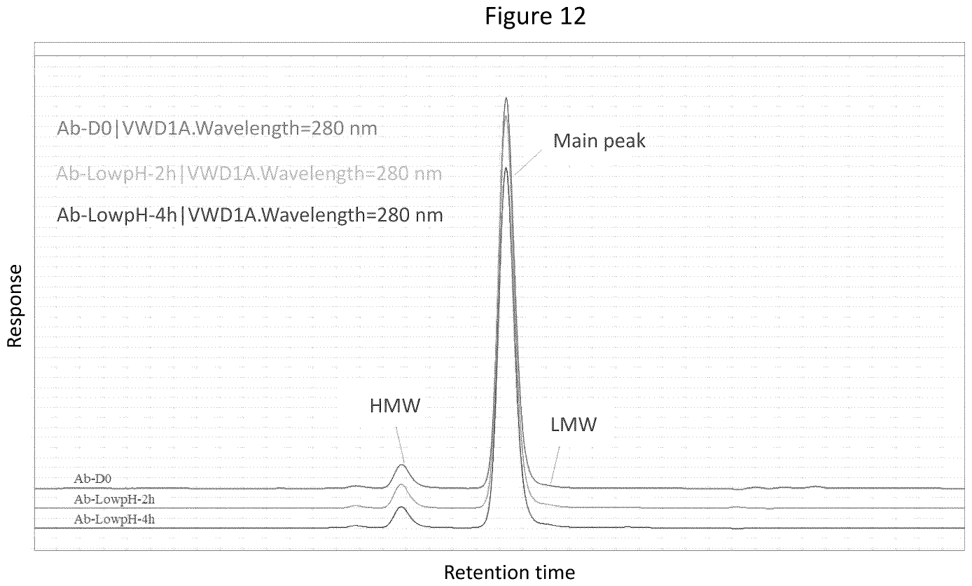

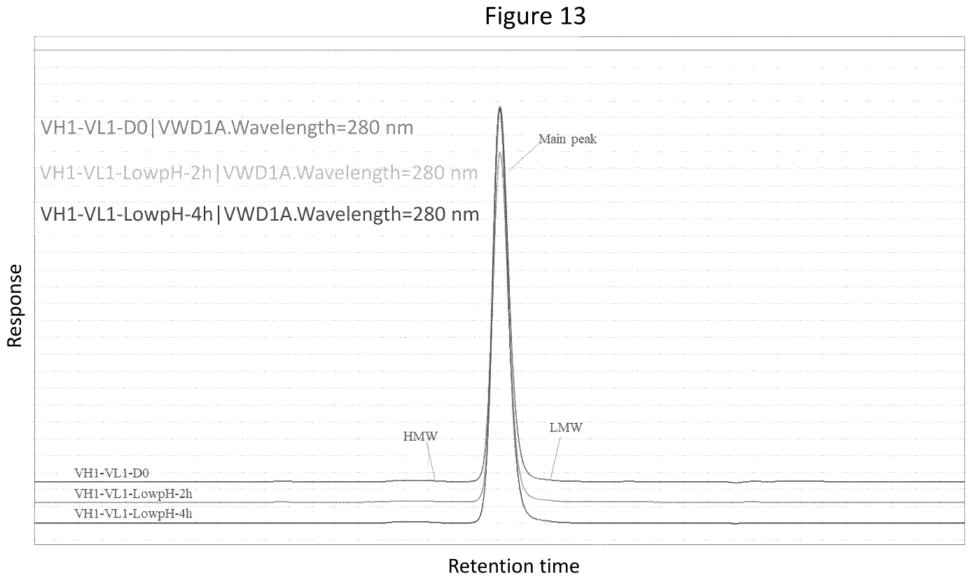

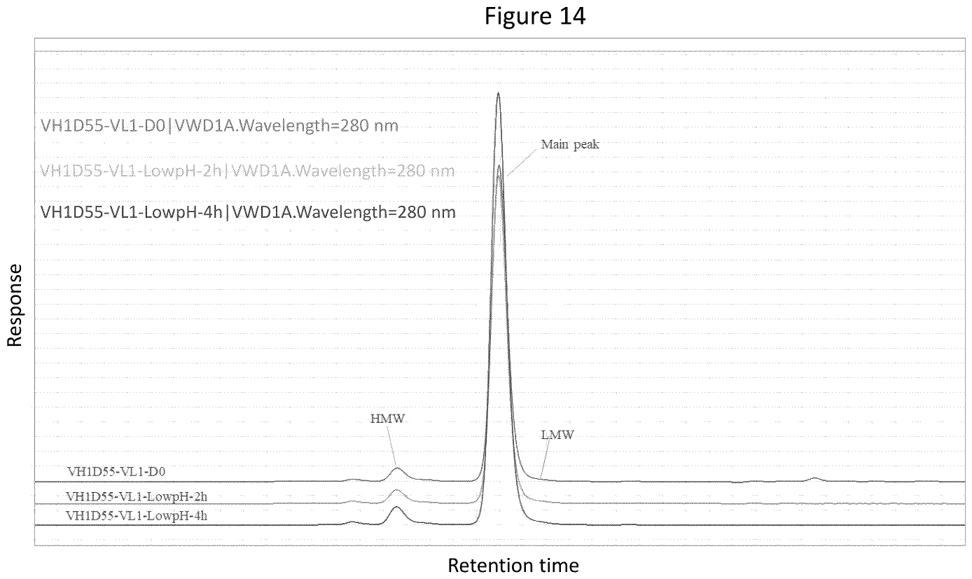

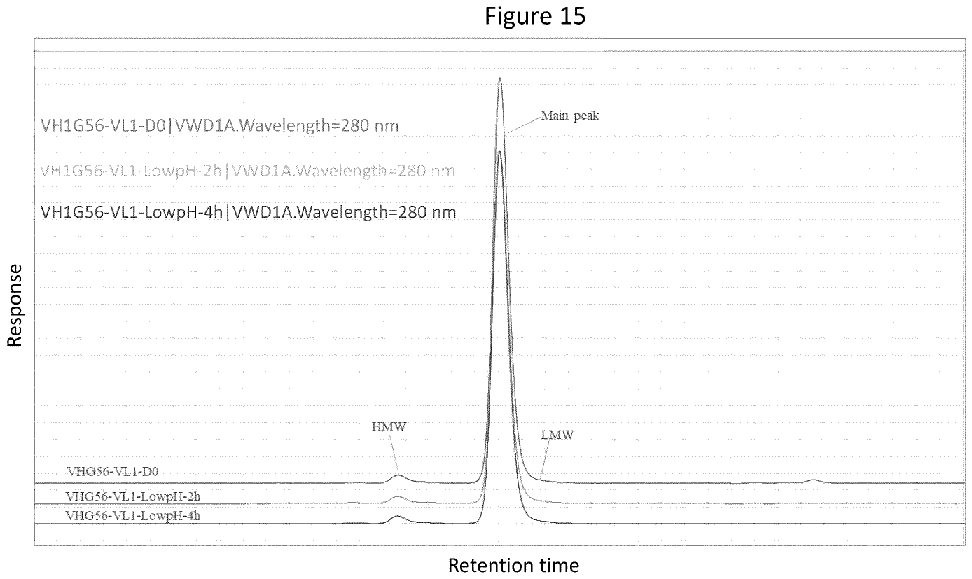

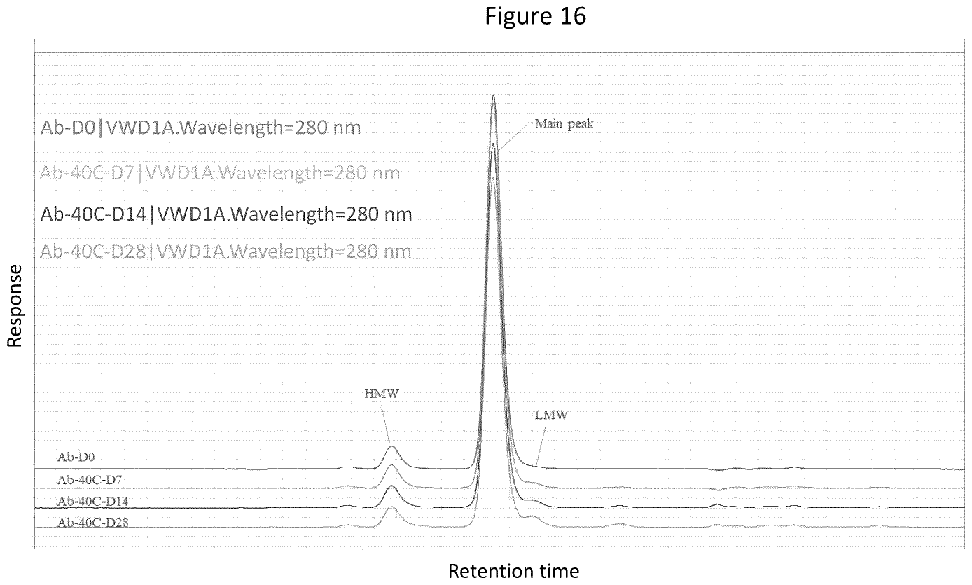

pH 3.5 stability of SEC-HPLC measurement. The pH 3.5 stability ofsize-exclusion chromatography (SEC)-HPLC measurement is summarized in the following table, where samples of DO is served as starting point, respectively. The humanized antibodies had high purity determined by SEC-HPLC for each molecule. The pH 3.5 stability of SEC-HPLC measurements are presented in Table 11, and the corresponding SEC-HPLC chromatograms are presented in FIG. 12-15.

TABLE 11
pH 3.5 stability of SEC-HPLC test results.

| Sample | | HMW | Main | LMW | |
|---|---|---|---|---|---|
| name | Conditions | (%) | peak (%) | (%) | RT(min) |
| Ab | D 0 | 6.77 | 92.33 | 0.91 | 12.628 |
| | LowpH-2 h | 6.51 | 91.95 | 1.54 | 12.622 |
| | LowpH-4 h | 6.28 | 92.46 | 1.26 | 12.622 |
| VH1-VL1 | D 0 | 1.26 | 98.56 | 0.18 | 12.525 |
| | LowpH-2 h | 0.90 | 98.97 | 0.13 | 12.528 |
| | LowpH-4 h | 0.91 | 98.97 | 0.12 | 12.520 |
| VH1D55E- | D 0 | 6.07 | 93.84 | 0.09 | 12.467 |
| VL1 | LowpH-2 h | 5.42 | 94.47 | 0.11 | 12.457 |
| | LowpH-4 h | 5.44 | 94.46 | 0.09 | 12.451 |
| VHG56A- | D 0 | 2.91 | 96.83 | 0.27 | 12.518 |
| VL1 | LowpH-2 h | 2.69 | 97.18 | 0.13 | 12.513 |
| | LowpH-4 h | 2.78 | 97.13 | 0.10 | 12.508 |

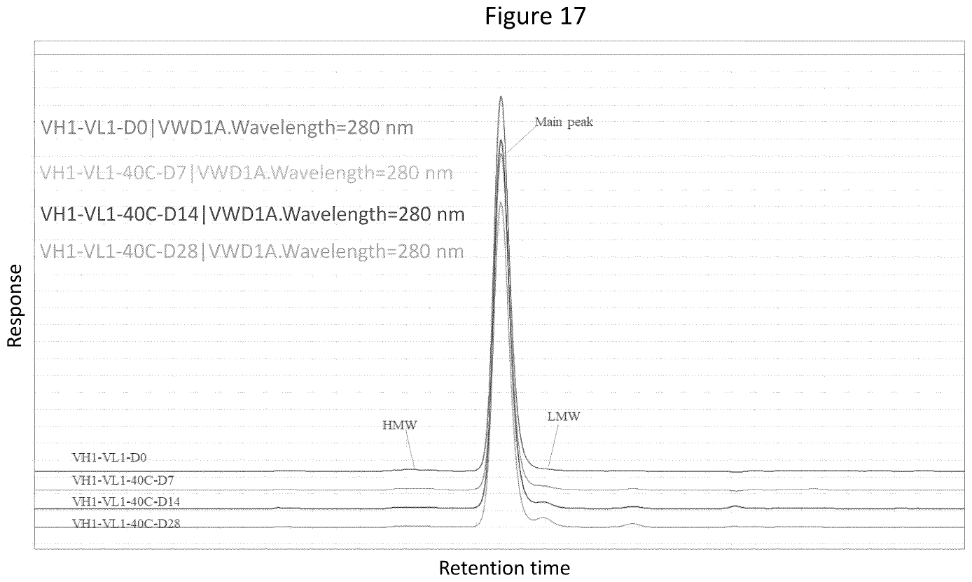

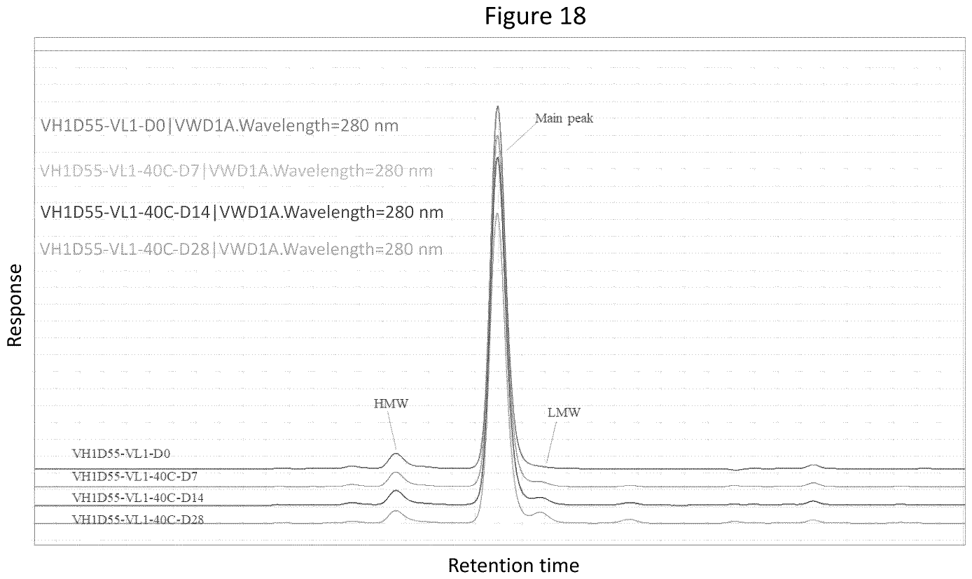

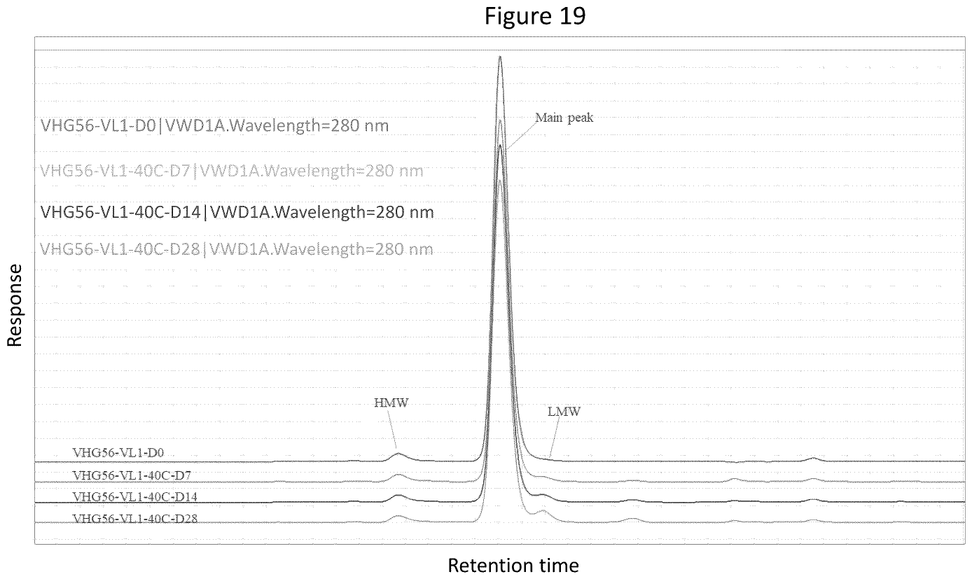

40° C. stability of SEC-HPLC measurement. The 40° C. stability of SEC-HPLC measurement is summarized in Table 12, and the corresponding SEC-HPLC chromatograms are presented in FIG. 16-19. Sample DO serves as a starting reference point. Certain changes were observed in terms of purity determined by SEC-HPLC for each molecule. These changes follow a similar trend: the main peak percentage decreased, low molecular weight species (LMW) increased, and high molecular weight species (HMW) had no obvious change. No obvious difference in change rate was observed among the four molecules. All humanized antibodies had less low molecular weight products (i.e., had less degradation products), and had less high molecular weight products (i.e., less aggregation).

TABLE 12
40° C. stability of SEC-HPLC test results.
40° C. stability of SEC-HPLC test results

| Sample | | HMW | Main | LMW | |
|---|---|---|---|---|---|
| name | Conditions | (%) | peak (%) | (%) | RT(min) |
| Ab | D 0 | 6.77 | 92.33 | 0.91 | 12.628 |
| | 40 C.-D 7 | 6.78 | 91.50 | 1.72 | 12.624 |
| | 40 C.-D 14 | 6.66 | 91.15 | 2.20 | 12.620 |
| | 40 C.-D 28 | 6.72 | 89.19 | 4.09 | 12.616 |
| VH1-VL1 | D 0 | 1.26 | 98.56 | 0.18 | 12.525 |
| | 40 C.-D 7 | 1.38 | 97.22 | 1.41 | 12.527 |
| | 40 C.-D 14 | 1.37 | 96.23 | 2.41 | 12.525 |
| | 40 C.-D 28 | 1.44 | 94.28 | 4.29 | 12.521 |
| VH1D55E- | D 0 | 6.07 | 93.84 | 0.09 | 12.467 |
| VL1 | 40 C.-D 7 | 5.80 | 92.49 | 1.71 | 12.464 |
| | 40 C.-D 14 | 5.87 | 91.00 | 3.13 | 12.464 |
| | 40 C.-D 28 | 5.85 | 89.10 | 5.06 | 12.458 |
| VHG56A- | D 0 | 2.91 | 96.83 | 0.27 | 12.518 |
| VL1 | 40 C.-D 7 | 3.00 | 95.34 | 1.66 | 12.518 |
| | 40 C.-D 14 | 3.08 | 94.02 | 2.90 | 12.515 |
| | 40 C.-D 28 | 3.14 | 91.95 | 4.93 | 12.512 |

### Conclusions:

Based on the DLS tests, the Tonset/agg of Ab, VH1-VL 1, VH1D55E-VL 1, and VHG56A-VL 1 are 66.36° C., 68.04° C., 65.76° C., and 70.84° C., respectively. These results demonstrate that the both VH1-VL 1 and VHG56A-VL 1 have higher melting temperatures than the parental chimeric antibody (“Ab”; the VH and VL of murine Ab clone 47 in human constant regions).
Based on the initial purity (DO) determination by SEC-HPLC, the main peak percentage of VH1-VL 1 is the highest, followed by VHG56A-VL 1, then VH1D55E-VL 1, and Ab is the lowest. Thus, all humanized antibodies had higher purity and less degradation products and aggregated products than the parental chimeric antibody.
Based on the low pH 3.5 stability test, no obvious changes in purity were observed for each molecule. Based on the 40° C. stability test, certain changes were observed in the purity of each molecule. These changes followed a similar trend: the main peak percentage decreased, LMW increased, and HMW had no obvious change. The results demonstrate that all four forms of the antibody are relatively stable at low pH and high temperature. VH1-VL 1 and VH1G56A-VL 1 showed less HIW (%) than the chimeric antibody (Ab) and humanized VH1D55E-VL 1.

### Example 5: Affinity of Antibodies

In the following Example, the inventor measured the binding affinity of two mutant forms of a humanized anti-IL13Rα2 antibody described herein to human IL13Rα2 using a Biacore T200. One of these mutants comprises a single W100F mutation in VL domain (“Ab Vh1-VL 1W100F”). The second mutant comprises the same W100F mutation in VL domain as well as a G56A mutation in the VH domain (“Ab Vh1G56A-VL 1W100F”).

### Materials:

TABLE 13
Samples

| Samples | MW(KDa) | Concentration(mg/ml) |
|---|---|---|
| Ab Vh1-VL1W100F | 150 | 0.706 |
| Ab Vh1G56A/VL1W100F | 150 | 0.734 |
| Human IL13Ra2 | 44.18 | 0.5 |

TABLE 14
Instrument and reagents

| Names | Cat. No. | Lot. No. | Vendor |
|---|---|---|---|
| Biacore T200: | N/A | N/A | GE Healthcare |
| GR18010468 | | | |
| HBS-EP + buffer | BR-1006-69 | 31644 | GE Healthcare |
| Series S Sensor Chip | 29-1275-55 | 10299112 | GE Healthcare |
| Protein A | | | |
| Regeneration buffer: 10 | BR-1003-54 | 01/05/2021 | Genscript |
| mM Glycine-HCl pH 1.7 | | | |

### Methods:

The assay was performed at 25° C. and the running buffer was HBS-EP+. Diluted antibodies were captured on the sensor chip through Fe capture method. Human L13Rα2 was used as the analyte. Running buffer was injected as the dissociation phase. The running configuration is detailed in Table 15 below.

TABLE 15
Running configuration
Capture

| Ligand | antibodies |
|---|---|
| Capture time(s) | 15 |

Association & Dissociation

| | |
|---|---|
| Association contact time(s) | 180 |
| Dissociation contact time(s) | 600 |
| Flow rate(μl/min) | 30 |
| Sample concentrations(nM) | 80, 40, 20, 10, 5, 2.5, 1.25, 0.625 |

Surface regeneration

| | |
|---|---|
| Regeneration buffer | 10 mM Glycine-HCl |
| Contact time(s) | 30 |
| Flow rate(μl/min) | 30 |

### Results:

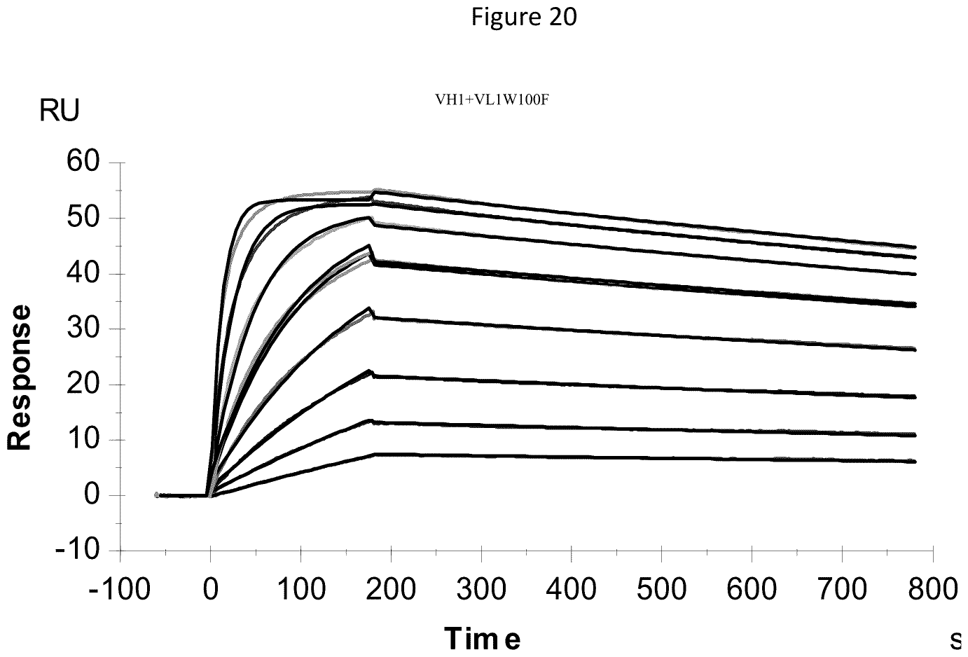

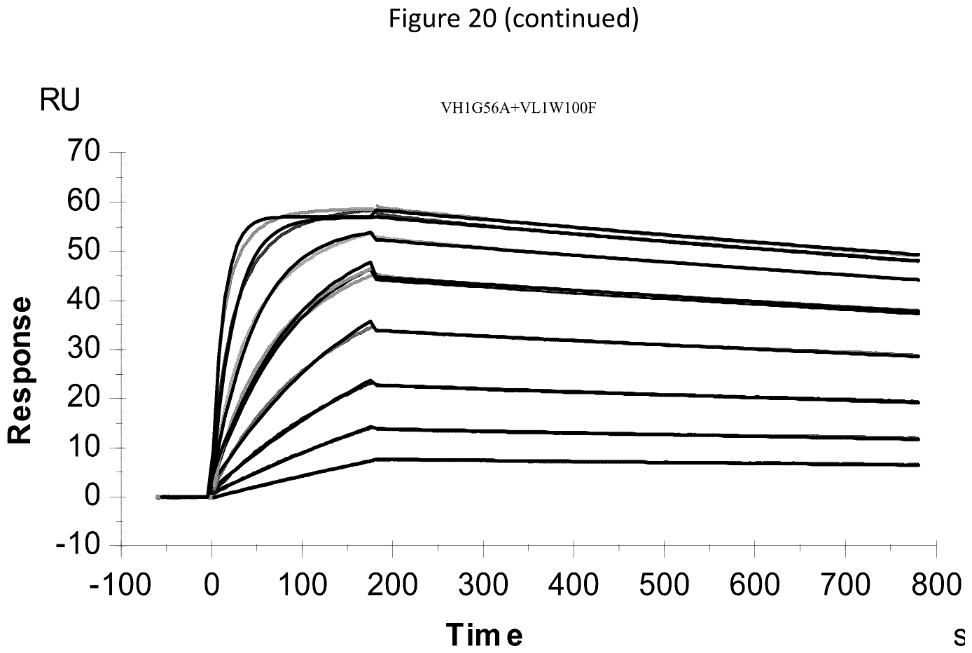

All the data were processed using the Biacore T200 Evaluation software version 3.1. Flow cell 1 and blank injection of buffer in each cycle were used as a double reference for response units subtraction. The binding kinetic data is shown in Table 16, and the binding sensor-grams are shown in FIG. 20.

TABLE 16
Affinity ranking of antibodies to Human IL13Ra2

| | | | | | Rmax | |
|---|---|---|---|---|---|---|
| Ligand | Analyte | ka (1/Ms) | kd (1/s) | KD (M) | (RU) | Chi²(RU²) |
| Vh1G56A-VL1W100F | Human IL13Ra2 | 1.02E+06 | 2.84E−04 | 2.77E−10 | 58.66 | 0.207 |
| Vh1-VL1W100F | Human IL13Ra2 | 1.03E+06 | 3.34E−04 | 3.26E−10 | 55.02 | 0.169 |

### Conclusions:

The affinity of the single and double mutant humanized antibodies have higher affinities than the parental humanized chimeric antibody and showed similar affinity to the humanized VH1-VL 1 antibodies.

### Example 6: Production of Antibody

In the following Example, the inventor assesses the binding affinity and production levels of several forms of a humanized anti-IL13Rα2 antibody described herein.

Antibodies were produced using the Expi293™ expression system (ThermoFisher) according to the manufacturer's protocol. Briefly, Expri293F TM cells were grown in Expri293F TM Expression medium in shaker flasks. Expri293F TM cells were seeded for transfection at 3×10⁶ viable cells/ml. Plasmid DNA encoding the humanized antibodies was diluted in Opti-MEM™ I medium to achieve a final concentration of 1 g/ml in cultured cells. ExpiFectamine™ 293 reagent was diluted in Opti-MEM™ I medium. In 5 minutes, diluted DNA was combined with diluted ExpiFectamine™ 293 reagent to form plasmid DNA/ExpiFectamine™ 293 complexes for 15 minutes. The complexes were then gently transferred to the cells, swirling the culture flask. The transfected cells were incubated in a 37° C. incubator with 8% CO2 and over 80% relative humidity on an orbital shaker. At 18-22 hours post-transfection, ExpiFectamine™ 293 transfection enhancer 1 and ExpiFectamine™ 293 transfection enhancer 2 were added to the transfection flask. The transfected cells were grown for 4-5 days until cells' viability decreased to approximately 50%. Cells were then collected and centrifuged at 400 g for 10 min. The supernatants containing antibodies were collected and filtered through 0.45 m filters. All antibodies were purified using protein A-sepharose 4B TM (Invitrogen).

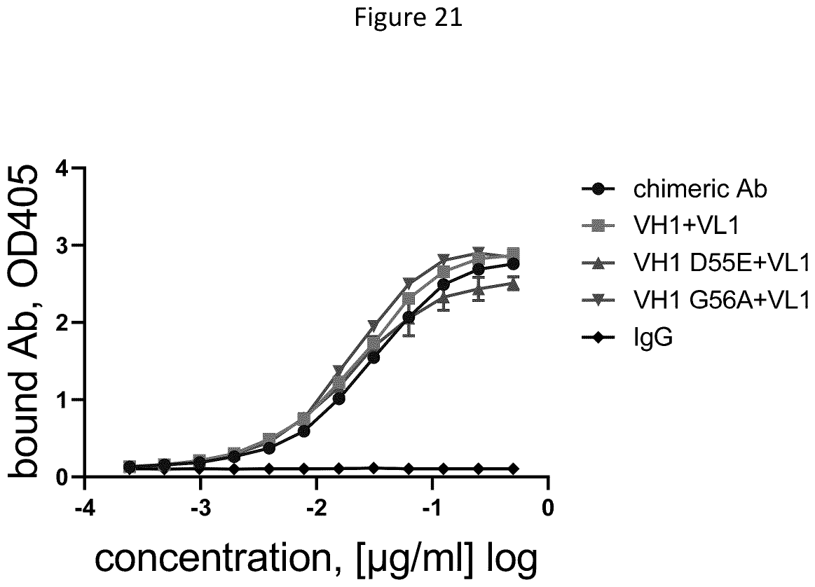

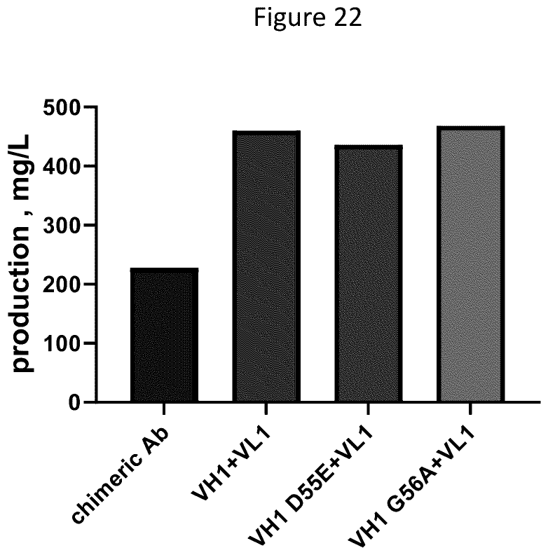

The binding affinity of the chimeric murine antibody (Ab), humanized antibody (VH1-VL 1) and variants of humanized antibody with mutations in the VH1 chain (D55E and G56A) to human recombinant IL13Rα2 was testing by plate ELISA. The results demonstrate that, while the D55E mutation affect the affinity of the antibody, the G56A mutation does not. Further, the results show that both the mutants and the non-mutant humanized antibody bind to IL13Rα2 with a greater affinity than the parental chimeric murine antibody (FIG. 21). Additionally, the production of these antibodies in a single batch was compared. The results demonstrate that all three humanized antibodies (i.e., the D55E mutant, the G56A mutant, and the non-mutated antibody) were produced at higher concentrations than the parental chimeric murine antibody (FIG. 22).

The affinity of additional humanized antibody variants with mutations in the VH1 chain (M34A, D52E) or VL 1 chain (M37A, Q58E, Q94E, W100F) was measured. While several of these variants (i.e., VH1 M34A-VL 1, VH1 MD52E-VL 1, VH1-VL 1 Q94E, and VH1-VL 1 W100F) showed comparable affinity to the non-mutated humanized antibody (VH1-VL 1), two of the tested mutilations (i.e., VH1-VL 1 M37A and VH1-VL 1 Q58E) drastically decreased binding affinity (FIG. 23) demonstrating that some changes within the humanized antibody drastically reduced the binding affinity of the humanized antibodies.

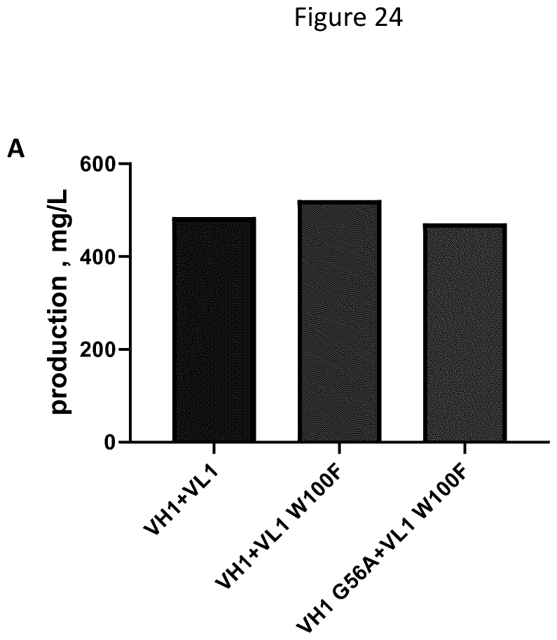

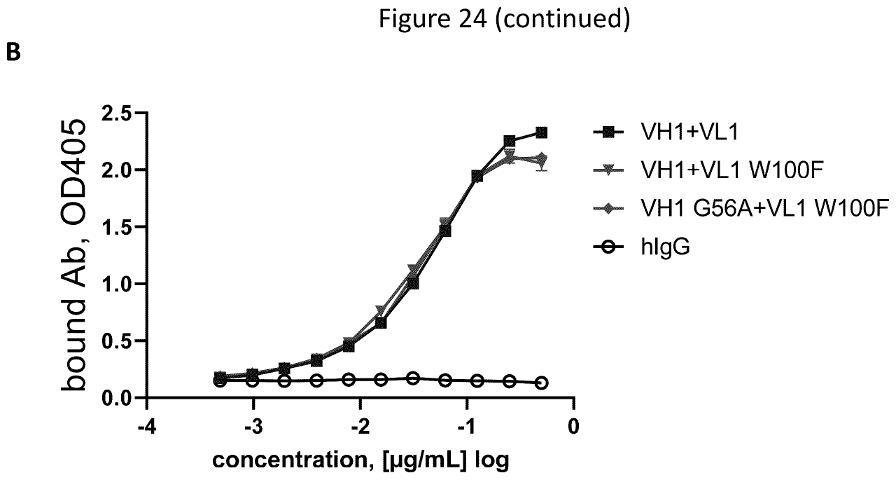

The production and binding affinity of the double mutant VH1 G56A-VL 1 W100F was also tested. This double mutant was produced at comparable levels and bound to human recombinant IL13Rα2 with a similar affinity as both the single mutant VH1-VL 1 W100F and the non-mutated humanized antibody (VH1-VL 1) (FIG. 24). These mutations found in this double mutant each offer potential therapeutic advantages, as the G56A mutation disrupts an isomerization site and the W100F mutation removes an oxidation site. Thus, antibodies and BTEs produced with these mutations are predicted to be more stable in vivo and to provide stability for storage and processing that provide benefits for large scale production, storage and distribution in providing a more stable and less aggregation-prone antibody or BTE product but still maintaining a highly pure and sufficient binder to the target IL13Ra molecule.

### Example 7: Humanized Bispecific T-Cell Engager Specific for IL13Ra (BTE) Antibody

In the following Example, the inventor describes the generation of and assessment of a Bi-specific T-cell engager (BTE) that simultaneously targets the T cell protein CD3F and the glioblastoma (GBM) protein interleukin 13 receptor alpha (L13Ra). This BTE is a bispecific fusion protein that comprises two single-chain variable fragments (scFvs) connected by flexible linker.

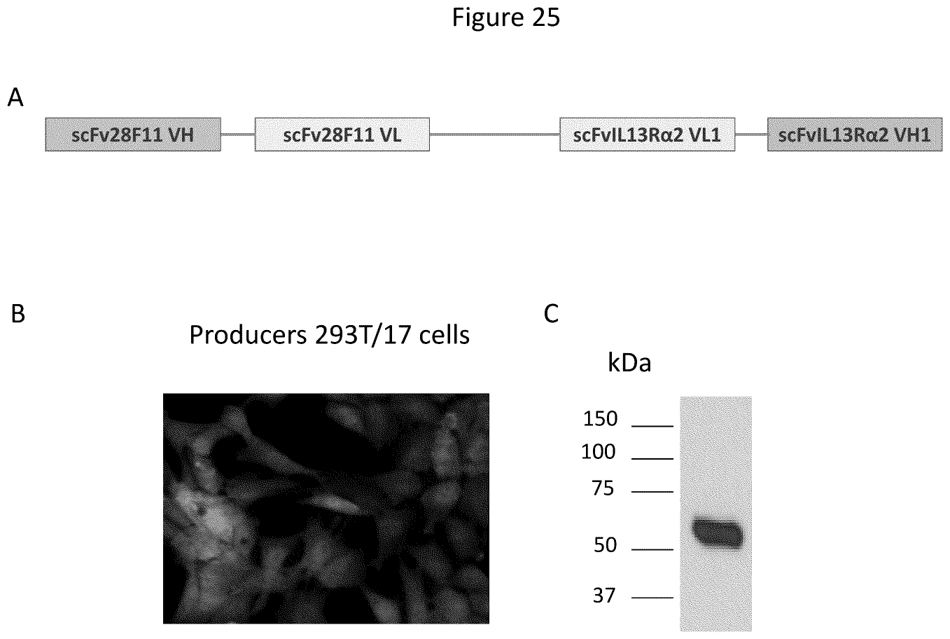

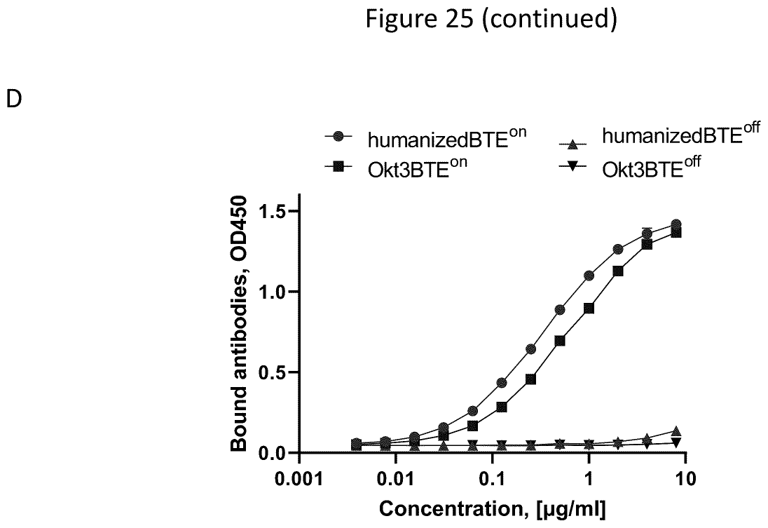

The first scFv is derived from the fully human anti-CD3 antibody 28F1 (amino acid sequences SEQ ID NO:51 and 52 and polynucleotide sequences SEQ ID NO:61 and 62 derived from the sequences described in U.S. Pat. No. 7,728,114 and Schaller et al. Pharmacokinetic Analysis of a Novel Human EGFRvIII:CD3 Bispecific Antibody in Plasma and Whole Blood Using a High-Resolution Targeted Mass Spectrometry Approach. J Proteome Res. 2019 Aug. 2; 18(8):3032-3041. doi: 10.1021/acs.jproteome.9b00145. Epub 2019 Jul 19. PMID: 31267741; PMCID: PMC7325320, FIG. 1, the contents of which are incorporated by reference in their entireties). The second scFv is derived from the humanized anti-IL13Rα2 antibody described herein, including the point mutations that increased the stability of the anti-L13Ra antibody (see schematic in FIG. 25A and particularly including in some embodiments, the point mutations G56A and W100F). Specifically, the orientation of the VH and VL domains and linker between two scFvs is important for the design of the BTE. The suitable BTEs designed have the following orientation: a-CD3 VH -linker-αCD3 VL-linker-humanized VH scFvIL13Rα2- linker humanized scFvIL13Rα2 VL. Suitable polypeptides comprising the BTEs are found in SEQ ID NO:48, SEQ ID NO:49; SEQ ID NO:56-59.

This novel humanized CD3:IL13Rα2 bi-specific antibody specifically binds IL13Rα2, but not IL13Ral, activates T cells as judged by the markers of T cells activation and specifically kill IL13Rα2 expressing GBM6 cells as demonstrated in this example.

The BTEs were generated and expressed in 293T/17 cells (FIG. 25B-C). Bispecific T cell engager (BTE) targeting IL13Rα2 was generated using single-chain variable region (scFv) described herein and scFv of the mAb 28F11 directed towards CD 3. ScFvs were connected using a flexible glycine/serine linker in the following orientation displayed in FIG. 25. BTEOFF and BTE_{ON} control molecules were generated by replacing the complementary determinant region 3 of the mAb47 light chain and heavy chain with the sequence of the mAb MOPC-21, which prevents IL13Rα2 binding. Polyhistidine (6His) tag was added at the C-terminus of BTE constructs for BTE purification and detection. An additional BTE construct using Okt3BTE was used as a control (comprising the chimeric mouse/human BTE using the mouse VH and VL in the BTE.

Lentiviral vectors (pLVX-IRES-ZsGreenl) encoding cDNA for each BTE were constructed, and the corresponding lentiviral particles were used to transduce HEK293T cells for the production of BTE proteins. Recombinant BTE proteins were purified from culture supernatants using HisPure resin. Purified BTE integrity was verified by western blotting using anti-His antibodies (FIG. 25).

BTE cDNAs were codon-optimized for human cells' expression, synthesized, and cloned in pAmp vector by the Thermo Fisher Scientific (Waltham, MA). Alexafluo647 labeling kit was used to label generated BTEs for the binding studies. Using online ExPASy tools (www.expasy.org/tools/), amino acid length and molecular weight was calculated ((“Quest Calculate™ IgG Concentration Calculator.” AAT Bioquest, Inc, 17 Jan. 2020, www.aatbio.com/tools/calculate-IgG-concentration.).

The pLVX-IRES-ZsGreenl plasmid (Takara Bio USA, Inc., Mountain View, CA) was used to generate construct encoding BTE cDNA. Briefly, the plasmid was cut with EcoRI and BamHI restriction enzymes (NEB, Ipswich, MA), and BTE cDNA excised from the pAmp vector was directly ligated with T4DNA ligase. DH5u cells (NEB, Ipswich, MA) were transformed and grown overnight on ampicillin LB agar plates. Selected colonies were grown in LB broth in the presence of 100 μg/mL of ampicillin. Plasmid DNA was purified using reagents and QIAGEN Plasmid Midi Kits protocol (Germantown, MD). Purified DNA was subjected to Sangers sequencing (GENEWIZ, South Plainfield, NJ). Lentivirus plasmids encoding for BTEs cDNA and 4th generation Lenti-X™ Packaging Single Shots (Takara Bio USA, Inc., Mountain View, CA) were used to produce lentiviral particles in HEK 293/17 cells. Cell supernatants were collected at 24 and 48 h after transfection of HEK 293/17 cells and concentrated using a LentiX concentrator (Takara Bio USA, Inc., Mountain View, CA). HEK 293/17 cells or NSC cells were plated in 6 well plates at a density of 10⁵ per well, and next day transduced with viral concentrate in the presence of 4 and 1 μg/mL polybrene, respectively (Sigma, St.Louis, MO), and cultured overnight at 37° C./5% CO₂. The following day media was replaced, and the transduced cells were expanded. Transduced 293T or NSCs were subjected to fluorescence-activated cell sorting (Robert H. Lurie Comprehensive Cancer Center Flow Cytometry Core Facility (RHLCCC), Chicago, IL) to select for cells expressing a comparable level of ZsGreenl protein among different BTE lines. Sorted cells were expanded in culture as specified. Before the collection of the supernatant containing the BTE proteins, 293T cells at 80% confluence were shifted to 32° C. and incubated for 3 days in the presence of the proteases inhibitors (Sigma, St. Louis, MO) to induced maximal protein production. NSC secreting BTE proteins were cultured at 37° C./5% CO₂.

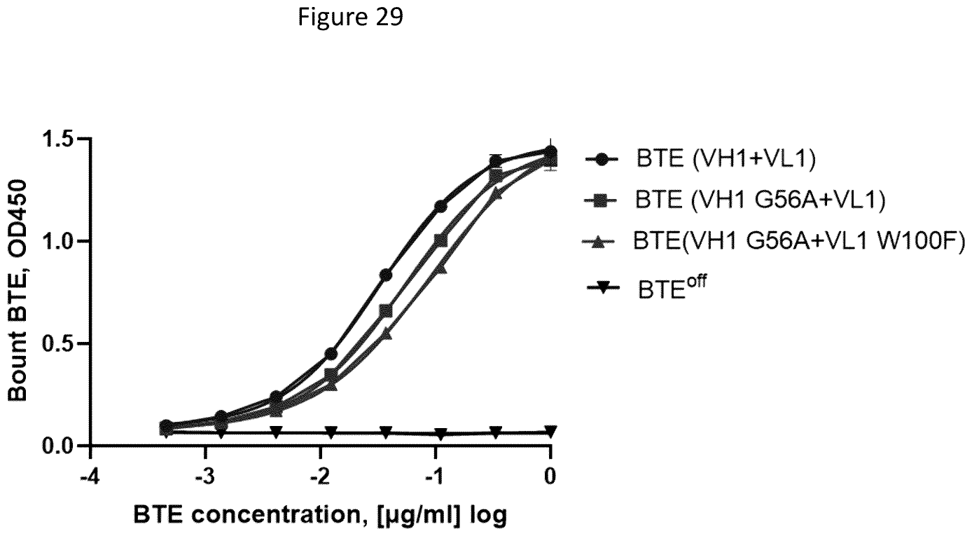

The binding affinity of the BTE to recombinant human IL13Rα2 was measured by plate ELISA and demonstrated better binding characteristics that the chimeric BTE using the mouse monoclonal Ab VH and VL domains. Enzyme-linked immunosorbent (ELISA) assay was performed to determine the binding of BTE proteins to human IL13Rα2. ELISA was performed in 96-well plates coated with 1 g/mL of human recombinant IL13Rα2hFc (cat #7147-IR-100, R&D Systems, Minneapolis, MN). Following blocking with PBS/2% FBS and washes with TBS-Tween 20 buffer (Boston Bioproducts, Ashland, MA), purified BTE proteins or NSCs supernatants were incubated for 1 h room temperature (RT) at various concentrations or dilutions. Bound BTE proteins were detected with HRP-conjugated anti-6×HIS tag antibodies (ab1187, Abcam, Cambridge, MA) using 1-Step™ Slow TMB-ELISA (Thermo Scientific, Rockford, IL) and 2N HCl according to manufacturer's directions. Plates were read at 450 nm and 570 nm using a BioTek plate reader (BioTek, Winooski, VT), and data were plotted using the Microsoft Excel program prior to analysis. ELISA was also performed for the VH1G56-VL 1 and VH1G56A-VL 1W100F as shown in FIG. 29 showing the BTE with single and double mutations bind IL12Rα2. Additional experiments show they are fully functional.

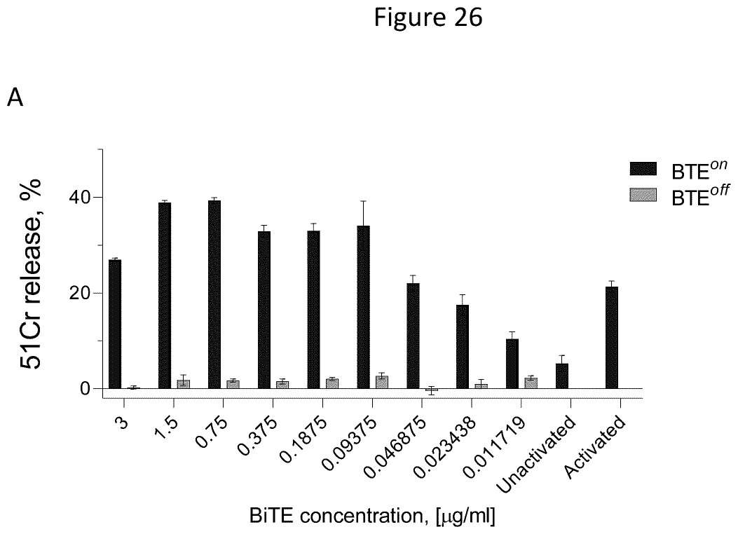

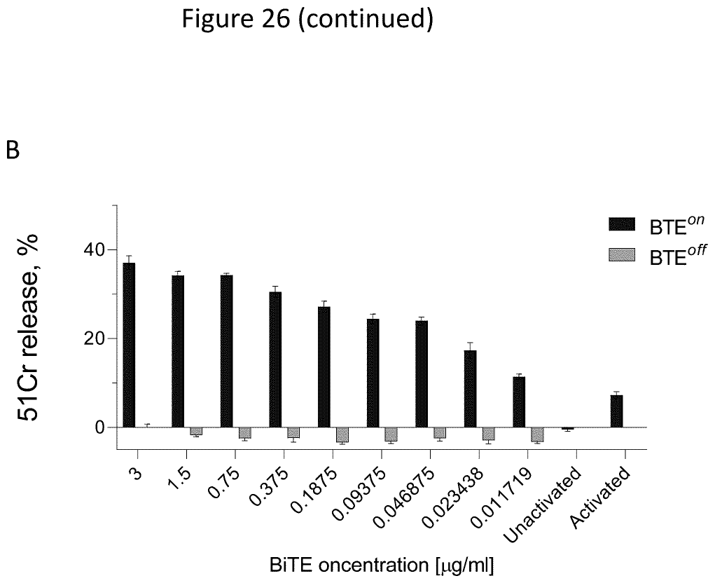

Next, a Chromium-51 (⁵¹Cr) release assay was performed to assess the ability of the BTE to assess its cytotoxicity against glioblastoma (GBM) cells. The results of this assay demonstrate that the BTE activated donor T cells (peripheral blood mononuclear cells) and killed IL13Rα2-expressing GBM6 cells (FIG. 26).

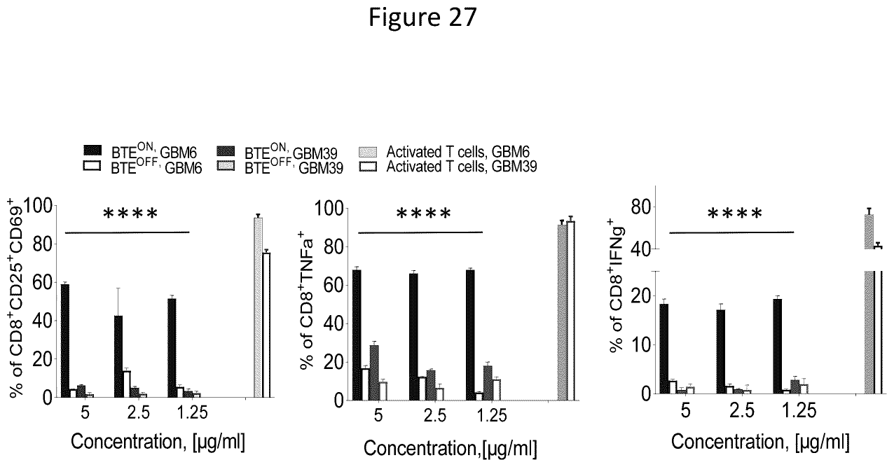

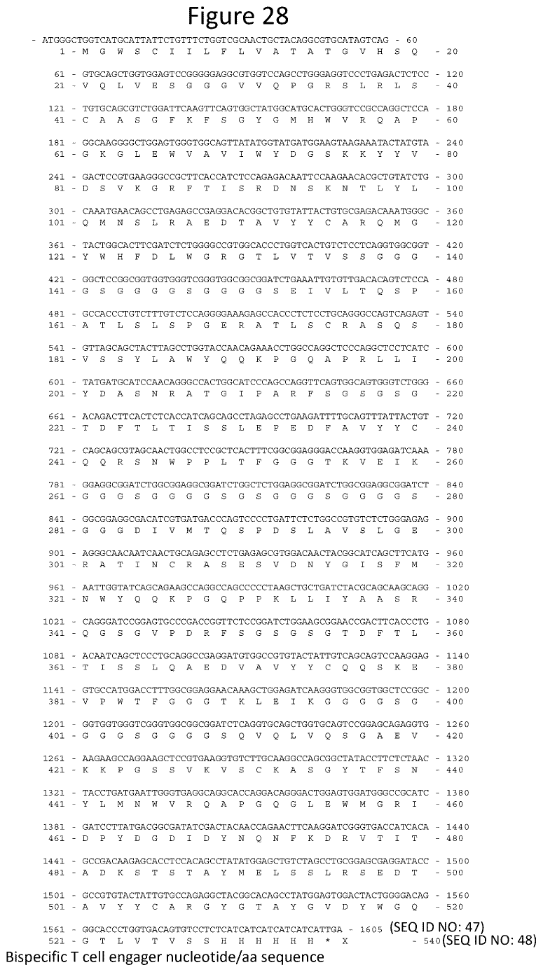

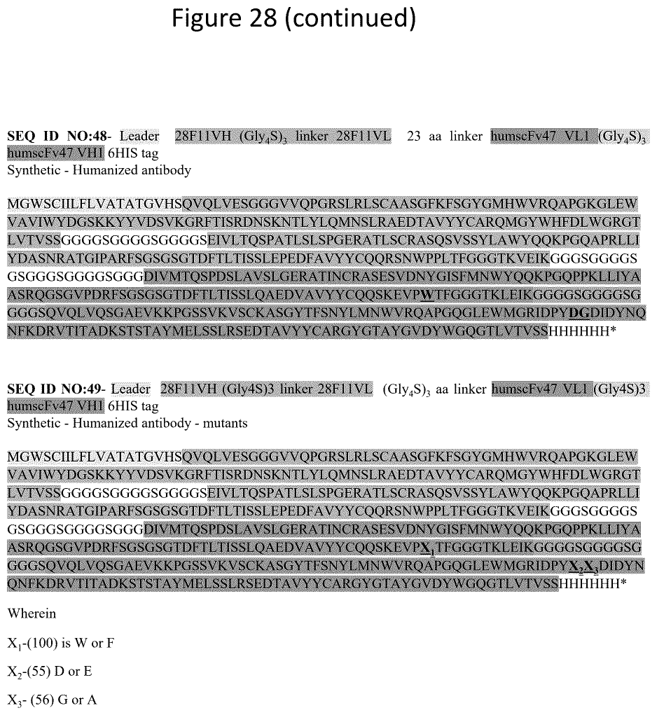

Finally, the ability of the BTE to activate T cells was tested. The results demonstrate that the BTE activates donor CD8+ T cells in co-culture with the IL13Rα2-expressing GBM6 patient-derived xenograft line, but not with IL13Rα2-negative GBM39 patient-derived xenograft line (FIG. 27). PB CD3+ and CD8+ TILs were used for the co-culture in functional assays. Two types of controls were used in all experiments: activated cells co-cultured in the presence of CD3/CD28/CD2 activating bead (STEMCELL Technologies, Vancouver, Canada) and non-activated cells co-cultured in the presence of complete media without beads. IL13Rα2+ cells of hrIL13Rα2 were used for antigenic stimulation except when noted. GBM39, IL13Rα2-negative cells or GBM-free co-cultures were used as antigen specificity controls. The co-culture experiments were at the range of the target to effector cells (T: E) ratio 1:1-1:30 as specified, where the target is glioma cells, and effectors are T cells. In experiments with BTEs, dose-response concentrations of the recombinant protein were as specified.

The cytotoxic activity of T cells against glioma cells in the presence of the BTE proteins or NSCs secreting BTE proteins was determined using a standard ⁵¹Cr release assay. Released Cr51 readings were obtained at 18-24 h of the co-culture, as previously described.

What is claimed:

1. A humanized antibody that binds to IL13Rα2 comprising:
a variable light domain (V_{L}) comprising an amino acid sequence of SEQ ID NO:53 or an amino acid with at least 95% sequence similarity to SEQ ID NO:53; and
a variable heavy domain (V_{H}) comprising an amino acid sequence of SEQ ID NO:54 or an amino acid with at least 95% sequence similarity to SEQ ID NO:54.

2. The humanized antibody of claim 1, wherein (a) X₁ in V_{L} is F; (b) X₂ in V_{H} is E, (c) X₃ in V_{H} is A; or (d) combinations of (a), (b) and (c).

3. The humanized antibody of claim 1 or 2, wherein X₁ in V_{L} is F; and X₃ in V_{H} is A.

4. The humanized antibody of any one of the preceding claims, wherein the humanized antibody is a single-chain antibody and further comprising a flexible linker between the V_{H} and V_{L} domain.

5. The humanized antibody of claim 4, wherein the linker is an amino acid sequence comprising glycine and serine from about 4-25 amino acids in length.

6. The humanized antibody of claim 4 or 5, wherein the linker comprises (G₄S)₃.

7. The humanized antibody that binds IL13Rα2 of claim 1 or 2, comprising:
(a) a variable heavy domain (V_{H}) comprising SEQ ID NO: 1, SEQ ID NO:2, SEQ ID NO:3, SEQ ID NO:4, SEQ ID NO:9, SEQ ID NO:10, SEQ ID NO: 11, SEQ ID NO: 12, or an amino acid sequence having at least 95% sequence similarity to SEQ ID NO: 1-4 or SEQ ID NO:9-12, and
(b) a variable light domain (V_{L}) comprising SEQ ID NO:5, SEQ ID NO:6, SEQ ID NO:7, SEQ ID NO:8, SEQ ID NO:13, SEQ ID NO:14, SEQ ID NO: 15, SEQ ID NO: 16, or an amino acid sequence having at least 95% sequence similarity to SEQ ID NO:5-8 or SEQ ID NO:13-16.

8. The humanized antibody of claim 7, wherein the antibody comprises:
(i) a V_{H} selected from the group consisting of SEQ ID NO:9, SEQ ID NO: 10, SEQ ID NO:11, and SEQ ID NO: 12, wherein the V_{H} has one or more mutations selected from M34L, M34A, M34I, M34V, D52E, P53A, D55E, and G56A; and
(ii) a V_{L} comprising the amino acid sequence selected from the group consisting of SEQ ID NO:13, SEQ ID NO:14, SEQ ID NO: 15, and SEQ ID NO:16, wherein the V_{L} comprising one or more mutations selected from M37L, M37I, M37V, Q58R, Q58A, Q94E, Q94R, Q94A, W100F, and W100Y.

9. The humanized antibody of any one of claims 7-8, wherein the antibody cannot isomerize and comprises:
(i) a V_{H} selected from SEQ LD NO:9, SEQ ID NO:10, SEQ ID NO:11, or SEQ ID NO:12 wherein the V_{H} comprises G56A, and a V_{L} selected from SEQ ID NO:5, SEQ ID NO:6, SEQ ID NO:7, SEQ ID NO:8, SEQ ID NO:13, SEQ ID NO:14, SEQ ID NO: 15, or SEQ ID NO:16, or
(ii) a V_{H} selected from SEQ ID NO:9, SEQ ID NO:10, SEQ ID NO: 11, or SEQ ID NO:12 wherein the V_{H} comprises D55E, and a V_{L} selected from SEQ ID NO:5, SEQ ID NO:6, SEQ ID NO:7, SEQ ID NO:8, SEQ ID NO:13, SEQ ID NO:14, SEQ ID NO: 15, or SEQ ID NO: 16.

10. The humanized antibody of claim 7, wherein the antibody comprises:
(i) SEQ ID NO:1 and SEQ ID NO: 5;
(ii) SEQ ID NO:1 and SEQ ID NO: 6;
(iii) SEQ ID NO:1 and SEQ ID NO:7;
(iv) SEQ ID NO:1 and SEQ ID NO: 8;
(v) SEQ ID NO:2 and SEQ ID NO:5;
(vi) SEQ ID NO:2 and SEQ ID NO:6;
(vii) SEQ ID NO:2 and SEQ ID NO:7;
(viii) SEQ ID NO:2 and SEQ ID NO:8;
(ix) SEQ ID NO:3 and SEQ ID NO:5
(x) SEQ ID NO:3 and SEQ ID NO:6;
(xi) SEQ ID NO:3 and SEQ ID NO:7;
(xii) SEQ ID NO:3 and SEQ ID NO:8;
(xiii) SEQ ID NO:4 and SEQ ID NO:5;
(xiv) SEQ ID NO:4 and SEQ ID NO:6;
(xv) SEQ ID NO 4 and SEQ ID NO:7; or
(xvi) SEQ ID NO. 4 and SEQ ID NO:8.

11. The humanized antibody of any one of claims 7-10, wherein the binding constant (K_{D}) of the antibody is 5×10⁻⁹ M or less.

12. The humanized antibody of any one of claims 7-11, wherein the K_{D} of the antibody is 2×10⁻¹⁰ M or less.

13. The humanized antibody of any one of claims 1-12, further comprising an agent or conjugate.

14. The humanized antibody of claim 13, wherein the agent is a therapeutic agent.

15. The humanized antibody of claim 13, wherein the agent is a detection agent.

16. The humanized antibody of any one of the preceding claims, wherein the variable heavy domain (V_{H}) and the variable light domain (V_{L}) are linked by a flexible linker.

17. The humanized antibody of any one of claims 7-16, wherein the antibody is a single-chain variable fragment antibody.

18. The humanized antibody of any one of the preceding claims, wherein the antibody comprises a signal sequence 5′ to the variable heavy domain (V_{H}).

19. The humanized antibody of claim 18, wherein the signal sequence is SEQ ID NO:29.

20. A composition comprising the humanized antibody of any one of the preceding claims and a pharmaceutically acceptable carrier.

21. A nucleic acid sequence encoding the humanized antibody of any one of claims 1-20.

22. A vector comprising the nucleic acid sequence of claim 15.

23. A method of treating an IL13Rα2-expressing cancer in a subject, the method comprising: administering a therapeutically effective amount of the humanized antibody of any one of claims 1-19 or the composition of claim 20 to treat the cancer.

24. The method of claim 23, further comprising determining whether IL13Rα2 is expressed on a cancer cell in the subject, wherein the humanized antibody or composition is only administered to the subject if IL13Rα2 expression is detected.

25. The method of claim 23 or 24, wherein the cancer is glioblastoma.

26. An engineered bispecific antibody comprising a first single-chain variable fragment (scFv) that binds to CD3 and a second scFv that binds to IL13Rα2,
wherein the first scFv comprises:
(a) a variable light domain (V_{H}) comprising an amino acid sequence of SEQ ID NO:51 or an amino acid sequence with at least 95% sequence similarity to SEQ ID NO:51; a
(b) a first flexible linker; and
(c) a variable heavy domain (V_{L}) comprising an amino acid sequence of SEQ ID NO:52 or an amino acid sequence with at least 95% sequence similarity to SEQ ID NO:52, and wherein the second scFv comprises:
(d) a variable light domain (V_{L}) comprising an amino acid sequence of SEQ ID NO:53 or an amino acid with at least 95% sequence similarity to SEQ ID NO:53;
(e) a second linker; and
(f) a variable heavy domain (V_{H}) comprising an amino acid sequence of SEQ ID NO:54 or an amino acid with at least 95% sequence similarity to SEQ ID NO:54; and
wherein the bispecific antibody comprises from 5′ to 3′: the V_{H} of the first scFv, the V_{L} of the first scFv, the V_{L} of the second scFv, and the V_{H} of the second scFv.

27. The engineered bispecific antibody of claim 26, wherein the first single-chain variable fragment (scFv) that binds to CD3 and second scFv that binds to IL13Rα2 are linked via a third flexible linker.

28. The engineered bispecific antibody of claim 26 or 27, wherein the first and second linker are an amino acid sequence of about 10-20 amino acids, the amino acids selected from glycine and serine.

29. The engineered bispecific antibody of claim 28, wherein the first and second linker are (Gly₄S)₃ (SEQ ID NO:55).

30. The engineered bispecific antibody of any one of claims 26-29, wherein the third linker is a 20-30 amino acid glycine-serine linker.

31. The engineered bispecific antibody of claim 30, wherein the third linker is SEQ ID NO:56.

32. The engineered bispecific antibody of any one of claims 26-31, wherein amino acid sequence further comprises a signal peptide on the 5′ end, preferably wherein the signal peptide is SEQ ID NO: 50.

33. The engineered bispecific antibody of any one of claims 26-32, wherein the wherein the V_{H} of the second scFv comprises a mutation, wherein the mutation is X₂ is E, X₃ is A, or a combination thereof.

34. The engineered bispecific antibody of claim 33, wherein the V_{H} of the second scFv comprises a mutation, wherein the mutation is X₃ is A, or a combination thereof

35. The engineered bispecific antibody of any one of claims 26-34, further comprising a tag.

36. The engineered bispecific antibody of any one of claims 26-34, wherein the engineered bispecific T cell engager comprises an amino acid of SEQ ID NO: 49.

37. The engineered bispecific antibody of claim 36, wherein the engineered bispecific T cell engager comprises:
an amino acid sequence selected from the group consisting of SEQ ID NO:49, SEQ ID NO: 57, SEQ ID NO: 58, SEQ ID NO: 59, SEQ ID NO:60.

38. The engineered bispecific antibody of claim 36, wherein the amino acid sequence is SEQ ID NO: 57.

39. The engineered bispecific antibody of claim 36, wherein the amino acid sequence is SEQ ID NO:58.

40. A composition comprising the bispecific antibody of any one of claims 26-39 and a pharmaceutically acceptable carrier.

41. A nucleic acid sequence encoding the bispecific antibody of any one of claims 26-39.

42. A vector comprising the nucleic acid sequence of claim 41.

43. A transgenic neural stem cell (NSC) that expresses the bispecific antibody of any one of claims 26-39.

44. A method of treating an IL13Rα2-expressing cancer in a subject, the method comprising: administering a therapeutically effective amount of the bispecific antibody of any one of claims 26-39, the composition of claim 40, or the transgenic NSCs of claim 43 to the subject to treat the cancer.

45. The method of claim 44 further comprising determining whether IL13Rα2 is expressed on a cancer cell in the subject, wherein the humanized antibody or composition is only administered to the subject if IL13Rα2 expression is detected.

46. The method of claim 44 or 45, wherein the cancer is a glioblastoma.

47. A method for inducing lysis of a target cell comprising contacting the target cell with a bispecific antibody of any one of claims 26-39 in the presence of a T cell in an amount effective to lyse the target cell.

48. The method of claim 47, wherein the T cell is a cytotoxic T cell.

49. The method of claim 47 or 48, wherein the method further comprises administering the bispecific antibody in vivo to a subject having cancer.

50. The humanized antibody of claim 13, wherein the humanized antibody forms an antibody-conjugate.

51. The humanized antibody of claim 13, wherein the humanized antibody and agent are genetically fused as a fusion protein.


SEQUENCE LISTING (62 sequences)

SEQ ID NO: 1 - protein, 449 aa, synthetic construct
  REGION 1..449: note = Synthetic - pCDNA3.4-IgG1-VH1
QVQLVQSGAEVKKPGSSVKVSCKASGYTFSNYLMNWVRQAPGQGLEWMGRIDPYDGDIDYNQNFKDRVTITADKSTSTAYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 449 aa, synthetic construct
  REGION 1..449: note = Synthetic - pCDNA3.4-IgG1-VH2
QVQLVQSGAEVKKPGASVKVSCKASGYTFSNYLMNWVRQAPGQRLEWMGRIDPYDGDIDYNQNFKDRVTITRDTSASTAYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 449 aa, synthetic construct
  REGION 1..449: note = Synthetic - pCDNA3.4-IgG1-VH3
QVQLVQSGAEVKKPGASVKVSCKASGYTFSNYLMNWVRQAPGQGLEWMGRIDPYDGDIDYNQNFKDRVTMTRDTSTSTVYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 449 aa, synthetic construct
  REGION 1..449: note = Synthetic - pCDNA3.4-IgG1-VH4
QVQLVQSGAEVKKPGASVKVSCKASGYTFSNYLMNWVRQATGQGLEWMGRIDPYDGDIDYNQNFKDRVTITRNTSISTAYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 218 aa, synthetic construct
  REGION 1..218: note = Synthetic - pCDNA3.4-IgG1-VL1
DIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 218 aa, synthetic construct
  REGION 1..218: note = Synthetic - pCDNA3.4-IgG1-VL2
DIQMTQSPSSLSASVGDRVTITCRASESVDNYGISFMNWYQQKPGKAPKLLIYAASRQGSGVPSRFSGSGSGTDFTLTISSLQPEDFATYYCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 218 aa, synthetic construct
  REGION 1..218: note = Synthetic - pCDNA3.4-IgG1-VL3
EIVLTQSPATLSLSPGERATLSCRASESVDNYGISFMNWYQQKPGQAPRLLIYAASRQGSGIPARFSGSGSGTDFTLTISSLEPEDFAVYYCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 218 aa, synthetic construct
  REGION 1..218: note = Synthetic - pCDNA3.4-IgG1-VL4
EIVMTQSPATLSVSPGERATLSCRASESVDNYGISFMNWYQQKPGQAPRLLIYAASRQGSGIPARFSGSGSGTEFTLTISSLQSEDFAVYYCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 449 aa, synthetic construct
  REGION 1..449: note = Synthetic - Modified pCDNA3.4-IgG1-VH1
  VARIANT 34: note = X is M, L, A, I, or V
  VARIANT 52: note = X is D or E
  VARIANT 53: note = X is P or A
  VARIANT 55: note = X is D or E
  VARIANT 56: note = X is G or A
QVQLVQSGAEVKKPGSSVKVSCKASGYTFSNYLXNWVRQAPGQGLEWMGRIXXYXXDIDYNQNFKDRVTITADKSTSTAYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 449 aa, synthetic construct
  REGION 1..449: note = Synthetic - Modified pCDNA3.4-IgG1-VH2
  VARIANT 34: note = X is M, L, A, I, or V
  VARIANT 52: note = X is D or E
  VARIANT 53: note = X is P or A
  VARIANT 55: note = X is D or E
  VARIANT 56: note = X is G or A
QVQLVQSGAEVKKPGASVKVSCKASGYTFSNYLXNWVRQAPGQRLEWMGRIXXYXXDIDYNQNFKDRVTITRDTSASTAYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 449 aa, synthetic construct
  REGION 1..449: note = Synthetic - Modified pCDNA3.4-IgG1-VH3
  VARIANT 34: note = X is M, L, A, I, or V
  VARIANT 52: note = X is D or E
  VARIANT 53: note = X is P or A
  VARIANT 55: note = X is D or E
  VARIANT 56: note = X is G or A
QVQLVQSGAEVKKPGASVKVSCKASGYTFSNYLXNWVRQAPGQGLEWMGRIXXYXXDIDYNQNFKDRVTMTRDTSTSTVYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 449 aa, synthetic construct
  REGION 1..449: note = Synthetic - Modified pCDNA3.4-IgG1-VH4
  VARIANT 34: note = X is M, L, A, I, or V
  VARIANT 52: note = X is D or E
  VARIANT 53: note = X is P or A
  VARIANT 55: note = X is D or E
  VARIANT 56: note = X is G or A
QVQLVQSGAEVKKPGASVKVSCKASGYTFSNYLXNWVRQATGQGLEWMGRIXXYXXDIDYNQNFKDRVTITRNTSISTAYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 218 aa, synthetic construct
  REGION 1..218: note = Synthetic - Modified pCDNA3.4-IgG1-VL1
  VARIANT 37: note = X is M, L, A, I, or V
  VARIANT 58: note = X is Q, E, R, or A
  VARIANT 94: note = X is Q, E, R, or A
  VARIANT 100: note = X is W, F, or Y
DIVMTQSPDSLAVSLGERATINCRASESVDNYGISFXNWYQQKPGQPPKLLIYAASRXGSGVPDRFSGSGSGTDFTLTISSLQAEDVAVYYCQXSKEVPXTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 218 aa, synthetic construct
  REGION 1..218: note = Synthetic - Modified pCDNA3.4-IgG1-VL2
  VARIANT 37: note = X is M, L, A, I, or V
  VARIANT 58: note = X is Q, E, R, or A
  VARIANT 94: note = X is Q, E, R, or A
  VARIANT 100: note = X is W, F, or Y
DIQMTQSPSSLSASVGDRVTITCRASESVDNYGISFXNWYQQKPGKAPKLLIYAASRXGSGVPSRFSGSGSGTDFTLTISSLQPEDFATYYCQXSKEVPXTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 218 aa, synthetic construct
  REGION 1..218: note = Synthetic - Modified pCDNA3.4-IgG1-VL3
  VARIANT 37: note = X is M, L, A, I, or V
  VARIANT 58: note = X is Q, E, R, or A
  VARIANT 94: note = X is Q, E, R, or A
  VARIANT 100: note = X is W, F, or Y
EIVLTQSPATLSLSPGERATLSCRASESVDNYGISFXNWYQQKPGQAPRLLIYAASRXGSGIPARFSGSGSGTDFTLTISSLEPEDFAVYYCQXSKEVPXTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 218 aa, synthetic construct
  REGION 1..218: note = Synthetic - Modified pCDNA3.4-IgG1-VL4
  VARIANT 37: note = X is M, L, A, I, or V
  VARIANT 58: note = X is Q, E, R, or A
  VARIANT 94: note = X is Q, E, R, or A
  VARIANT 100: note = X is W, F, or Y
EIVMTQSPATLSVSPGERATLSCRASESVDNYGISFXNWYQQKPGQAPRLLIYAASRXGSGIPARFSGSGSGTEFTLTISSLQSEDFAVYYCQXSKEVPXTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 1350 bp, synthetic construct
  misc_feature 1..1350: note = Synthetic - pCDNA3.4-IgG1-VH1
  source 1..1350: mol_type = other DNA
caggtgcagctggtgcagtccggagcagaggtgaagaagccaggaagctccgtgaaggtgtcttgcaaggccagcggctataccttctctaactacctgatgaattgggtgaggcaggcaccaggacagggactggagtggatgggccgcatcgatccttatgacggcgatatcgactacaaccagaacttcaaggatcgggtgaccatcacagccgacaagagcacctccacagcctatatggagctgtctagcctgcggagcgaggataccgccgtgtactattgtgccagaggctacggcacagcctatggagtggactactggggacagggcaccctggtgacagtgtcctctgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtga

SEQ ID NO: 18 - DNA, 1350 bp, synthetic construct
  misc_feature 1..1350: note = Synthetic - pCDNA3.4-IgG1-VH2
  source 1..1350: mol_type = other DNA
caggtgcagctggtgcagagcggagcagaggtgaagaagccaggagccagcgtgaaggtgtcctgcaaggcctctggctataccttctccaactacctgatgaattgggtgaggcaggcaccaggacagcgcctggagtggatgggccggatcgatccttatgacggcgatatcgactacaaccagaacttcaaggatcgggtgaccatcacaagagacacctccgcctctacagcctatatggagctgagctccctgaggtctgaggataccgccgtgtactattgtgcaaggggatacggaacagcatatggagtggactactggggacagggcaccctggtgacagtgtctagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtga

SEQ ID NO: 19 - DNA, 1350 bp, synthetic construct
  misc_feature 1..1350: note = Synthetic - pCDNA3.4-IgG1-VH3
  source 1..1350: mol_type = other DNA
caggtgcagctggtgcagagcggagcagaggtgaagaagccaggagccagcgtgaaggtgtcctgcaaggcctctggctatacattctccaactacctgatgaattgggtgcggcaggcaccaggacagggactggagtggatgggcaggatcgatccttatgacggcgatatcgactacaaccagaacttcaaggatcgggtgaccatgacaagagacacctccacatctaccgtgtatatggagctgagctccctgaggtctgaggatacagccgtgtactattgtgcaaggggatacggaaccgcatatggagtggactactggggccagggcacactggtgaccgtgtctagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtga

SEQ ID NO: 20 - DNA, 1350 bp, synthetic construct
  misc_feature 1..1350: note = Synthetic - pCDNA3.4-IgG1-VH4
  source 1..1350: mol_type = other DNA
caggtgcagctggtgcagagcggagcagaggtgaagaagccaggagccagcgtgaaggtgtcctgcaaggcctctggctataccttctccaactacctgatgaattgggtgcggcaggcaacaggacagggactggagtggatgggcaggatcgatccttatgacggcgatatcgactacaaccagaacttcaaggaccgggtgaccatcacaagaaacacctccatctctacagcctatatggagctgagctccctgaggtctgaggataccgccgtgtactattgtgcaaggggatacggaacagcatatggagtggactactggggacagggcaccctggtgacagtgtctagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtga

SEQ ID NO: 21 - DNA, 657 bp, synthetic construct
  misc_feature 1..657: note = Synthetic - pCDNA3.4-IgG1-VL1
  source 1..657: mol_type = other DNA
gacatcgtgatgacccagtcccctgattctctggccgtgtctctgggagagagggcaacaatcaactgcagagcctctgagagcgtggacaactacggcatcagcttcatgaattggtatcagcagaagccaggccagccccctaagctgctgatctacgcagcaagcaggcagggatccggagtgcccgaccggttctccggatctggaagcggaaccgacttcaccctgacaatcagctccctgcaggccgaggatgtggccgtgtactattgtcagcagtccaaggaggtgccatggacctttggcggaggaacaaagctggagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctga

SEQ ID NO: 22 - DNA, 657 bp, synthetic construct
  misc_feature 1..657: note = Synthetic - pCDNA3.4-IgG1-VL2
  source 1..657: mol_type = other DNA
gacatccagatgacccagtctcctagctccctgtccgcctctgtgggcgacagggtgaccatcacatgcagagccagcgagtccgtggataactacggcatctccttcatgaattggtatcagcagaagcccggcaaggcccctaagctgctgatctacgcagcatctaggcagggaagcggagtgccctcccggttctctggcagcggctccggcaccgactttaccctgacaatctctagcctgcagccagaggatttcgccacatactattgtcagcagagcaaggaggtgccatggacctttggcggaggaacaaagctggagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctga

SEQ ID NO: 23 - DNA, 657 bp, synthetic construct
  misc_feature 1..657: note = Synthetic - pCDNA3.4-IgG1-VL3
  source 1..657: mol_type = other DNA
gagatcgtgctgacccagtccccagccacactgtctctgagcccaggagagagggccaccctgtcttgcagagcctccgagtctgtggacaactacggcatcagcttcatgaattggtatcagcagaagccaggacaggcacctaggctgctgatctacgcagcaagcaggcagggatccggaatccccgcccggttcagcggctccggctctggcacagactttaccctgacaatcagctccctggagcctgaggatttcgccgtgtactattgtcagcagtctaaggaggtgccatggacctttggcggcggcacaaagctggagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctga

SEQ ID NO: 24 - DNA, 657 bp, synthetic construct
  misc_feature 1..657: note = Synthetic - pCDNA3.4-IgG1-VL4
  source 1..657: mol_type = other DNA
gagatcgtgatgacccagtccccagccacactgtctgtgagcccaggagagagggccaccctgtcctgcagagcctccgagtctgtggacaactacggcatctctttcatgaattggtatcagcagaagccaggacaggcacctaggctgctgatctacgcagcaagcaggcagggatccggaatccctgcacggttcagcggatccggatctggcacagagtttaccctgacaatcagctccctgcagtctgaggatttcgccgtgtactattgtcagcagagcaaggaggtgccatggacctttggcggaggaacaaagctggagatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctga

SEQ ID NO: 25 - protein, 119 aa, synthetic construct
  REGION 1..119: note = Synthetic - VH region from the original mouse monoclonal antibody (mAb)
QVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSS

SEQ ID NO: 26 - protein, 111 aa, synthetic construct
  REGION 1..111: note = Synthetic - VL region from the original mouse monoclonal antibody (mAb)
DIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIK

SEQ ID NO: 27 - protein, 449 aa, synthetic construct
  REGION 1..449: note = Synthetic - pCDNA3.4-IgG1-VH1 with D55E mutation
QVQLVQSGAEVKKPGSSVKVSCKASGYTFSNYLMNWVRQAPGQGLEWMGRIDPYDGEIDYNQNFKDRVTITADKSTSTAYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 449 aa, synthetic construct
  REGION 1..449: note = Synthetic - pCDNA3.4-IgG1-VH1 with G56A mutation
QVQLVQSGAEVKKPGSSVKVSCKASGYTFSNYLMNWVRQAPGQGLEWMGRIDPYDADIDYNQNFKDRVTITADKSTSTAYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 19 aa, synthetic construct
  REGION 1..19: note = Synthetic - Signal sequence from mouse Ig heavy chain V region 102
MGWSCIILFLVATATGVHS

SEQ ID NO: 30 - protein, 468 aa, synthetic construct
  REGION 1..468: note = Synthetic - pCDNA3.4-IgG1-VH1 with signal sequence
MGWSCIILFLVATATGVHSQVQLVQSGAEVKKPGSSVKVSCKASGYTFSNYLMNWVRQAPGQGLEWMGRIDPYDGDIDYNQNFKDRVTITADKSTSTAYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 468 aa, synthetic construct
  REGION 1..468: note = Synthetic - pCDNA3.4-IgG1-VH2 with signal sequence
MGWSCIILFLVATATGVHSQVQLVQSGAEVKKPGASVKVSCKASGYTFSNYLMNWVRQAPGQRLEWMGRIDPYDGDIDYNQNFKDRVTITRDTSASTAYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 468 aa, synthetic construct
  REGION 1..468: note = Synthetic - pCDNA3.4-IgG1-VH3 with signal sequence
MGWSCIILFLVATATGVHSQVQLVQSGAEVKKPGASVKVSCKASGYTFSNYLMNWVRQAPGQGLEWMGRIDPYDGDIDYNQNFKDRVTMTRDTSTSTVYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 468 aa, synthetic construct
  REGION 1..468: note = Synthetic - pCDNA3.4-IgG1-VH4 with signal sequence
MGWSCIILFLVATATGVHSQVQLVQSGAEVKKPGASVKVSCKASGYTFSNYLMNWVRQATGQGLEWMGRIDPYDGDIDYNQNFKDRVTITRNTSISTAYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 237 aa, synthetic construct
  REGION 1..237: note = Synthetic - pCDNA3.4-IgG1-VL1 with signal sequence
MGWSCIILFLVATATGVHSDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 237 aa, synthetic construct
  REGION 1..237: note = Synthetic - pCDNA3.4-IgG1-VL2 with signal sequence
MGWSCIILFLVATATGVHSDIQMTQSPSSLSASVGDRVTITCRASESVDNYGISFMNWYQQKPGKAPKLLIYAASRQGSGVPSRFSGSGSGTDFTLTISSLQPEDFATYYCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 237 aa, synthetic construct
  REGION 1..237: note = Synthetic - pCDNA3.4-IgG1-VL3 with signal sequence
MGWSCIILFLVATATGVHSEIVLTQSPATLSLSPGERATLSCRASESVDNYGISFMNWYQQKPGQAPRLLIYAASRQGSGIPARFSGSGSGTDFTLTISSLEPEDFAVYYCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 237 aa, synthetic construct
  REGION 1..237: note = Synthetic - pCDNA3.4-IgG1-VL4 with signal sequence
MGWSCIILFLVATATGVHSEIVMTQSPATLSVSPGERATLSCRASESVDNYGISFMNWYQQKPGQAPRLLIYAASRQGSGIPARFSGSGSGTEFTLTISSLQSEDFAVYYCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 468 aa, synthetic construct
  REGION 1..468: note = Synthetic - Generic pCDNA3.4-IgG1-VH1 with signal sequence
  VARIANT 53: note = X is M, L, A, I, or V
  VARIANT 71: note = X is D or E
  VARIANT 72: note = X is P or A
  VARIANT 74: note = X is D or E
  VARIANT 75: note = X is G or A
MGWSCIILFLVATATGVHSQVQLVQSGAEVKKPGSSVKVSCKASGYTFSNYLXNWVRQAPGQGLEWMGRIXXYXXDIDYNQNFKDRVTITADKSTSTAYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 468 aa, synthetic construct
  REGION 1..468: note = Synthetic - Generic pCDNA3.4-IgG1-VH2 with signal sequence
  VARIANT 53: note = X is M, L, A, I, or V
  VARIANT 71: note = X is D or E
  VARIANT 72: note = X is P or A
  VARIANT 74: note = X is D or E
  VARIANT 75: note = X is G or A
MGWSCIILFLVATATGVHSQVQLVQSGAEVKKPGASVKVSCKASGYTFSNYLXNWVRQAPGQRLEWMGRIXXYXXDIDYNQNFKDRVTITRDTSASTAYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 468 aa, synthetic construct
  REGION 1..468: note = Synthetic - Generic pCDNA3.4-IgG1-VH3 with signal sequence
  VARIANT 53: note = X is M, L, A, I, or V
  VARIANT 71: note = X is D or E
  VARIANT 72: note = X is P or A
  VARIANT 74: note = X is D or E
  VARIANT 75: note = X is G or A
MGWSCIILFLVATATGVHSQVQLVQSGAEVKKPGASVKVSCKASGYTFSNYLXNWVRQAPGQGLEWMGRIXXYXXDIDYNQNFKDRVTMTRDTSTSTVYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 468 aa, synthetic construct
  REGION 1..468: note = Synthetic - Generic pCDNA3.4-IgG1-VH4 with signal sequence
  VARIANT 53: note = X is M, L, A, I, or V
  VARIANT 71: note = X is D or E
  VARIANT 72: note = X is P or A
  VARIANT 74: note = X is D or E
  VARIANT 75: note = X is G or A
MGWSCIILFLVATATGVHSQVQLVQSGAEVKKPGASVKVSCKASGYTFSNYLXNWVRQATGQGLEWMGRIXXYXXDIDYNQNFKDRVTITRNTSISTAYMELSSLRSEDTAVYYCARGYGTAYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 237 aa, synthetic construct
  REGION 1..237: note = Synthetic - Generic pCDNA3.4-IgG1-VL1 with signal sequence
  VARIANT 56: note = X is M, L, A, I, or V
  VARIANT 77: note = X is Q, E, R, or A
  VARIANT 113: note = X is Q, E, R, or A
  VARIANT 119: note = X is W, F, or Y
MGWSCIILFLVATATGVHSDIVMTQSPDSLAVSLGERATINCRASESVDNYGISFXNWYQQKPGQPPKLLIYAASRXGSGVPDRFSGSGSGTDFTLTISSLQAEDVAVYYCQXSKEVPXTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 237 aa, synthetic construct
  REGION 1..237: note = Synthetic - Generic pCDNA3.4-IgG1-VL2 with signal sequence
  VARIANT 56: note = X is M, L, A, I, or V
  VARIANT 77: note = X is Q, E, R, or A
  VARIANT 113: note = X is Q, E, R, or A
  VARIANT 119: note = X is W, F, or Y
MGWSCIILFLVATATGVHSDIQMTQSPSSLSASVGDRVTITCRASESVDNYGISFXNWYQQKPGKAPKLLIYAASRXGSGVPSRFSGSGSGTDFTLTISSLQPEDFATYYCQXSKEVPXTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 237 aa, synthetic construct
  REGION 1..237: note = Synthetic - Generic pCDNA3.4-IgG1-VL3 with signal sequence
  VARIANT 56: note = X is M, L, A, I, or V
  VARIANT 77: note = X is Q, E, R, or A
  VARIANT 113: note = X is Q, E, R, or A
  VARIANT 119: note = X is W, F, or Y
MGWSCIILFLVATATGVHSEIVLTQSPATLSLSPGERATLSCRASESVDNYGISFXNWYQQKPGQAPRLLIYAASRXGSGIPARFSGSGSGTDFTLTISSLEPEDFAVYYCQXSKEVPXTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 237 aa, synthetic construct
  REGION 1..237: note = Synthetic - Generic pCDNA3.4-IgG1-VL4 with signal sequence
  VARIANT 56: note = Xis M, L, A, I, or V
  VARIANT 77: note = X is Q, E, R, or A
  VARIANT 113: note = X is Q, E, R, or A
  VARIANT 119: note = X is W, F, or Y
MGWSCIILFLVATATGVHSEIVMTQSPATLSVSPGERATLSCRASESVDNYGISFXNWYQQKPGQAPRLLIYAASRXGSGIPARFSGSGSGTEFTLTISSLQSEDFAVYYCQXSKEVPXTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - DNA, 57 bp, synthetic construct
  misc_feature 1..57: note = Synthetic - Nucleotide sequence of signal sequence
  source 1..57: mol_type = other DNA
atgggctggtcatgcattattctgtttctggtcgcaactgctacaggcgtgcatagt

SEQ ID NO: 47 - DNA, 1605 bp, synthetic construct
  misc_feature 1..1605: note = Synthetic - Humanized BTE (scFv28F11-23 aa linker-scFv47)
  source 1..1605: mol_type = other DNA
atgggctggtcatgcattattctgtttctggtcgcaactgctacaggcgtgcatagtcaggtgcagctggtggagtccgggggaggcgtggtccagcctgggaggtccctgagactctcctgtgcagcgtctggattcaagttcagtggctatggcatgcactgggtccgccaggctccaggcaaggggctggagtgggtggcagttatatggtatgatggaagtaagaaatactatgtagactccgtgaagggccgcttcaccatctccagagacaattccaagaacacgctgtatctgcaaatgaacagcctgagagccgaggacacggctgtgtattactgtgcgagacaaatgggctactggcacttcgatctctggggccgtggcaccctggtcactgtctcctcaggtggcggtggctccggcggtggtgggtcgggtggcggcggatctgaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgctcactttcggcggagggaccaaggtggagatcaaaggaggcggatctggcggaggcggatctggctctggaggcggatctggcggaggcggatctggcggaggcgacatcgtgatgacccagtcccctgattctctggccgtgtctctgggagagagggcaacaatcaactgcagagcctctgagagcgtggacaactacggcatcagcttcatgaattggtatcagcagaagccaggccagccccctaagctgctgatctacgcagcaagcaggcagggatccggagtgcccgaccggttctccggatctggaagcggaaccgacttcaccctgacaatcagctccctgcaggccgaggatgtggccgtgtactattgtcagcagtccaaggaggtgccatggacctttggcggaggaacaaagctggagatcaagggtggcggtggctccggcggtggtgggtcgggtggcggcggatctcaggtgcagctggtgcagtccggagcagaggtgaagaagccaggaagctccgtgaaggtgtcttgcaaggccagcggctataccttctctaactacctgatgaattgggtgaggcaggcaccaggacagggactggagtggatgggccgcatcgatccttatgacggcgatatcgactacaaccagaacttcaaggatcgggtgaccatcacagccgacaagagcacctccacagcctatatggagctgtctagcctgcggagcgaggataccgccgtgtactattgtgccagaggctacggcacagcctatggagtggactactggggacagggcaccctggtgacagtgtcctctcatcatcatcatcatcattga

SEQ ID NO: 48 - protein, 534 aa, synthetic construct
  REGION 1..534: note = Synthetic - Humanized BTE (scFv28F11-23 aa linker-scFv47)
MGWSCIILFLVATATGVHSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGSGGGGSGSGGGSGGGGSGGGDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPWTFGGGTKLEIKGGGGSGGGGSGGGGSQVQLVQSGAEVKKPGSSVKVSCKASGYTFSNYLMNWVRQAPGQGLEWMGRIDPYDGDIDYNQNFKDRVTITADKSTSTAYMELSSLRSEDTAVYYCARGYGTAYGVDYWGQGTLVTVSSHHHHHH

SEQ ID NO: 49 - protein, 534 aa, synthetic construct
  REGION 1..534: note = Synthetic - Generic humanized BTE
  VARIANT 383: note = X is W or F, preferably F
  VARIANT 464: note = X is D or E
  VARIANT 465: note = X G or A, preferably A
MGWSCIILFLVATATGVHSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGSGGGGSGSGGGSGGGGSGGGDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPXTFGGGTKLEIKGGGGSGGGGSGGGGSQVQLVQSGAEVKKPGSSVKVSCKASGYTFSNYLMNWVRQAPGQGLEWMGRIDPYXXDIDYNQNFKDRVTITADKSTSTAYMELSSLRSEDTAVYYCARGYGTAYGVDYWGQGTLVTVSSHHHHHH

SEQ ID NO: 50 - protein, 19 aa, synthetic construct
  REGION 1..19: note = Synthetic - Leader peptide used in BTE
MGWSCIILFLVATATGVHS

SEQ ID NO: 51 - protein, 116 aa, synthetic construct
  REGION 1..116: note = Synthetic - Heavy chain of 28F11 used in BTE
QVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTV

SEQ ID NO: 52 - protein, 108 aa, synthetic construct [sequence omitted]

SEQ ID NO: 53 - protein, 111 aa, synthetic construct
  REGION 1..111: note = Synthetic - Humanized light chain of scFvIL13Ra2 used in BTE
  VARIANT 100: note = X is W or F
DIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPXTFGGGTKLEIK

SEQ ID NO: 54 - protein, 119 aa, synthetic construct
  REGION 1..119: note = Synthetic - Humanized heavy chain of scFvIL13Ra2 used in BTE
  VARIANT 55: note = X is D or E
  VARIANT 56: note = X is G or A
QVQLVQSGAEVKKPGSSVKVSCKASGYTFSNYLMNWVRQAPGQGLEWMGRIDPYXXDIDYNQNFKDRVTITADKSTSTAYMELSSLRSEDTAVYYCARGYGTAYGVDYWGQGTLVTVSS

SEQ ID NO: 55 - protein, 15 aa, synthetic construct
  REGION 1..15: note = Synthetic - Gly4 S linker used in BTE (linker 1 or 3)
GGGGSGGGGSGGGGS

SEQ ID NO: 56 - protein, 23 aa, synthetic construct
  REGION 1..23: note = Synthetic - Linker 2 used in BTE
GGGSGGGGSGSGGGSGGGGSGGG

SEQ ID NO: 57 - protein, 534 aa, synthetic construct
  REGION 1..534: note = Synthetic - Humanized BTE with G56A mutation
MGWSCIILFLVATATGVHSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGSGGGGSGSGGGSGGGGSGGGDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPWTFGGGTKLEIKGGGGSGGGGSGGGGSQVQLVQSGAEVKKPGSSVKVSCKASGYTFSNYLMNWVRQAPGQGLEWMGRIDPYDADIDYNQNFKDRVTITADKSTSTAYMELSSLRSEDTAVYYCARGYGTAYGVDYWGQGTLVTVSSHHHHHH

SEQ ID NO: 58 - protein, 534 aa, synthetic construct
  REGION 1..534: note = Synthetic - Humanized BTE with W100F and G56A mutations
MGWSCIILFLVATATGVHSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGSGGGGSGSGGGSGGGGSGGGDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPFTFGGGTKLEIKGGGGSGGGGSGGGGSQVQLVQSGAEVKKPGSSVKVSCKASGYTFSNYLMNWVRQAPGQGLEWMGRIDPYDADIDYNQNFKDRVTITADKSTSTAYMELSSLRSEDTAVYYCARGYGTAYGVDYWGQGTLVTVSSHHHHHH

SEQ ID NO: 59 - protein, 534 aa, synthetic construct
  REGION 1..534: note = Synthetic - Humanized BTE with W100F and D55E mutations
MGWSCIILFLVATATGVHSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGSGGGGSGSGGGSGGGGSGGGDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPFTFGGGTKLEIKGGGGSGGGGSGGGGSQVQLVQSGAEVKKPGSSVKVSCKASGYTFSNYLMNWVRQAPGQGLEWMGRIDPYEGDIDYNQNFKDRVTITADKSTSTAYMELSSLRSEDTAVYYCARGYGTAYGVDYWGQGTLVTVSSHHHHHH

SEQ ID NO: 60 - protein, 534 aa, synthetic construct
  REGION 1..534: note = Synthetic - Humanized BTE with W100F, D55E, and G56A mutations
MGWSCIILFLVATATGVHSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGSGGGGSGSGGGSGGGGSGGGDIVMTQSPDSLAVSLGERATINCRASESVDNYGISFMNWYQQKPGQPPKLLIYAASRQGSGVPDRFSGSGSGTDFTLTISSLQAEDVAVYYCQQSKEVPFTFGGGTKLEIKGGGGSGGGGSGGGGSQVQLVQSGAEVKKPGSSVKVSCKASGYTFSNYLMNWVRQAPGQGLEWMGRIDPYEADIDYNQNFKDRVTITADKSTSTAYMELSSLRSEDTAVYYCARGYGTAYGVDYWGQGTLVTVSSHHHHHH

SEQ ID NO: 61 - DNA, 354 bp, synthetic construct
  misc_feature 1..354: note = Synthetic - 28F11 VH nucleotide sequence
  source 1..354: mol_type = other DNA
caggtgcagctggtggagtccgggggaggcgtggtccagcctgggaggtccctgagactctcctgtgcagcgtctggattcaagttcagtggctatggcatgcactgggtccgccaggctccaggcaaggggctggagtgggtggcagttatatggtatgatggaagtaagaaatactatgtagactccgtgaagggccgcttcaccatctccagagacaattccaagaacacgctgtatctgcaaatgaacagcctgagagccgaggacacggctgtgtattactgtgcgagacaaatgggctactggcacttcgatctctggggccgtggcaccctggtcactgtctcctca

SEQ ID NO: 62 - DNA, 324 bp, synthetic construct
  misc_feature 1..324: note = Synthetic - 28F11 VL nucleotide sequence
  source 1..324: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgctcactttcggcggagggaccaaggtggagatcaaa